(19) United States
(12) Patent Application Publication
(10) Pub. No.: US 2004/0260068 A1
(43) Pub. Date: Dec. 23, 2004

(54) Humanized chicken antibodies

(76) Inventors: Naoya Tsurushita, Palo Alto, CA (US); Shankar Kumar, Pleasanton, CA (US); Maximiliano Vasquez, Palo Alto, CA (US)

Correspondence Address: HOWREY SIMON ARNOLD & WHITE, LLP, C/O M.P. DROSOS, DIRECTOR OF IP ADMINISTRATION, 2941 FAIRVIEW PK, BOX 7, FALLS CHURCH, VA, 22042, US

(21) Appl. No.: 10/788,625

(22) Filed: Feb. 26, 2004

Related U.S. Application Data
(60) Provisional application No. 60/451,622, filed on Feb. 28, 2003.

(51) Int. Cl. C07K 16/44

(52) U.S. Cl. 530/388.15

(57) ABSTRACT

The present invention concerns chicken antibodies and humanized chicken antibodies and the method of making such antibodies. In a particular embodiment, it provides humanized chicken antibodies that bind to or neutralize human and/or mouse proteins, such as IL-12 or L-selectin. It also provides the method for the prevention or treatment of autoimmune diseases by using such antibodies.

### CROSS-REFERENCES TO RELATED APPLICATIONS

This application claims priority as a non-provisional application of U.S. Provisional Application Ser. No. 60/451,622, filed Feb. 28, 2003.

### FIELD OF THE INVENTION

The present invention relates to the field of immunology and protein chemistry. In particular, it concerns chicken antibodies and “humanized” chicken antibodies that bind to or neutralize human or/and mouse proteins, such as IL-12 or L-selectin, and the prevention or treatment of cancer or autoimmune diseases by using such antibodies.

### BACKGROUND OF THE INVENTION

The development of monoclonal antibodies (mAbs) has brought great progress in biological research and clinical science (Kohler, G. et al., Nature 256: 495-497 (1975)). Monoclonal antibodies are used for the diagnosis, prevention, or treatment of various diseases such as cancer, autoimmune disease, viral infection, and prevention of tissue rejection in organ transplant. Human monoclonal antibodies are the most desirable as long-term therapeutic agents. However, the source of human antibodies is not always available and often severely limited.

To overcome such a problem, murine (mouse) monoclonal antibodies (mAbs) are widely used as substitutes for human antibodies. Murine mAbs are generally produced by hybridoma technology. However, such a method is not satisfactory in raising mAb against mouse proteins. It is also difficult to generate the mAb against a large number of human proteins that are highly conserved in mammalian evolution and, therefore render a limited immune response in mice due to immunological tolerance invoked during fetal development. The chicken therefore becomes a very desirable immunological host, since it is located on a different branch from mammals on the phylogenetic tree and is known to have immunologic potency comparable to that of mammals (Gassmann, M. et al., FASEB J. 4: 2528 (1990); Larsson, A. et al., Comp. Immunol. Microbiol. Infect. Dis.13:199 (1990); Carroll, S. B. et al., J. Biol. Chem. 258:24 (1983); Assoka, H. et al., Immunol.Lett.32:91 (1992); Pink, J. et al., Immunol. Rev. 91:115 (1986)).

Chicken mAbs bind strongly to a wide range of mammalian proteins including human, mouse, rabbit, etc. They are capable of binding to both human and other mammalian proteins, particularly to antigens highly conserved in mammals (Gassmann, M. et al.). Chicken IgGs have been reported to be superior in various immunological assays because they do not react with protein A, protein G, or rheumatoid factors (Katz, D. et al., J. Virol. Methods 12: 59 (1985); Larsson, A. et al., J. Immunol. Methods 113:93 (1988); Langone, J.J. et al., J. Immunol. Methods 63: 145(1983); Lasson, A. et al., J. Immunol. Methods 108: 205 (1988)).

It is also desirable in clinical development to produce an antibody that binds to a target antigen of human as well as that of other species (e.g., non-human primates, mouse, rat, rabbit, etc.), which will be used for a disease model. Such antibody can be used for both pre-clinical studies with a model animal and clinical studies with human. However, making a mouse monoclonal antibody that binds to an antigen present in human and mouse is extremely difficult because sometimes the tolerance mechanism of mouse immune system does not allow the mouse to produce antibodies against its own proteins. In contrast, it is much easier to raise chicken antibodies that bind to an antigen of multiple mammals (such as human and mouse) due to the much less conservation of antigens between the chicken and the mammal. Injection of a certain human protein into a chicken raises antibodies that recognize the protein of other species (Gassmann, et al.).

Another major problem of the clinical use of non-human antibodies is immunogenicity due to foreign species origin. This may result in a neutralizing antibody response, which is particularly problematic if therapy requires repeated administration, e.g., for treatment of a chronic or recurrent disease condition. Non-human monoclonal antibodies furthermore have a relatively short circulating half-life in humans, and often lack important human effector functions associated with the immunoglobulin constant domain.

In an effort to eliminate or reduce such problems, methods have been developed for the production of “humanized” murine antibodies that are less immunogenic but retain the antigen-binding properties of the original (i.e., non-human) antibody molecule. The retention of antigen binding properties of the non-human monoclonal antibody can potentially be achieved by grafting the nonhuman complementarity determining regions (CDRs) onto human framework regions (FRs) and constant regions, usually accompanied by substitution of critical non-human framework residues (Jones, et al., Nature 321: 522 (1986); Verhoeyen et al., Science 239: 1534 (1988); Riechmann et al., Nature 332: 323-327 (1988); Queen et al., Proc. Natl. Acad. Sci. USA 86: 10023-10029 (1989); Co and Queen, Nature 351: 501-502 (1991); U.S. Pat. Nos. 6,180,370; 5,585,089; 5,530,101, each of which is incorporated by reference in its entirety). Researchers have also attempted to produce humanized rabbit antibodies for the generation of therapeutic human antibodies (Rader, C., et al. J. Biol. Chem. 275:13668-76 (2000); Steinberg, P., et al. J. Biol. Chem. 275:36073-78 (2000)).

There are many advantages to using chickens for generating monoclonal antibodies. Chicken antibodies are known to bind strongly to a wide range of mammalian proteins, including human, mouse, rat and rabbit proteins. Therefore it is possible to use chicken antibodies that bind to highly conserved mammalian proteins, or epitopes. It is also possible for chicken antibodies to bind the antigens of multiple mammalian species, such as human and mouse. Such a monoclonal antibody could be used for animal disease models and treatment of human diseases. Such an antibody may also bind and block multiple, functionally related proteins present in an individual animal.

Humanization of mouse antibodies has been shown to greatly reduce the immunogenicity in human host. It would be also desirable to humanize chicken antibodies to enhance their human characteristics. Nevertheless, humanization of chicken antibodies has its unique technical obstacles compared to humanization of mouse antibodies.

First, chicken is more evolutionally distant from human than mouse, so that the difference between chicken and human V genes in amino acid sequence and three dimensional structure is expected to be much larger than that between mouse and human V genes.

Second, chicken V-lambda genes, compared to mouse and human V-lambda genes, carry two amino acid deletions at the N-terminus of mature proteins and one amino acid insertion in the framework 2. The presence of such deletions/insertions in the chicken V-lambda genes makes the prediction of a three dimensional structure of chicken variable regions more difficult.

The present invention has successfully generated humanized chicken antibodies and showed that humanization of chicken monoclonal antibodies is possible. The present invention also has successfully generated antibodies capable of binding to a plurality of mammalian proteins. The present invention opens a new avenue to obtain humanized antibodies against antigens conserved in mammals.

IL-12, formerly known as cytotoxic lymphocyte maturation factor, is a cytokine that stimulates proliferation of PHA-activated human peripheral blood lymphoblasts and synergizes with low concentrations of IL-2 in the induction of lymphokine-activated killer cells. IL-12 is a 75-kDa heterodimer composed of disulfide-bonded 40-kDa (p40) and 35-kDa (p35) subunits. Neutralizing antibodies of IL-12 can be used for therapeutic intervention in a number of disease states that are aggravated by activated T-cells and NK cells, such as autoimmune diseases, psoriasis, graft versus host disease and rheumatoid arthritis (U.S. Pat. Nos. 5,648,467; 5,811,523; 5,780,597; 6,300,478; and 6,410,824, each of which is incorporated by reference in its entirety). However, humanized anti-IL-12 chicken antibodies have not been developed to provide the desired human characteristics for the treatment of such disorders.

The present invention uses anti-IL-12 antibody as an example to demonstrate the humanization of chicken antibodies. A chicken anti-IL-12 monoclonal antibody was first isolated. The humanized version of this antibody is developed using the methods set forth in this invention.

### SUMMARY OF THE INVENTION

The present invention provides a humanized immunoglobulin having complementarity determining regions (CDRs) from a donor immunuglobulin, heavy chain variable region frameworks from a human acceptor immunoglobulin and light chain variable region frameworks from a human acceptor immunoglobulin, wherein said humanized immunoglobulin specifically binds to an antigen of the donor immunoglobulin, wherein said donor immunoglobulin comprises a lambda light chain, wherein said donor immunoglobulin is a chicken immunoglobulin.

Preferably, said humanized immunoglobulin specifically binds to the antigen of the donor immunoglobulin with an affinity constant of at least 10⁷ M⁻¹, 10⁸ M⁻¹, 10⁹ M⁻¹, or 10¹⁰M⁻¹.

Preferably, said humanized immunoglobulin binds to or neutralizes human IL-12 or/and mouse IL-12.

Preferably, the antigen or epitope is one found in mammals. More preferably, the antigen or epitope is found in a mammalian protein. Even more preferably, the mammalian protein is found in more than one mammalian species. Preferably, the antigen or epitope is one that is found in more than one mammalian species. Preferably, the antigen or epitope is common to more than one mammalian protein.

Preferably, the antigen is a conserved antigen found in multiple, functionally-related proteins. Preferably, the multiple, functionally-related proteins share substantial sequence similarity or homology and belong to a protein family. More preferably, the protein family is the selectin protein family, and the multiple, functionally-related proteins are E-selectin, P-selectin and L-selectin.

The present invention also provides a chicken antibody capable of blocking multiple, functionally-related proteins. Preferably, the chicken antibody is humanized.

The present invention further provides a pharmaceutical composition comprising the humanized chicken antibody and a pharmaceutical carrier, and a method of treating autoimmune disease in a subject in need of such a treatment comprising administering the pharmaceutical composition in a therapeutically effective amount.

The present invention further provides for a method of producing a humanized immunoglobulin comprising: (a) preparing expression vector(s) comprising DNA segments encoding a heavy chain variable region of the humanized immunoglobulin having complementarity determining regions (CDRs) from a donor immunoglobulin and heavy chain variable region frameworks from a human acceptor immunoglobulin, and/or DNA segments encoding a light chain variable region of the humanized immunoglobulin having complementarity determining regions (CDRs) from a donor immunoglobulin light chain variable region frameworks from a human acceptor immunoglobulin, wherein said donor immunoglobulin is a chicken immunoglobulin, (b) transforming host cells with said vector(s); and (c) culturing said transformed host cells to produce said humanized immunoglobulin.

### DESCRIPTION OF THE DRAWINGS

FIG. 1 depicts the structure of the phagemid display vector pNT3206. (A) A schematic diagram of pNT3206. Symbols used: Amp, β-lactamase gene for ampicillin resistance; pUC ori, replication origin of pUC19; lacP, E. coli lac promoter; pelB, pelB signal peptide; linker, synthetic region coding a short polypeptide linker to connect VH and Vλ; Cλ; constant region of human λ light chain gene; TAG, amber termination codon; Δcp3, carboxyl-terminal domain of M13 gene III minor coat protein. Arrows show direction of transcription. The diagram is not drawn to scale. (B) Amino acid sequence surrounding the cloning sites for VH and Vλ. Amino acid sequence is shown in single letter code. An arrow shows the cleavage site of the signal peptide. Locations of relevant restriction enzyme sites are indicated.

FIG. 2 depicts a schematic structure of mammalian expression vectors pVg1.d and pVλ2. Symbols used: CMV-P, human cytomegalovirus immediate early promoter; polyA, polyadenylation signal; SV40 polyA, SV40 polyadenylation signal; SV40-P, SV40 early promoter; pBR322 ori, replication origin of pBR322; Amp, β-lactamase gene; gpt, E. coli gpt gene; dhfr; mouse dhfr gene; Cλ2, constant region of human λ2 gene; CH1, hinge, CH2 and CH3, constant regions of human γ1 gene. Arrows show direction of transcription. Locations of relevant enzyme sites are indicated.

FIG. 3 depicts schematic structure of pHuIL12p75.rgdE for expression of human IL-12 in mammalian cells. Each of the cDNA's encoding human IL-12 p35 and p40 is placed under the regulation of the human cytomegalovirus immediate early promoter (CMV-P). The plasmid also carries the gpt gene (gpt) under the regulation of the SV40 early promoter (SV40-P) for selection in mammalian cells. Other symbols: Amp, β-lactamase gene for selection in E. coli; pBR322 ori, replication origin of pBR322; (A)_{SV40}, SV40 polyadenylation site; (A)κ, polyadenylation site of the human kappa light chain gene; (A)γ1, polyadenylation site of human gamma-1 heavy chain gene. Arrows show orientation of transcription. The figure is not drawn to scale.

FIG. 4 depicts a schematic structure of pDL220 for expression of human IL-12Rβ2-chicken Fcγ fusion proteins in mammalian cells. The cDNA encoding the extracellular region of human-IL-12 receptor β2 chain (IL-12Rβ2) was fused to the coding region of chicken Fcγ and placed under the regulation of the human cytomegalovirus immediate early promoter (CMV-P). The plasmid also carries the gpt gene (gpt) under the regulation of the SV40 early promoter (SV40-P) for selection in mammalian cells. Other symbols: Amp, β-lactamase gene for selection in E. coli; pBR322 ori, replication origin of pBR322; (A)_{SV40}, SV40 polyadenylation site; (A)γ1, polyadenylation site of human gamma-1 heavy chain gene. Arrows show orientation of transcription. Locations of relevant restriction enzyme sites are shown. The figure is not drawn to scale.

FIG. 5 depicts the alignment of the V region amino acid sequences. (A) Amino acid sequences of the Vλ, regions of chimeric B1 (SEQ ID NO:4), humanized B1 (SEQ ID NO:16), and the germline DPL16 and Jλ2 segments are shown in single letter code. (B) Amino acid sequences of the VH regions of chimeric B1 (SEQ ID NO:2), humanized B1 (SEQ ID NO:14), and the germline DP-54 and JH1 segments are shown. The CDR sequences based on the definition of Kabat are underlined in the chimeric B1 Vλ and VH sequences. The CDR sequences in the acceptor human V segments are omitted in the figure. Asterisks indicate gaps in the alignment. Note that an amino acid at position 10 is missing in both human and chicken Vλ sequences. The single underlined amino acids in the humanized Vλ and VH sequences were predicted to contact the CDR sequences and therefore substituted with the corresponding chicken residues. The double underlined amino acids in the acceptor human V segments were substituted with consensus human residues of the corresponding V subgroups to reduce potential immunogenicity. Numbers written vertically show amino acid positions according to the scheme of Kabat, Sequences of Proteins of Immunological Interest (National Institutes of Health, Bethesda, Md., 1987 and 1991). Chicken Vλ regions carry an extra amino acid at position 39A compared to human Vλ regions.

FIG. 6 depicts a scheme for conversion of Vλ and VH genes of phage antibody to mini-exons for expression in mammalian cells. The signal peptide-coding region of the Vk (or VH) of mouse anti-human CD33 monoclonal antibody M195 was amplified by PCR in such a way that the 5′ end carries an MluI site and the 3′ end is attached to a sequence homologous to the 5′ end of the B1 Vλ (or VH) coding region (fragment A). The Vλ (or VH) of chicken scFv antibody B1 was amplified by PCR in such a way that the 5′ end is attached to a sequence homologous to the 3′ end of the signal peptide-coding region of M195 Vk (or VH) and the 3′ end carries a splicing donor signal and a XbaI site (fragment B). The fragments A and B for each of B1 Vλ and VH were combined and amplified by PCR to make a mini-exon flanked by MluI and XbaI sites (fragment C).

FIG. 7 depicts the nucleotide sequence and deduced amino acid sequence of the light (A) or heavy (B) chain variable region of chicken anti-IL-12 antibody B1 in the mini exon. The nucleotide sequences shown are flanked by MluI (ACGCGT) (SEQ ID NO:87)and XbaI (TCTAGA) (SEQ ID NO:88) sites. The signal peptide sequences are in italics. The CDRs based on the definition of Kabat are underlined. The mature light and heavy chains both begin with an alanine residue (double-underlined).

FIG. 8 depicts the scheme for the synthesis of Vλ and VH mini-exons. A series of 8 overlapping oligonucleotides (1˜8) were used. Oligonucleotides 1 and 2, 3 and 4, 5 and 6, and 7 and 8 were separately annealed and extended with the Klenow fragment of DNA polymerase I. The resulting double-stranded DNA fragments, A and B, and C and D, were separately mixed, denatured, annealed and extended to yield the DNA fragments E and F, respectively, which were then mixed to generate the entire mini-exon (G) in the third annealing-and-extension step. The mini-exon was amplified by PCR with primers 9 and 10. The resulting fragments carry the flanking MluI and XbaI sites.

FIG. 9 depicts the synthetic oligonucleotides used for construction of the humanized B1 light (A) and heavy (B) chain variable region mini exons (SEQ ID Nos:27-46).

FIG. 10 depicts the nucleotide sequence and deduced amino acid sequence of the light (A) or heavy (B) chain variable region of humanized anti-IL-12 antibody B1 (HuB1) in the mini exon. The nucleotide sequences shown are flanked by MluI (ACGCGT) (SEQ ID NO:87) and XbaI (TCTAGA) (SEQ ID NO:88) sites. The signal peptide sequences, derived from the corresponding chimeric B1 mini-exons, are in italics. The CDRs based on the definition of Kabat are underlined. The mature light and heavy chains begin with double-underlined serine and glutamic acid residues, respectively. The splicing donor sequences were derived from the corresponding chimeric B1 mini-exons. The intron sequences are in italics.

FIG. 11 depicts the binding of humanized and chimeric B1 antibodies to various proteins. Binding of humanized and chimeric B1 to human IL-12, mouse IL-12, chicken lysozyme, human globin, bovine albumin, and concanavalin A was analyzed by ELISA as described in Materials and Methods of Example 3.

FIG. 12 depicts the binding of humanized and chimeric B1 antibodies to human IL-12 (A) and mouse IL-12 (B). ELISA experiments were performed as described in Materials and Methods of Example 3.

FIG. 13 depicts the comparison of the affinity to human IL-12 (A) and mouse IL-12 (B) between humanized and chimeric B1 by competition ELISA. The binding of biotinylated humanized B1 to human or mouse IL-12 was analyzed in the presence of different amounts of competitor chimeric or humanized B1 as described in Materials and Methods of Example 3.

FIG. 14 depicts the nucleotide sequence and deduced amino acid sequence of the light (A) and heavy (B) chain variable region mini exons of chicken-human chimeric anti-IL-12 antibody DD2. The nucleotide sequences shown are flanked by MluI (ACGCGT) (SEQ ID NO:87) and XbaI (TCTAGA) (SEQ ID NO:88) sites. The signal peptide sequences are in italics. The CDRs based on the definition of Kabat are underlined. The mature light and heavy chains both begin with an alanine residue (double-underlined).

FIG. 15 depicts the alignment of the V region amino acid sequences. (A) Amino acid sequences of the Vλ regions of chicken DD2 (SEQ ID NO:47), humanized DD2 (SEQ ID NO:49), and the human acceptor germline V and J segments are shown in single letter code. (B) Amino acid sequences of the VH regions of chicken DD2 (SEQ ID NO:48), humanized DD2 (SEQ ID NO:50), and the human acceptor germline V and J segments are shown in single letter code. The CDR sequence based on the definition of Kabat, et al. are underlined in the chicken DD2 Vλ and VH sequences. The CDR sequences in the acceptor human V segments are omitted in the figure. Asterisks indicate gaps in the alignment. Note that an amino acid at position 10 is missing in both human and chicken Vλ sequences. The single underlined amino acids in the humanized Vλ and VH sequences were predicted to contact the CDR and therefore substituted with the corresponding chicken residues. Numbers written vertically show amino acid positions according to the scheme of Kabat, Sequences of Proteins of Immunological Interest (National Institutes of Health, Bethesda, Md., 1987 and 1991). The location of an extra amino acid in the framework 2 of chicken Vλ is designated 39A.

FIG. 16 depicts the synthetic oligonucleotides used for construction of the humanized DD2 light (A) (Primers 1-10 are SEQ ID NOs: 51-60, respectively) and heavy (B) (Primers 1-10 are SEQ ID NOs: 61-70, respectively) chain variable region mini exons.

FIG. 17 depicts the nucleotide sequence and deduced amino acid sequence of the light (A) and heavy (B) chain variable region mini exons of humanized anti-IL-12 antibody DD2. The nucleotide sequence encoding the HuDD2 VL mini exon is SEQ ID NO:71. The amino acid sequence of the HuDD2 VL mini exon is SEQ ID NO:72. The nucleotide sequence encoding the HuDD2 VH mini exon is SEQ ID NO:73. The amino acid sequence of the HuDD2 VH mini exon is SEQ ID NO:74. The nucleotide sequences shown are flanked by MluI (ACGCGT) (SEQ ID NO:87) and XbaI (TCTAGA) (SEQ ID NO:88) sites. The signal peptide sequences are in italics. The CDRs based on the definition of Kabat are underlined. The mature light and heavy chains both begin with an alanine residue (double-underlined).

FIG. 18 depicts the binding of HuDD2 and ChDD2 to various proteins. Binding of humanized and chimeric DD2 to human IL-12, mouse IL-12, human globin, chicken lysozyme, and concanavalin A was analyzed by ELISA as described in Example 4.

FIG. 19 depicts the affinity to human IL-12 (A) and mouse IL-12 (B) between HuDD2 and ChDD2 by competition ELISA. The binding of biotinylated ChDD2 to human or mouse IL-12 was analyzed in the presence of different amounts of competitor HuDD2 or ChDD2 as described in Example 4.

FIG. 20 depicts the affinity of the variant HuDD2 antibodies to human IL-12 by competition ELISA. The binding of biotinylated ChDD2 to human IL-12 was analyzed in the presence of different amounts of competitor HuDD2 VH mutants (panel A) and VL mutants (panel B) as described in Example 4.

FIG. 21 depicts the nucleotide sequence and deduced amino acid sequence of the light (A) and heavy (B) chain variable region mini exons of chicken-human chimeric anti-L-selectin antibody D3. The nucleotide sequence encoding the D3 VL mini exon is SEQ ID NO:75. The amino acid sequence of the D3 VL mini exon is SEQ ID NO:76. The nucleotide sequence encoding the D3 VH mini exon is SEQ ID NO:77. The amino acid sequence of the D3 VH mini exon is SEQ ID NO:78. The nucleotide sequences shown are flanked by MluI (ACGCGT) (SEQ ID NO:87) and XbaI (TCTAGA) (SEQ ID NO:88) sites. The signal peptide sequences are in italics. The CDRs based on the definition of Kabat are underlined. The mature light and heavy chains both begin with an alanine residue (double-underlined).

FIG. 22 depicts the alignment of the V amino acid sequences. (A) Amino acid sequences of the Vλ regions of chicken D3 (SEQ ID NO:79), humanized D3 (SEQ ID NO:80), and the human acceptor are shown in single letter code. (B) Amino acid sequences of the VH regions of chicken D3 (SEQ ID NO:81), humanized D3 (SEQ ID NO:82), and the human acceptor are shown in single letter code. The CDR sequence based on the definition of Kabat, et al. are underlined in the chicken D3 Vλ and VH sequences. The CDR sequences in the acceptor human V segments are omitted in the figure. Asterisks indicate gaps in the alignment. Note that an amino acid at position 10 is missing in both human and chicken Vλ sequences. The single underlined amino acids in the humanized Vλ and VH sequences were predicted to contact the CDR and therefore substituted with the corresponding chicken residues. Numbers written vertically show amino acid positions according to the scheme of Kabat, Sequences of Proteins of Immunological Interest (National Institutes of Health, Bethesda, Md., 1987 and 1991). The location of an extra amino acid in the framework 2 of chicken Vλ is designated 39A.

FIG. 23 depicts the nucleotide sequence and deduced amino acid sequence of the light (A) and heavy (B) chain variable region mini exons of humanized anti-L-selectin antibody D3. The nucleotide sequence encoding the HuD3 VL mini exon is SEQ ID NO:83. The amino acid sequence of the HuD3 VL mini exon is SEQ ID NO:84. The nucleotide sequence encoding the HuD3 VH mini exon is SEQ ID NO:85. The amino acid sequence of the HuD3 VH mini exon is SEQ ID NO:86. The nucleotide sequences shown are flanked by MluI (ACGCGT) (SEQ ID NO:87) and XbaI (TCTAGA) (SEQ ID NO:88) sites. The signal peptide sequences are in italics. The CDRs based on the definition of Kabat are underlined. The mature light and heavy chains both begin with an alanine residue (double-underlined).

FIG. 24 depicts the binding of humanized and chimeric D3 antibodies to recombinant soluble human L-selectin. The ELISA experiment was carried out as described in Example 5.

FIG. 25 depicts the binding specificity of humanized and chimeric D3 antibodies. The FACS experiments using CHO transfectants expressing human L-selectin, E-selectin, or P-selectin were carried out as described in Example 5.

FIG. 26 depicts the affinities of HuD3 and ChD3 to L-selectin. The binding of biotinylated ChD3 to recombinant soluble human L-selectin analyzed by ELISA in the presence of different amounts of competitor antibody (HuD3, ChD3, HuDREG200, or Hu1D10) was performed as described in Example 5.

### DETAILED DESCRIPTION OF THE PREFERRED EMBODIMENTS

Definitions

In order that the invention may be more completely understood, several definitions are set forth. As used herein, the term “immunoglobulin” or “antibody” refers to a protein consisting of one or more polypeptides substantially encoded by immunoglobulin genes. The recognized immunoglobulin genes include the kappa, lambda, alpha, gamma (γ1, γ2, γ3, γ4), delta, epsilon, and mu constant region genes, as well as the myriad immunoglobulin variable region genes. Full-length immunoglobulin “light chains” (about 25 Kd or 214 amino acids) are encoded by a kappa or lambda variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a heavy chain variable region gene (about 116 amino acids) and one of the other aforementioned constant region genes, e.g., gamma (encoding about 330 amino acids).

One form of immunoglobulin constitutes the basic structural unit of an antibody. This form is a tetramer and consists of two identical pairs of immunoglobulin chains, each pair having one light and one heavy chain. In each pair, the light and heavy chain variable regions are together responsible for binding to an antigen, and the constant regions are responsible for the antibody effector functions. In addition to tetrameric antibodies, immunoglobulins may exist in a variety of other forms including, for example, Fv, Fab, and (Fab′)₂, as well as bifunctional hybrid antibodies (e.g., Lanzavecchia and Scheidegger, Eur. J. Immunol. 17:105-111 (1987)) and in single chains (e.g., Huston et al., Proc. Natl. Acad. Sci. USA, 85:5879-5883 (1988), and Bird et al., Science, 242:423-426 (1988), which are incorporated herein by reference). (See, generally, Hood et al., “Immunology”, 2^{nd} ed., Benjamin, New York (1984), and Hunkapiller and Hood, Nature, 323:15-16 (1986), which are incorporated herein by reference).

The term “substantially identical,” in the context of two nucleic acids or polypeptides (e.g., DNAs encoding a humanized immunoglobulin or the amino acid sequence of the humanized immunoglobulin) refers to two or more sequences or subsequences that have at least about 85%, most preferably 90 -95% or higher nucleotide or amino acid residue identity, when compared and aligned for maximum correspondence, as measured using the following sequence comparison method and/or by visual inspection. Such “substantially identical” sequences are typically considered to be homologous. Preferably, the “substantial identity” exists over a region of the sequences that is preferably about 50 residues in length, more preferably over a region of at least about 100 residues, and most preferably the sequences are substantially identical over at least about 150 residues, or over the full length of the two sequences to be compared. As described below, any two antibody sequences can only be aligned in one way, by using the numbering scheme in Kabat (“Sequences of Proteins of Immunological Interest” Kabat, E. et al., U.S. Department of Health and Human Service (1983)). Therefore, for antibodies, percent identity has a unique and well-defined meaning.

Methods of determining percent identity are known in the art. “Percent (%) sequence identity” with respect to a specified subject sequence, or a specified portion thereof, may be defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the Smith Waterman algorithm (Smith & Waterman, J. Mol. Biol. 147 147:195-7 (1981)) using the BLOSUM substitution matrices (Henikoff& Henikoff, Pros. Natl. Acad. Sci. USA 89:10915-9 (1992)) as similarity measures. A “% identity value” is determined by the number of matching identical nucleotides or amino acids divided by the sequence length for which the percent identity is being reported.

As used herein, the term “framework region” refers to those portions of immunoglobulin light and heavy chain variable regions that are relatively conserved (i.e., other than the CDRs) among different immunoglobulins in a single species, as defined by Kabat, et al. As used herein, a “human framework region” is a framework region that is substantially identical to the framework region of a naturally occurring human antibody.

From N-terminal to C-terminal, both light and heavy chain variable regions comprise alternating frameworks (FR) and complementarity determining regions (CDRs): FR, CDR, FR, CDR, FR, CDR and FR. The assignment of amino acids to each region is in accordance with the definitions of Kabat, and/or Chothia & Lesk, J. Mol. Biol. 196:901-917 (1987); Chothia et al., Nature 342:878-883 (1989).

Amino acids from the variable regions of the mature heavy and light chains of immunoglobulins are designated Hx and Lx respectively, where x is a number designating the position of an amino acid according to the scheme of Kabat, Sequences of Proteins of Immunological Interest (National Institutes of Health, Bethesda, Md., 1987 and 1991). Kabat lists many amino acid sequences for antibodies for each subgroup, and lists the most commonly occurring amino acid for each residue position in that subgroup to generate a consensus sequence.

Kabat uses a method for assigning a residue number to each amino acid in a listed sequence, and this method for assigning residue numbers has become standard in the field. Kabat's scheme is extendible to other antibodies not included in his compendium by aligning the antibody in question with one of the consensus sequences in Kabat by reference to conserved amino acids. That is, the heavy and light chains of an antibody are aligned with the heavy and light chains of EU to maximize amino acid sequence identity and each amino acid in the antibody is assigned the same number as the corresponding amino acid in EU. The use of the Kabat numbering system readily identifies amino acids at equivalent positions in different antibodies. For example, an amino acid at the L20 position of a human antibody occupies the equivalent position to an amino acid position L20 of a chicken antibody.

Furthermore, for the unambiguous identification of corresponding residues of two species′ framework regions, it may be useful to separately consider the individual framework regions (i.e., as separated by the CDRs) and to designate particular residues with respect to an individual framework region. Using this system, a particular residue is designated as occupying the nth position in the first, second, third, or fourth framework region. For residues in the second, third, or fourth framework region, counting begins at “1”after the previous CDR. For residues in the first framework region, counting begins at “1”at the beginning of the variable region.

As used herein, the term “humanized immunoglobulin” refers to an immunoglobulin comprising a human framework, at least one CDR from a non-human antibody, and in which any constant region present is substantially identical to a human immunoglobulin constant region, i.e., at least about 85%, preferably at least 90-95% identical. Hence, all parts of a humanized immunoglobulin, except possibly the CDRs, are substantially identical to corresponding parts of one or more native human immunoglobulin sequences. A. “humanized antibody” is an antibody comprising humanized light chains and humanized heavy chains.

Preferably, analogs of exemplified humanized immunoglobulins differ from exemplified immunoglobulins by conservative amino acid substitutions. For purposes of classifying amino acids substitutions as conservative or nonconservative, amino acids may be grouped as follows: Group I (hydrophobic sidechains): Met, Ala, Val, Leu, Ile; Group II (neutral hydrophilic side chains): Cys, Ser, Thr; Group III (acidic side chains): Asp, Glu; Group IV (basic side chains): Asn, Gln, His, Lys, Arg; Group V (residues influencing chain orientation): Gly, Pro; and Group VI (aromatic side chains): Trp, Tyr, Phe. Conservative substitutions involve substitutions between amino acids in the same class. Non-conservative substitutions constitute exchanging a member of one of these classes for a member of another.

The term “chimeric antibody” refers to an antibody in which the constant region comes from an antibody of one species (typically human) and the variable region comes from an antibody of another species (typically non-human vertebrate). Such antibodies retain the binding specificity of the non-human vertebrate antibody, while being about two-thirds human.

The term “derived from” means “obtained from” or “produced by”.

The term “epitope” includes any protein portion capable of specific binding to an immunoglobulin or an antibody. Epitopic determinants usually consist of active surface groupings of molecules such as amino acids or sugar side chains and usually have specific three-dimensional structural characteristics, as well as specific charge characteristics. Two antibodies are said to bind to the same epitope of a protein if amino acid mutations in the protein that reduce or eliminate binding of one antibody also reduce or eliminate binding of the other antibody, and/or if the antibodies compete for binding to the protein, i.e., binding of one antibody to the protein reduces or eliminates binding of the other antibody.

The term “substantially pure” or “isolated” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition comprises more than about 80, 90, 95 or 99% percent by weight of all macromolecular species present in the composition. Most preferably, the object species is purified to essential homogeneity (contaminant species cannot be detected in the composition by conventional detection methods) wherein the composition consists essentially of a single macromolecular species.

By “a therapeutically effective” amount of a drug or pharmacologically active agent or pharmaceutical formulation is meant a sufficient amount of the drug, agent or formulation to provide the desired effect.

A “subject” or “patient” is used interchangeably herein, which refers to a vertebrate, preferably a mammal, more preferably a human.

I. Chicken Anti-IL-12 Monoclonal Antibodies and Chimeric Antibodies

In order to generate humanized chicken antibodies, chicken monoclonal antibodies against a human protein such as IL-12 are isolated. The present invention provides an isolated chicken antibody (monoclonal or polyclonal) that recognizes IL-12 derived from any species and origin, preferably human or mouse IL-1 2. Preferably, the chicken antibodies bind to IL-12 of multiple mammals such as human and mouse. The chicken antibodies may bind to any epitope or subunit of IL-12, or neutralize at least biological activities of IL-12. Preferably, the chicken antibodies bind to at least one of the epitopes presented by the three dimensional conformation of the IL-12 p75 heterodimer.

In one embodiment, the chicken anti-IL-12 antibody includes, but is not limited to, a chicken anti-human and/or mouse IL-12 antibody, which comprises a heavy chain variable region as presented in SEQ ID NO: 2 and a light chain variable region as presented in SEQ ID NO: 4. The polynucleotides encoding the chicken antibody are also included. An exemplary polynucleotide encoding the heavy chain variable region is presented in SEQ ID NO: 1 and the polynucleotide encoding light chain variable region is presented in SEQ ID NO: 3.

In another embodiment, the chicken anti-IL-12 antibody includes, but is not limited to, a chicken anti-human and/or mouse IL-12 antibody, which comprises a heavy chain variable region as presented in SEQ ID NO: 48 and a light chain variable region as presented in SEQ ID NO: 47. The polynucleotides encoding the chicken antibody are also included. An exemplary polynucleotide encoding the heavy chain variable region is presented in SEQ ID NO: 73 and the polynucleotide encoding light chain variable region is presented in SEQ ID NO: 71.

Chicken anti-IL-12 monoclonal antibodies can be isolated by using phage display methodology, which is known in the art (Davies, E. et al., Journal of Immunological Methods 186: 125-135 (1995); Yamanaka, H. et al., Journal of Immunology 157: 1156-1162 (1996)), each of which is incorporated by reference in its entirety). In particular, chicken is immunized with the human or/and mouse IL-12 protein following procedures known in the art. The spleen of the immunized chicken is then harvested and total RNA is isolated. A phage display library for expression of chicken antibodies is constructed (see more details in the Examples). Phage clones are selected based on the binding of the expressed antibody to IL-12. The DNAs encoding the expressed chicken antibodies of the selected phage clone are subcloned into approximate expression vectors and transfected into mammalian host cells, such as myeloma cell line, and the desired chicken antibodies are so produced.

Alternatively, chicken monoclonal antibodies can be produced by using hybridoma fusion methodology known in the art (Matsuda, H. et al., FEMS Immunology and Medical Microbiology 23: 189-194 (1999); Nishinaka, S. et al., J. Vet. Med. Sci. 58(11): 1053-1056 (1996); and European Patent Application EP 0737743A1, each of which is incorporated by reference in its entirety).

The present invention also includes modified anti-IL-12 chicken antibodies that are functionally equivalent to the natural chicken anti-IL-12 antibodies. Modified antibodies providing improved stability and/or therapeutic efficacy are preferred. Examples of modified antibodies include those with conservative substitutions of amino acid residues, and one or more deletions or additions of amino acids that do not significantly alter the antigen binding affinity. Substitutions can range from changing or modifying one or more amino acid residues to complete redesign of a region as long as the therapeutic utility is maintained. Amino acid substitutions, if present, are preferably conservative substitutions that do not deleteriously affect folding or functional properties of the antibody. Antibodies of this invention can be modified post-translationally (e.g., acetylation, and phosphorylation) or synthetically (e.g., the attachment of a labeling group). Fragments of these modified antibodies are also included.

The present invention also provides a chimeric antibody comprising (a) a variable region capable of binding to human or/and mouse IL-12 and (b) a constant region of a separate antibody, wherein said variable region and said constant region are derived from different species. Preferably, the constant region is derived from human and the variable region is derived from an avian, preferably a chicken.

An exemplary chimeric antibody comprises two light chains and two heavy chains, each of said chains having a chicken variable region binds to and/or neutralize IL-12, preferably human or/and mouse IL-12, and a human constant region, preferably IgG1/λ. More preferably, the present invention provides a chimeric chicken antibody comprising: (a) a heavy chain variable region as presented in SEQ ID NO:2 or SEQ ID NO:48; (b) a light chain variable region as presented in SEQ ID NO:4 or SEQ ID NO:47; and (c) human γ1 heavy chain and human λ light chain constant regions.

The heavy chains of the chimeric antibodies can have constant regions selected from any of the five isotypes alpha, delta, epsilon, gamma or mu. In addition, heavy chains may be of various subclasses (such as the IgG subclasses). The different classes and subclasses of heavy chains provide for different effector functions and, thus by choosing the desired heavy chain constant region, a chimeric antibody with a desired effector function can be produced. The light chains can have either a kappa or lambda constant chain.

In order to produce the above-mentioned chimeric antibody, the portions derived from two different species (e.g., human constant region and avian, and preferably chicken, variable or binding region) can be joined together chemically by conventional techniques or can be prepared as single contiguous proteins using genetic engineering techniques. Polynucleotide molecules encoding the proteins of both the light chain and heavy chain portions of the chimeric antibody can be expressed as contiguous proteins in a host expression system, which will be discussed later in detail. The method of making the chimeric antibody is disclosed in U.S. Pat. Nos. 5,677,427; 6,120,767; and 6,329,508, each of which is incorporated by reference in its entirety.

II. Chicken Anti-L-selectin Antibodies

The present invention also provides a chicken immunoglobulin that specifically binds to an antigen or epitope, wherein the antigen or epitope is specifically found in mammals. Preferably, the antigen or epitope is found in a mammalian protein. More preferably, the mammalian protein is found in more than one mammalian species. Preferably, the antigen or epitope is one that is found in more than one mammalian species. Preferably, the antigen or epitope is common to more than one mammalian protein within a mammalian species.

Preferably, the antigen is a conserved antigen found in multiple, functionally-related proteins. Preferably, the multiple, functionally-related proteins share substantially sequence similarity or homology and belong to a protein family. More preferably, the protein family is the selectin protein family, and the multiple, functionally-related, proteins are E-selectin, P-selectin and L-selectin.

The present invention provides an isolated chicken antibody that recognizes multiple, functionally-related proteins that share substantially sequence similarity or homology and belong to a protein family. Preferably, the proteins are found a mammalian species, such as human or mouse. Preferably, the chicken antibodies bind to the multiple, functionally-related proteins of multiple mammalians, such as human and mouse.

Preferably, the protein family includes, but is not limited to, selectins, integrins, cadherins, chemokines, chemokine receptors, ion channels, ephrins, ephrin receptors, frizzleds, WNTs, metallothioneins, matrix metalloproteinases, epidermal growth factors (EGF), EGF receptors, fibroblast growth factors (FGF), FGF receptors, nerve growth factor (NGF), and NGF receptors. Chicken antibodies capable of binding to one or more proteins within a protein family are especially useful treating one or more diseases, disorders or conditions that are caused or aggravated by the normal or increased biological activity of the proteins of the protein family.

More preferably, the protein family is selecting. When the protein family is selecting, the proteins bound by the chicken antibodies are preferably L-selectin, P-selectin, and/or E-selectin.

The chicken antibodies may bind to any epitope or subunit of the multiple, functionally-related proteins, or neutralize at least one or more biological activities of the multiple, functionally-related proteins.

In one embodiment, the chicken anti-L-selectin antibody includes, but is not limited to, a chicken anti-human and/or mouse L-selectin antibody, which comprises a heavy chain variable region as presented in SEQ ID NO: 82 and a light chain variable region as presented in SEQ ID NO: 80. The polynucleotides encoding the chicken antibody are also included. An exemplary polynucleotide encoding the heavy chain variable region is presented in SEQ ID NO: 85 and the polynucleotide encoding light chain variable region is presented in SEQ ID NO: 83.

Chicken anti-L-selectin monoclonal antibodies can be isolated using any of the methods disclosed in this application. In particular, chicken is immunized with the human or/and mouse L-selectin.

The present invention also includes modified anti-L-selectin chicken antibodies that are functionally equivalent to the natural chicken anti-L-selectin antibodies. Modified antibodies providing improved stability and/or therapeutic efficacy are preferred. Examples of modified antibodies include those with conservative substitutions of amino acid residues, and one or more deletions or additions of amino acids that do not significantly deleteriously alter the antigen binding utility. Substitutions can range from changing or modifying one or more amino acid residues to complete redesign of a region as long as the therapeutic utility is maintained. Amino acid substitutions, if present, are preferably conservative substitutions that do not deleteriously affect folding or functional properties of the antibody. Antibodies of this invention can be modified post-translationally (e.g., acetylation, and phosphorylation) or synthetically (e.g., the attachment of a labeling group). Fragments of these modified antibodies are also included.

The present invention also provides a chimeric antibody comprising (a) a variable region capable of binding to human or/and mouse L-selectin and (b) a constant region of a separate antibody, wherein said variable region and said constant region are derived from different species. Preferably, the constant region is derived from human and the variable region is derived from an avian, preferably a chicken.

An exemplary chimeric antibody comprises two light chains and two heavy chains, each of said chains having a chicken variable region binds to or neutralize L-selectin, preferably human or/and mouse L-selectin, and human constant regions, preferably IgG1/λ. More preferably, the present invention provides a chimeric chicken antibody comprising: (a) a heavy chain variable region as presented in SEQ ID NO:82; (b) a light chain variable region as presented in SEQ ID NO: 80; and (c) human γ1 heavy chain and human λ light chain constant regions.

The heavy chains of the chimeric antibodies can have constant regions selected from any of the five isotypes alpha, delta, epsilon, gamma or mu. In addition, heavy chains may be of various subclasses (such as the IgG subclasses). The different classes and subclasses of heavy chains provide for different antibody effector functions and, thus by choosing the desired heavy chain constant region, a chimeric antibody with a desired effector function can be produced. The light chains can have either a kappa or lambda constant chain.

In order to produce the above-mentioned chimeric antibody, the portions derived from two different species (e.g., human constant region and chicken variable or binding region) can be joined together chemically by conventional techniques or can be prepared as single contiguous proteins using genetic engineering techniques. Polynucleotide molecules encoding the proteins of both the light chain and heavy chain portions of the chimeric antibody can be expressed as contiguous proteins in a host expression system, which will be discussed later in detail. The method of making the chimeric antibody is disclosed in U.S. Pat. Nos. 5,677,427; 6,120,767; and 6,329,508, each of which is incorporated by reference in its entirety.

III. Humanized Chicken Antibodies

The present invention provides for a humanized immunoglobulin having at least one complementarity determining region (CDR) from a donor immunuglobulin and heavy chain and light chain frameworks from human acceptors. Preferably, the donor immunoglobulin comprises a lambda light chain. The donor is any chicken species.

In a preferred embodiment, the light chain and the heavy chain of the humanized antibody of this invention have the CDRs of the donor immunoglobulin. In other embodiments, one or more substitutions are made in the CDRs. Such substitutions are generally conservative substitutions that do not substantially reduce the binding affinity of the humanized antibody to its antigen, in comparison to the affinity of the chicken donor antibody. Such substitutions may also be non-conservative substitutions that enhance the binding affinity of the humanized antibody to its antigen, in comparison to the affinity of the chicken donor antibody.

Humanized immunoglobulins of the invention have variable framework regions substantially from human immunoglobulins (termed as acceptor region). In general, acceptor framework regions are chosen that are homologous to the donor framework from which the CDRs are derived. The heavy chain and light chain frameworks may be from the same human antibody or may be from different antibodies. The heavy chain and light chain frameworks may also be from human genomic V and J segments. The framework sequences may be derived from rearranged variable genes. The framework sequences may be derived from germline or genomic sequences, or from cDNA sequences. The framework regions may also represent a consensus derived from different framework regions. Many of the amino acids in the framework region make little or no direct contribution to the specificity or affinity of an antibody. Thus, many individual conservative substitutions of framework residues can be tolerated without appreciable change of the specificity or affinity of the resulting humanized immunoglobulin. However some positions in the framework may be crucial for the proper binding of CDRs to the antigen. Accordingly, those positions outside the CDRs are occupied by the amino acid residues of the chicken donor immunoglobulin. Identifying such positions is an important task in designing humanized chicken antibodies.

The design of humanized immunoglobulins can be carried out by following the guideline set forth in the present invention. In particular, when an amino acid falls under Category (a) to (c), the framework amino acid of a human immunoglobulin to be used (acceptor immunoglobulin) is replaced by a framework amino acid from a CDR-providing non-human immunoglobulin (donor immunoglobulin) or by a consensus human framework amino acid at the corresponding position:

(a) The amino acid is in a CDR defined by Kabat, et al. (Sequences of Proteins of Immunological Interest (National Institutes of Health, Bethesda, Md., 1991).

(b) The amino acid is capable of interacting with one of the CDRs; a three dimensional model, typically of the original donor antibody, shows that certain amino acids outside of the CDR's are close to the CDR's and have a good probability of interacting with amino acids in the CDR's by hydrogen bonding, Van der Waals forces, hydrophobic interactions, etc. At those amino acid positions, the donor immunoglobulin amino acid rather than the acceptor immunoglobulin amino acid may be selected. Amino acids according to this criterion will generally have a side chain atom within about 3, 5, 7, or 10 angstrom units of some atom in the CDR's and must contain an atom that could interact with the CDR atoms according to established chemical forces, such as those listed above. The amino acid that belongs to this category can be distinguished by analyzing the possible interactions with the CDRs in a three-dimensional computer model in a number of approaches, which are disclosed in detail in U.S. Pat. Nos. 6,180,370 and 5,585,089, each of which is herein incorporated by reference in its entirety;

(c) If an amino acid in the framework of the human acceptor immunoglobulin is unusual (i.e., “rare”, which as used herein indicates an amino acid occurring at that position in less than about 20% but usually less than about 10% of human heavy or light chain V region sequences in a representative data bank), the framework amino acid of human acceptor immunuglobulin is replaced by a consensus amino acid, which is the residue typical for the human sequence.

Inspection of the amino acid sequences of chicken and human immunoglobulins revealed several important heavy and light chain framework positions for the humanization design. In these positions, the amino acid residues are usually different between a chicken and a human. The framework residues in at least one of these positions of humanized chicken immunoglobulins are replaced.

Some of these positions interact with one CDR in most chicken antibodies. These positions include H67, H78, H93, L46, L66, and L69 (Kabat numbering). The residues in at least one of these positions of the humanized chicken immunoglobulins are replaced, and preferably, at least one of these positions are occupied by the amino acid residues in the equivalent positions of the chicken donor immunoglobulin frameworks.

In addition, no amino acid residues exist at positions L1 and L2 (Kabat numbering) of a chicken immunoglobulin. Amino acid residues of a human framework in L1 and L2 may be added to L1 and L2 of the humanized chicken immunoglobulin respectively.

Furthermore, an extra amino acid residue, typically a serine, is commonly found in chicken immunoglobulins between positions L39 and L40 (Kabat numbering), that is herein designated L39A. This extra residue may be deleted when human frameworks are used in the humanized immunoglobulins of the present invention. As examples of humanized chicken antibodies, the present invention provides humanized versions of the chicken monoclonal antibodies that bind to or neutralize IL-12. Preferably, said humanized chicken antibody binds to or neutralizes human or mouse IL-12, more preferably, the antibody binds to or neutralizes both mouse and human IL-12.

In one embodiment, the amino acid sequences of the heavy chain variable region and light chain variable region of a chicken donor immunoglobulin, chicken anti-IL-12 antibody B1 are provided in, respectively, SEQ ID NOs: 2 and 4.

In another embodiment, the amino acid sequences of the heavy chain variable region and light chain variable region of a chicken donor immunoglobulin, chicken anti-IL-12 antibody B1 are provided in, respectively, SEQ ID NOs: 48 and 47.

Preferably, the heavy and light chain framework regions are from human antibodies. In one embodiment, the framework of humanized chicken anti-IL-12 variable regions is designed as follows: First, a molecular model of the chicken donor variable regions is constructed with the aid of the computer programs including but not limited to ABMOD (Zilber, B., Scherf, T., Levitt, M., and Anglister, J. Biochemistry 29:10032-41 (1990)) and ENCAD (Levitt, M., J. Mol. Biol. 168: 595-620 (1983)). Next, a homology search is conducted between the chicken donor and human acceptor amino acid sequences to choose appropriate acceptor heavy and light chain frameworks. The selected acceptor immunoglobulin chain will most preferably have at least about 60% homology in the framework region to the donor immunoglobulin, preferably, about at least 70% homology. For example, the VL segment DPL16 (Williams, S.C. and Winter, G., Eur. J. Immunol. 23: 1456-1461 (1993)) and the J segment Jλ2 (Udey, J.A. and Blomberg, B., Immunogenetics 25: 63-70 (1987)) is selected to provide the frameworks for the humanized form of the light chain variable region of the chicken anti-IL-12 monoclonal antibody B1 (described in Examples 1, 2 and 3), and the VH segment DP-54 (Tomlinson, I.M., et al., J. Mol. Biol. 227: 776-798 (1992)) and the J segment JH1 (Ravetch, J.V., et al., Cell 27: 583-591 (1981)) for the humanized heavy chain variable region of the chicken anti-IL-12 monoclonal antibody B1 (described in Examples 1, 2 and 3). The identity of the framework amino acids between chicken light chain variable region of the anti-IL-12 antibody and the acceptor human DPL16 and Jλ2 segments is 70%. The identity between chicken heavy chain variable region and the human DP-54 and JH1 segments is 72%.

The heavy chain variable framework of the human DP-54 and JH1 segments is: H1-H30 presented in SEQ ID NO: 5, H36-H49 presented in SEQ ID NO: 6, H66-H94 presented in SEQ ID NO: 7, and H103-H113 presented in SEQ ID NO: 8. The light chain framework human DPL16 and Jλ2 segments is: L1-L23 presented in SEQ ID NO: 9, L35-L49 presented in SEQ ID NO: 10, L57-L88 presented in SEQ ID NO: 11, and L98-L107 presented in SEQ ID NO: 12.

Examples of framework positions that have significant contact with one of the CDRs include, but are not limited to, L46, L57, L60, L66 and L69 of the light chain and H47, H67, and H78 of the heavy chain. The humanized chicken anti-IL-12 immunoglobulin framework has at least one position selected from the group consisting of H47, H67, H78, L46, L57, L60, L66, and L69 that is occupied by an amino acid in the equivalent position of the chicken donor immunoglobulin.

In addition, human framework residues that are found to be rare in the same variable region subgroup are changed to the corresponding consensus amino acids to eliminate potential immunogenicity. For humanized chicken anti-IL-12 antibody B1, such residues include, but are not limited to, L7, L9, L72 and L78 in the light chain and H77 in the heavy chain. The humanized chicken immunoglobulin has at least one position selected from the group consisting of H77, L7, L9, L72 and L74 that is occupied by a consensus amino acid in the human acceptor immunoglobulin. Preferably, H77 is occupied by threonine, L7 is occupied by proline, L9 is occupied by serine, L72 is occupied by threonine, and L78 is occupied by valine.

Preferably, the humanized chicken immunoglobulin has H47, H67, H78, L46, L57, L60, L66, and L69 occupied by an amino acid in the equivalent position of the chicken donor immunoglobulin, and H77, L7, L9, L72 and L78 is occupied by a consensus amino acid in the human acceptor immunoglobulin.

In one embodiment, the amino acid sequence of the humanized (mature) heavy chain variable region with anti-IL-12 specificity is presented in SEQ ID NO: 14. The amino acid sequence of the humanized (mature) light chain variable region with anti-IL-12 specificity is presented in SEQ ID NO:16.

In another embodiment, the amino acid sequence of the humanized (mature) heavy chain variable region with anti-IL-12 specificity is presented in SEQ ID NO: 50. The amino acid sequence of the humanized (mature) light chain variable region with anti-IL-12 specificity is presented in SEQ ID NO:49.

In another embodiment, the amino acid sequence of the humanized (mature) heavy chain variable region with anti-L-selectin specificity is presented in SEQ ID NO: 82. The amino acid sequence of the humanized (mature) light chain variable region with anti-L-selectin specificity is presented in SEQ ID NO:80.

In a preferred embodiment, the heavy chain and light chain framework regions have at least 60%, more preferably at least 70% sequence identity to the human acceptor frameworks (the framework of human DP-54 and JH1 segments and human DPL16 and Jλ2 segments).

The variable segments of humanized antibodies produced are typically linked to at least a portion of immunoglobulin constant regions, typically that of a human immunoglobulin. Human constant region DNA sequences can be isolated in accordance with well-known procedures from a variety of human cells, but preferably immortalized B-cells (see Kabat et al., supra, and WO87/02671). Ordinarily, the antibody contains both light chain and heavy chain constant regions. The heavy chain constant region usually includes CH1, hinge, CH2, CH3, and, sometimes, CH4 regions.

The humanized chicken antibodies of the present invention include antibodies having all types of constant regions, including IgM, IgG, IgD, IgA and IgE, and any isotype, including IgG1, IgG2, IgG3 and IgG4. When it is desired that the humanized antibody exhibit cytotoxic activity through the activation of human effector cells, including peripheral mononuclear cells, monocytes, and granulocytes, antibodies of the IgG1 and IgG3 subclasses may be preferred. When such cytotoxic activity is not desirable, the constant domain can be of the IgG2 or IgG4 subclass. The humanized antibody may comprise sequences from more than one class or isotype.

In one embodiment, the present invention is directed to recombinant polynucleotides that encode the mature heavy and light chains of humanized anti-IL-12 antibodies or humanized anti-L-selectin antibodies, as well as the heavy and/or light chain CDRs from the chicken donor antibody. Because of the degeneracy of the genetic code, a variety of nucleic acid sequences encode each immunoglobulin amino acid sequence. The desired nucleic acid sequences can be produced by de novo solid-phase DNA synthesis or by PCR mutagenesis of an earlier prepared variant of the desired polynucleotide. In a preferred embodiment, the codons that are used comprise those that are typical for human (see, e.g., Nakamura, Y., Nucleic Acids Res. 28: 292 (2000)).

In one embodiment, the polynucleotide sequence encoding the mature anti-IL-12 heavy chain variable region of SEQ ID NO:14 is provided in SEQ ID NO:13. An exemplary polynucleotide sequence encoding the mature anti-IL-12 light chain variable region of SEQ ID NO: 16 is provided in SEQ ID NO:15.

In another embodiment, the polynucleotide sequence encoding the mature anti-IL-12 heavy chain variable region of SEQ ID NO:50 is provided in SEQ ID NO:73. An exemplary polynucleotide sequence encoding the mature anti-IL-12 light chain variable region of SEQ ID NO: 49 is provided in SEQ ID NO: 71.

In another embodiment, the polynucleotide sequence encoding the mature anti-L-selectin heavy chain variable region of SEQ ID NO:82 is provided in SEQ ID NO:85. An exemplary polynucleotide sequence encoding the mature anti-L-selectin light chain variable region of SEQ ID NO:80 is provided in SEQ ID NO:83.

The polynucleotides of this invention also include expression vectors for recombinant expression of the humanized immunoglobulins.

In one embodiment, the humanized antibodies are encoded by nucleic acid sequences that hybridize with the nucleic acids encoding the heavy or light chain variable regions of humanized chicken antibody or degenerate forms thereof, under stringent conditions. Phage-display technology offers powerful techniques for selecting such analogs of humanized chicken antibody with retaining binding affinity and specificity (see, e.g., Dower et al., WO 91/17271; McCafferty et al., WO 92/01047; and Huse, WO 92/06204). In one example, a phage display technique uses random mutation of framework region residues (Baca, M. et al., J. Biol. Chem. 272: 10678 (1997); W09845332).

The humanized antibodies of this invention exhibit a specific binding affinity for its antigen of at least 10⁷, 10⁸, 10⁹, or 10¹⁰ M⁻¹. Usually the upper limit of binding affinity of the humanized antibodies for the antigen is within a factor of 2, 3, 4, 5 or 10 of that of chicken donor antibodies. Often the lower limit of binding affinity is also within a factor of 2, 3, 4, 5 or 10 of that of chicken donor antibodies. Affinity determinations, including association and dissociation constants, may be made using any available method, such as by surface plasmon resonance using a Biacore instrument (Biacore AB, Uppsala, Sweden), by quantitative ELISA, competition ELISA, radioimmunoassay or FACS analysis, all of which are well known in the art.

The humanized chicken antibodies of the present invention have the similar binding characteristics and/or neutralizing abilities as their chicken donor immunoglobulins and are capable of binding to a protein (antigen) derived from multiple mammalian species, such as human and rodents. In one exemplary embodiment, the humanized chicken antibodies are capable of binding to a first antigen derived from a human and a second antigen derived from a non-human mammal, wherein said second antigen is substantially identical to the first antigen. Preferably, said non-human animal is a mouse or rat.

In one embodiment, preferred humanized chicken immunoglobulins compete with chicken donor antibodies for binding to their antigens, for example IL-12, and prevent their antigens from binding to and thereby transducing a response through the signaling pathway. In one example, the humanized anti-IL-12 antibodies preferably neutralize at least 80, 90, 95 or 99% of human and/or mouse IL-12 activity at 1-, 2-, 5-, 10-, 20-, 50- or 100-fold molar excess. In a typical example, neutralizing activity is determined by the ability of the humanized chicken antibody to compete with an appropriately labeled chicken donor antibody, e.g., biotinylated and radio-labeled antibodies, for binding to the IL-12 protein or peptide.

In another embodiment, preferred humanized chicken immunoglobulins compete with chicken donor antibodies for binding to their antigens, for example L-selectin, and prevent their antigens from binding to and thereby transducing a response through the signaling pathway. In one example, the humanized anti-L-selectin antibodies preferably neutralize at least 80, 90, 95 or 99% of human and/or mouse L-selectin activity at 1-, 2-, 5-, 10-, 20-, 50- or 100-fold molar excess. In a typical example, neutralizing activity is determined by the ability of the humanized chicken antibody to compete with an appropriately labeled chicken donor antibody, e.g., biotinylated and radio-labeled antibodies, for binding to the L-selectin protein or peptide.

In one embodiment, the present invention is directed to recombinant polynucleotides that encode the mature heavy and light chains of humanized anti-L-selectin antibodies, as well as the heavy and/or light chain CDRs from the chicken donor antibody. Because of the degeneracy of the genetic code, a variety of nucleic acid sequences encode each immunoglobulin amino acid sequence. The desired nucleic acid sequences can be produced by de novo solid-phase DNA synthesis or by PCR mutagenesis of an earlier prepared variant of the desired polynucleotide. In a preferred embodiment, the codons that are used comprise those that are typical for human (see, e.g., Nakamura, Y., Nucleic Acids Res. 28: 292 (2000)).

In one embodiment, the polynucleotide sequence encoding the mature anti-L-selectin heavy chain variable region of SEQ ID NO: 82 is provided in SEQ ID NO: 85. An exemplary polynucleotide sequence encoding the mature anti-L-selectin light chain variable region of SEQ ID NO: 80 is provided in SEQ ID NO: 83.

For certain applications, it may be desirable to use antibodies that lack part or all of the constant regions. It may be usefull to use Fab, F(ab′)₂, Fv or single chain antibodies. Accordingly, polypeptide fragments comprising only a portion of the primary antibody structure may be produced. The fragments typically possess one or more immunoglobulin activities. These polypeptide fragments may be produced by proteolytic cleavage of intact antibodies by methods well known in the art, or by inserting stop codons at the desired locations in the DNA encoding the heavy chain of an antibody using site-directed mutagenesis, such as after CH1 to produce Fab fragments or after the hinge region to produce (Fab′)₂ fragments. Single chain antibodies may be produced by joining V_{L} and VH with a peptide linker (Bird, et al., Science 242:423-426 (1988); Huston, et al., Proc. Natl. Acad. Sci. U.S.A. 85:5879-5883 (1998), each of which is incorporated by reference in its entirety). Also because the immunoglobulin-related genes, like many other genes, contain separate functional regions, each having one or more distinct biological activities, the genes may be fused to functional regions from other genes (e.g., enzymes, see, commonly assigned U.S. Pat. No. 5,004,692) to produce fusion proteins (e.g., immunotoxins) having novel properties. The nucleic acid sequences of the present invention capable of ultimately expressing the desired humanized antibodies can be formed from a variety of different polynucleotides (genomic or cDNA, RNA, synthetic oligonucleotides, etc.) and components (e.g., V, J, D, and C regions), as well as by a variety of different techniques. Joining appropriate synthetic and genomic sequences is presently the most common method of production, but cDNA sequences may also be utilized (European Pat. Publication No. 0239400 and Reichmann, L. et al., Nature, 332: 323-327 (1988), both of which are incorporated herein by reference).

The present invention provides a polypeptide comprising an amino acid sequence of SEQ ID NO: 2, 4, 14, 16, 47, 48, 49, 50, 72, 74, 76, 78, 79, 80, 81, 82, 84, or 86. The present invention also provides a polynucleotide encoding an amino acid sequence of SEQ ID NO: 2, 4, 14, 16, 47, 48, 49, 50, 72, 74, 76, 78, 79, 80, 81, 82, 84, or 86. In some embodiments, the polypeptide is a fragment of the chicken monoclonal antibodies and its humanized versions described herein, such as a partial or full light or heavy chain, preferably, the heavy or light chain variable regions.

The present invention provides a method of producing a humanized chicken immunoglobulin comprising:

(a) preparing vectors comprising DNA segments encoding heavy and/or light chain variable regions of the humanized immunoglobulin having complementarity determining regions (CDRs) from a donor immunoglobulin and heavy and light chain variable region frameworks from human acceptor immunoglobulins, wherein said donor immunoglobulin is a chicken immunoglobulin; (b) transforming host cells with said vectors; and (c) culturing said transformed host cells to produce said humanized immunoglobulin.

In one preferred embodiment, the humanized immunoglobulin of the above method comprises amino acids from the donor immunoglobulin framework outside the CDRs of the humanized immunoglobulin that replace the corresponding amino acids in the acceptor immunoglobulin heavy or light chain frameworks, and each of these said donor amino acids are capable of interacting with the CDRs. In addition, the framework amino acid of human acceptor immunuglobulin of the above method may be replaced by a consensus amino acid when the amino acid residue is rare in human immunuglobulin sequences.

In another preferred embodiment, the heavy and light chain variable region frameworks of the humanized immunoglobulin of the above method have the residues in least one position selected from the group consisting of H67, H78, H93, L46, L66, L69 replaced, and preferably, at least one of these positions are occupied by the amino acid residues in the equivalent positions of the chicken donor immunoglobulin.

In one embodiment, the DNA segments or sequences encoding heavy and/or light chain variable regions or variable region frameworks are obtained or derived from germline or genomic sequences.

In another embodiment, the DNA segments or sequences encoding heavy and/or light chain variable regions or variable region frameworks are obtained or derived from cDNA sequences. Preferably, the cDNA sequences are as close as possible to the germline or genomic sequences.

Recombinant DNA techniques can be used to produce the chimeric and humanized antibodies and the fragments or conjugate thereof in any expression systems including both prokaryotic and eukaryotic expression systems. The DNA sequences will be expressed in host cells after the sequences have been operably linked to (i.e., positioned to ensure the functioning of) an expression control sequence. These expression vectors are typically replicable in the host organisms either as episomes or as an integral part of the host chromosomal DNA. Commonly, expression vectors will contain selection markers, e.g., tetracycline or neomycin, to permit detection of those cells transformed with the desired DNA sequences (U.S. Pat. No. 4,704,362, which is incorporated herein by reference in its entirety).

Expression vectors comprising the polynucleotides encoding the desired antibodies are delivered into host cells using conventional techniques known in the art. The desired antibodies are then expressed in the host cell.

Suitable host cells for the expression of the antibodies described herein are derived from prokaryote organism such as E. coli. or eukaryote organisms, including yeasts, plants, insects, and mammals.

E. coli is one prokaryotic host useful particularly for cloning and/or expressing DNA sequences of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters can be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation.

Other microbes, such as yeast, can also be used for expression. Saccharomyces is a preferred host, with suitable vectors having expression control sequences, such as promoters, including 3-phosphoglycerate kinase or other glycolytic enzymes, and an origin of replication, termination sequences and the like as desired.

Plants and plant cell cultures can be used for expression of the DNA sequence of the invention. (Larrick, et al., Hum. Antibodies Hybridomas 2(4): 172-89 (1991); Benvenuto, et al., Plant Mol. Biol. 17(4): 865-74 (1991); Durin, et al., Plant Mol. Biol. 15(2): 281-93 (1990); Hiatt, et al., Nature 342: 76-8 (1989), incorporated herein by reference in their entirety). Preferable plant hosts include, for example: Arabidopsis, Nicotiana tabacum, Nicotiana rustica, and Solanumtuberosum. A preferred expression cassette for expressing polynucleotide sequences encoding the antibodies of the invention is the plasmid pMOG18 in which the inserted polynucleotide sequence encoding the modified antibody is operably linked to a CaMV 35S promoter with a duplicated enhancer; pMOG18 is used according to the method of Sijmons, et al., Bio/Technology 8: 217-221 (1990), incorporated herein by reference in its entirety. Alternatively, a preferred embodiment. for the expression of the antibodies in plants follows the methods of Hiatt, et al., supra, with the substitution of polynucleotide sequences encoding the antibodies of the invention for the immunoglobulin sequences used by Hiatt, et al., supra. Agrobacterium tumifaciens T-DNA-based vectors can also be used for expressing the DNA sequences of the present invention, preferably such vectors include a marker gene encoding spectinomycin-resistance or other selectable marker.

Insect cell culture can also be used to produce the antibodies of the invention, typically using a baculovirus-based expression system. The antibodies can be produced by expressing polynucleotide sequences encoding the antibodies according to the methods of Putlitz, et al., Bio/Technology 8: 651-654 (1990), incorporated herein by reference in its entirety.

In addition to microorganisms and plants, mammalian tissue cell culture can also be used to express and produce the polypeptides of the present invention (see Winnacker, From Genes to Clones (VCH Publishers, NY, 1987), which is incorporated herein by reference in its entirety). Mammalian cells are actually preferred, because a number of suitable host cell lines capable of secreting intact immunoglobulins have been developed in the art, and include the CHO cell lines, various COS cell lines, HeLa cells, preferably myeloma cell lines, etc., or transformed B-cells or hybridomas. Expression vectors for these cells can include expression control sequences, such as an origin of replication, a promoter, an enhancer (Queen, et al., Immunol. Rev. 89: 49-68 (1986), which is incorporated herein by reference in its entirety), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. Generally, a selectable marker, such as a neoR expression cassette, is included in the expression vector.

The present invention includes polynucleotides encoding the antibodies and antibodies fragments described herein, including, but not limited to, a polynucleotide molecule comprising SEQ ID NO: 1, SEQ ID NO: 3, SEQ ID NO: 13 or SEQ ID NO: 15.

The present invention includes vectors comprising polynucleotides encoding the antibodies and antibodies fragments described herein.

The present invention includes host cells comprising the vectors comprising polynucleotides encoding the antibodies and antibodies fragments described herein.

Once expressed, the whole antibodies, their dimers, individual light and heavy chains, or other immunoglobulin forms of the present invention can be purified according to standard procedures of the art, including ammonium sulfate precipitation, affinity columns, column chromatography, gel electrophoresis and the like (Scopes, R., Protein Purification (Springer-Verlag, N.Y., 1982)). Substantially pure immunoglobulins of at least about 90 to 95% homogeneity are preferred, and 98 to 99% or more homogeneity most preferred, for pharmaceutical uses. Once purified, partially or to homogeneity as desired, the polypeptides may then be used therapeutically (including extra corporeally) or in developing and performing assay procedures, immunofluorescent stainings, and the like. (Immunological Methods, Vols. I and II (Lefkovits and Pernis, eds., Academic Press, NY, 1979 and 1981).

IV. Therapeutic and Diagnostic Applications The method of producing humanized chicken antibodies of the present invention can be used to humanize a variety of donor chicken antibodies, especially monoclonal antibodies reactive with markers on cells or soluble antigens responsible for a disease. Examples of such monoclonal antibodies include, but are not limited to, antibodies recognizing a viral surface protein, antibodies recognizing a tumor-related antigen, or suitable antibodies that bind to antigens on T-cells, such as those grouped into the so-called “Clusters of Differentiation,” as named by the First International Leukocyte Differentiation Workshop, Leukocyte Typing, Bernard, et al., Eds., Springer-Verlag, N.Y. (1984), which is incorporated herein by reference.

The produced humanized antibodies of the present invention are used in treating substantially any disease susceptible to monoclonal antibody-based therapy, depending on the antigen recognized by such antibodies. Typically, the antibodies can be used for passive immunization or the removal of unwanted cells or antigens, such as by complement mediated lysis, all without substantial immune reactions (e.g., anaphylactic shock) associated with many prior antibodies.

The humanized chicken antibodies are capable of binding to or neutralizing highly conserved proteins in mammals, which often play significant roles in numerous human biological pathways. Therefore, the antibodies of the present invention open a new avenue for human disease treatment. The antibodies binding to the antigen derived from multiple mammals such as human and rodent are particularly of great value in the evaluation of therapeutic values of those antibodies since the same antibodies can be used both in animal disease models and human clinical studies.

The humanized antibodies of the present invention can be used for the treatment of cancer (where the antibodies recognize a tumor-related antigen), viral infection (where the antibodies recognize viral surface proteins), autoimmune diseases, and prevention of tissue rejection in organ transplant (where the antibodies recognize the antigens on T-cells), etc. Examples of cancer include, but are not limited to, leukemias, lymphomas, sarcomas and carcinomas including tumors of the breast, colon, lung, prostate, pancreas and other organs. Examples of autoimmune diseases include, but are not limited to, Addison's disease, autoimmune diseases of the ear, autoimmune diseases of the eye such as uveitis, autoimmune hepatitis, Crohn's disease, diabetes (Type I), epididymitis, glomerulonephritis, Graves' disease, Guillain-Barre syndrome, Hashimoto's disease, hemolytic anemia, systemic lupus erythematosus, multiple sclerosis, myasthenia gravis, pemphigus vulgaris, psoriasis, rheumatoid arthritis, sarcoidosis, scleroderma, Sjogren's syndrome, spondyloarthropathies, thyroiditis, ulcerative colitis and vasculitis.

In one embodiment of the present invention, the anti-IL-12 antibodies of the present invention, preferably, humanized chicken antibodies, are useful antagonists for controlling diseases with pathologies that are mediated through immune mechanisms, particularly, diseases associated with increased IL-12 bioactivity that results in aberrant Thl-type helper cell activity. In accordance with the present invention, the anti-IL-12 antibodies are used for treating autoimmune disorders in humans or other mammals, such as, for example, psoriasis, multiple sclerosis, rheumatoid arthritis, autoimmune diabetes mellitus, and inflammatory bowel disease (IBD) including Crohn's disease and ulcerative colitis. The antibodies described herein can also be used to treat other disease conditions which have been shown to benefit from the administration of anti-IL-12 antibodies including, for example, transplantation/graft-versus-host disease and septic shock. The therapeutic and non-therapeutic use of the antibodies recognizing IL-12 (Natural Killer Stimulatory Factor; or Cytotoxic lymphocyte Maturation Factor) have been disclosed in more detail in U.S. Pat. Nos. 5,648,467; 5,811,523; 5,780,597; 6,300,478; and 6,410,824, each of which is incorporated by reference in its entirety.)

The present invention provides a pharmaceutical composition comprising antibodies, antibody fragments, and antibody conjugates described herein. The pharmaceutical composition can further comprise a pharmaceutical carrier.

The present invention provides a method of treating an autoimmune disease by administering the above pharmaceutical composition in a subject in need of such a treatment in a therapeutically effective amount. Preferably, said autoimmune disease is psoriasis.

The pharmaceutical composition of the present invention may also comprise the use of the subject antibodies in immunotoxins. Conjugates that are immunotoxins including conventional antibodies have been widely described in the art. The toxins may be coupled to the antibodies by conventional coupling techniques or immunotoxins containing protein toxin portions can be produced as fusion proteins. The conjugates of the present invention can be used in a corresponding way to obtain such immunotoxins. Illustrative of such immunotoxins are those described by Byers, B. S. et al. Seminars Cell Biol. 2: 59-70 (1991) and by Fanger, M. W. et al. Immunol. Today 12: 51-54 (1991).

Therapeutic methods are usually applied to human patients but may be applied to other non-human mammals.

There are various methods of administering the antibodies. The antibody may be administered to a patient intravenously as a bolus or by continuous infusion over a period of time, by intramuscular, intraperitoneal, intra-cerebrospinal, subcutaneous, intra-articular, intrasynovial, intrathecal, oral, topical, inhalation routes, or other delivery means known to those skilled in the art.

The pharmaceutical compositions of the present invention commonly comprise a solution of antibodies, or a cocktail thereof dissolved in an acceptable carrier, preferably an aqueous carrier. That is, the antibodies can be used in the manufacture of a medicament for treatment of cancer patients. A variety of aqueous carriers can be used, e.g., water for injection (WFI), or water buffered with phosphate, citrate, acetate, etc. to a pH typically of 5.0 to 8.0, most often 6.0 to 7.0, and/or containing salts such as sodium chloride, potassium chloride, etc. to make isotonic. The carrier can also contain excipients such as human serum albumin, polysorbate 80, sugars or amino acids to protect the active protein. The concentration of an antibody in these formulations varies widely from about 0.1 to 100 mg/ml but is often in the range 1 to 10 mg/ml. The formulated monoclonal antibody is particularly suitable for parenteral administration, and can be administered as an intravenous infusion or by subcutaneous, intramuscular or intravenous injection. Actual methods for preparing parentally administrable compositions are known or apparent to those skilled in the art and are described in more detail in, for example, Remington's Pharmaceutical Science (15th Ed., Mack Publishing Company, Easton, Pa., 1980), which is incorporated herein by reference.

The immunoglobulins of this invention can be frozen or lyophilized for storage and reconstituted in a suitable carrier prior to use. This technique has been shown to be effective with conventional immunoglobulins and art-known lyophilization and reconstitution techniques can be employed. Lyophilization and reconstitution can lead to varying degrees of immunoglobulin activity loss (e.g., with conventional immunoglobulins, IgM antibodies tend to have greater activity loss than IgG antibodies) and that use levels may have to be adjusted to compensate.

The compositions can be administered for prophylactic and/or therapeutic treatments. An amount adequate to accomplish the desired effect is defined as a “therapeutically effective dose” and will generally range from about 0.01 to about 100 mg of antibody per dose via single or multiple administrations. The amount of active ingredients that may be combined with the carrier materials to produce a single dosage form will vary depending upon the host treated and the particular mode of administration. It will be understood, however, that the specific dose level for any particular patient will depend upon a variety of factors, including the activity of the specific inhibitor employed, the age, body weight, general health, sex, diet, time of administration, route of administration, and rate of excretion, drug combination and the severity of the particular disease undergoing therapy, and can be determined by those skilled in the art.

When used therapeutically, the antibodies disclosed herein may be used in the unmodified form or may be modified with an effector moiety that delivers a toxic effect, such as a drug, cytotoxin (preferably, a protein cytotoxin or a Fc domain of the monoclonal antibodies), radionuclide, etc (see, e.g., U.S. Pat. No. 6,086,900). Additionally, the antibody can be utilized alone in substantially pure form, or together with other agents, as are known to those of skill in the art.

Antibodies disclosed herein are useful in diagnostic and prognostic evaluation of diseases and disorders, for example, detecting expression of the disease markers by using the methods known in the art, such as radioimmunoassay, ELISA, FACS, etc. One or more labeling moieties can be attached to the humanized immunoglobulin. Exemplary labeling moieties include radiopaque dyes, radiocontrast agents, fluorescent molecules, spin-labeled molecules, enzymes, or other labeling moieties of diagnostic value, particularly in radiologic or magnetic resonance imaging techniques. Methods of diagnosis can be performed in vitro using a cellular sample (e.g., blood sample, lymph node biopsy or tissue) from a patient or can be performed by in vivo imaging.

Kits can also be supplied for use with the modified antibodies in the protection against or detection of a cellular activity or for the presence of a selected cell surface receptor or the diagnosis of disease. Thus, the subject composition of the present invention may be provided, usually in a lyophilized form in a container, either alone or in conjunction with additional antibodies specific for the desired cell type. The produced antibodies, which may be conjugated to a label or toxin, or unconjugated, are included in the kits with buffers, such as Tris, phosphate, carbonate, etc., stabilizers, biocides, inert proteins, e.g., serum albumin, or the like, and a set of instructions for use. Generally, these materials will be present in less than about 5% wt. based on the amount of active antibody, and usually present in total amount of at least about 0.001% wt. based again on the antibody concentration. Frequently, it will be desirable to include an inert extender or excipient to dilute the active ingredients, where the excipient may be present in from about 1 to 99% wt. of the total composition. Where a second antibody capable of binding to the modified antibody is employed in an assay, this will usually be present in a separate vial. The second antibody is typically conjugated to a label and formulated in an analogous manner with the antibody formulations described above.

All references cited herein, including publications, patents, and patent applications are expressly incorporated by reference in their entireties.

### EXAMPLES

### Example 1

This example describes the generation of chicken anti-IL-12 monoclonal antibodies.

A chicken monoclonal antibody, termed B1, which binds to both human and mouse IL-12 was isolated by phage display in the scFv form. After conversion to whole antibody in the chicken-human IgG1/λ chimeric form, B1 retained the property to bind to human and mouse IL-12.

Materials and Methods

Chicken Immunization

A female White Leghorn chicken was immunized intramuscularly with 100 μg of recombinant human IL-12 in complete Freund adjuvant. The chicken was boosted with 25 μg of mouse IL-12 (R&D, Minneapolis, Minn.) in incomplete Freund adjuvant at day 21 and with 25 μg of human IL-12 (R&D) in incomplete Fruend adjuvant at day 35. The spleen was harvested at day 40. The chicken immunization was performed by BAbCO (Richmond, Calif.).

Plasmids

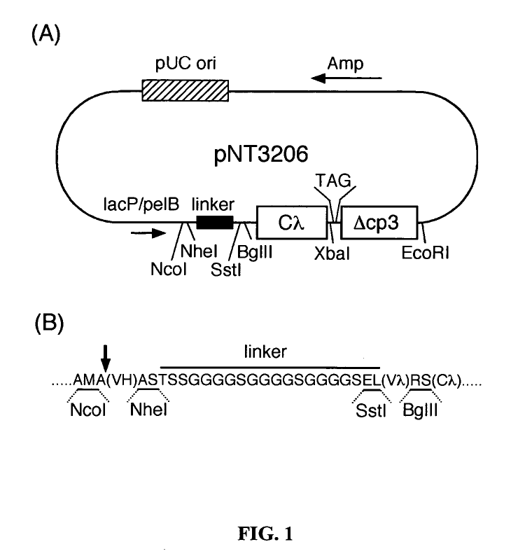

The M13 phage display vector pNT3206 (FIG. 1), a derivative of pScUAGΔcp3 (Akamatsu, Y., et al., J. Immunol. 151: 4651-4659 (1993)), carries the human Cλ gene in place of the human Cκ gene. With pNT3206, an antibody is expressed as a single chain Fv (scFv) fragment fused to human Cλ and secreted to E. coli periplasm.

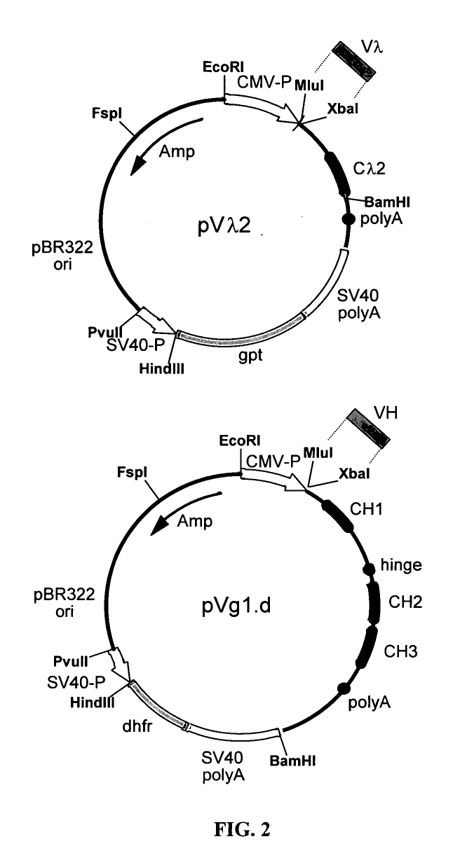

The mammalian expression vector pVg1.d for production of human γ1 heavy chain was described previously (Co, M.S., et al., J. Immunol. 148: 1149-1154 (1992)). The mammalian expression vector pVλ2 for production of human λ2 light chain, a derivative of the human κ light chain expression vector pVk (Co, M.S., et al., J. Immunol. 148: 1149-1154 (1992)), was constructed by first replacing the XbaI-BamHI fragment of pVk containing the genomic human κ constant region with an XbaI-BglII PCR product containing the genomic human λ1 constant region (pVλ1). To make pVλ2, the Cλ1 coding region in pVλ1 was converted to a Cλ2 coding region by site-directed mutagenesis. The schematic structures of pVg1.d and pVλ2 are shown in FIG. 2.

Expression of Recombinant Human IL-12

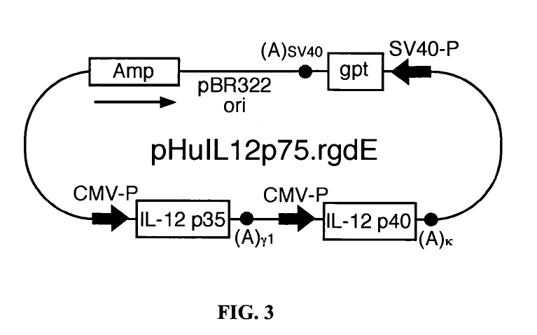

The cDNA's for the entire coding region, including the signal peptide-coding region, of human IL-12 p35 and p40 chains were separately amplified by PCR using human peripheral blood mononuclear cells (PBMC)-derived cDNA as a template. For cloning of human IL-12 p35 subunit, PCR primers MSC12p35-15′-GGGGGCGCCAGCGGCTCGCCCTGTGTC-3′ (SEQ ID NO: 17) and PCR primer MSC12p35-2 5′-CCCGGCGCCGACAACGGTTTGGAGGGACCTC-3′ (SEQ ID NO: 18) were used. For cloning of human IL-12 p40 subunit, PCR primers MSC12p40-15′-GGGTCTAGAGCCATTGGACTCTCCGTCCTG-3′ (SEQ ID NO: 19) and MSC12p40-25′-CCCGCTCAGCCCTCCAAATTTTCATCCTGGATC-3′ (SEQ ID NO: 20) were used. The cDNA's encoding p35 and p40 chains were then cloned into pOKT3.IgG2.rg.Tt (Cole, M. S., et al., J. Immunol. 159: 3613-3621 (1997)) to replace the heavy and light chain coding regions, respectively. The resulting plasmid, pHuIL12p75.rgdE (FIG. 3), was introduced into the chromosome of the mouse myeloma cell line NS0 by electroporation as described (Cole, M. S., et al., J. Immunol. 159: 3613-3621 1997)). A mycophenolic acid-resistant NS0 stable transfectant producing a high level of human IL-12 p70 heterodimer was adapted to and expanded in Hybridoma SFM (Life Technologies, Rockville, Md.). Recombinant human IL-12 p70 heterodimer used for chicken immunization was purified from culture supernatant by two-step column chromatography using first mono Q Sepharose (Pharmacia, Piscataway, N.J.) and then heparin Sepharose (Pharmacia).

Expression of Soluble Human IL-12 Receptor β2 Chain

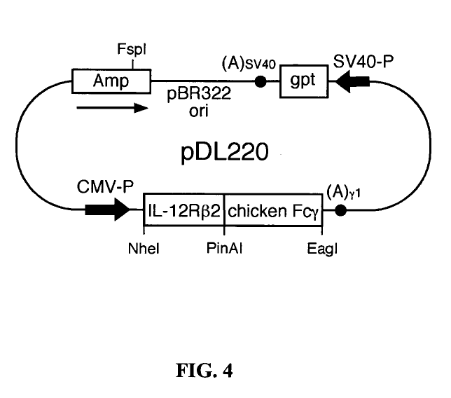

The cDNA for the extracellular region of human IL-12 receptor β2 chain (IL-12Rβ2; amino acids 1 to 599 of mature protein) was amplified by PCR using human PBMC-derived cDNA as a template. The PCR primers HF 190 5′-CTTCGTGCTAGCG TCCACTCCAATATAGATGTGTGCAAGCTTGGC-3′ (SEQ ID NO: 21) and HF 191 5′-CTGAGCCACACCGGTGTTGGCTTTGCCCTGTGG (SEQ ID NO: 22) were used. The PCR-amplified fragments were digested with NheI and PinAI, and cloned into corresponding sites of a mammalian expression vector derived from pOKT3.Vk.rg (Cole, M. S., et al., J. Immunol. 159: 3613-3621 (1997)) to make a fusion of human IL-12Rβ2 extracellular region to the Fc region of chicken immunoglobulin γ heavy chain (amino acid position 210 to 610 according to the Kabat numbering (Johnson, G. and Wu, T. T., Nucleic Acids Res. 28: 214-218 (2000)) with a polypeptide linker Thr-Gly-Gly-Gly. The resulting plasmid, pDL220 (FIG. 4), was linearized with FspI and stably transfected into NS0 cells by electroporation. A mycophenolic acid-resistant NS0 stable transfectant producing a high level of human IL-12Rβ2-chicken Fcγ fusion proteins was adapted to and expanded in Hybridoma SFM (Life Technologies). The fusion protein was purified from culture supernatant by column chromatography using Sepharose coupled with rabbit anti-chicken Ig polyclonal antibodies (Jackson ImmunoResearch, West Grove, Pa.). Construction of chicken scFv library

Total RNA was extracted from chicken spleen cells using TRIzol reagent (Life Technologies) and poly(A)⁺RNA was isolated with the PolyATract MRNA isolation system (Promega, Madison, Wis.) according to the suppliers' protocols. First-strand cDNA was synthesized using poly(A)⁺RNA as a template and random hexadeoxynuleotides as primers. The reaction was performed with SuperScript II reverse transcriptase (Life Technologies) according to the supplier's protocol.

Chicken VH genes were amplified by PCR using a 5′ primer CCAGCACCCATGGCCGCCGTGACGTTGGACGAGTCCG (NT561) (SEQ ID NO: 23) and a 3′ primer CGTCAAGCTAGCGGAGGAGACGATGACTTCGGTCCC (NT563) (SEQ ID NO: 24). The NcoI site in NT561 and the NheI site in NT563 are underlined. Chicken Vλ genes were amplified using a 5′ primer CACGCAGAGCTCGCGCTGACTCAGCCG(TG)CCTC(GA)GT (NT562) (SEQ ID NO: 25) and a 3′ primer AGCCACAGATCTTAGGACGGTCAGGGTTGTCCCG (NT564) (SEQ ID NO: 26). The SstI site in NT562 and the BglII site in NT564 are underlined.

The construction of a scFv library and rescue of phagemid were carried out essentially according to Akamatsu, Y., et al., J. Immunol. 151: 4651-4659 (1993). PCR-amplified fragments were gel-purified and digested with NcoI and NheI (for VH) or SstI and BglII (for Vλ). The digested VH and Vλ fragments were gel-purified and ligated with correspondingly digested pNT3206 to construct VH and Vλ libraries, respectively. Plasmid DNA of these two libraries were then digested with EcoRI and NheI. The VH- and Vλ-containing fragments (˜4.3 kb and ˜1.3 kb, respectively) were ligated to make a combinatorial library and electroporated into E. coli DH5α/F′IQ. Cells were grown in SOC broth (Sambrook, J., et al., Molecular Cloning: A Laboratory Manual, 2nd ed., Cold Spring Harbor Laboratory, NY (1989)) for 60 min at 37° C. and an aliquot was plated on an LB plate containing 50 μg/ml ampicillin to measure the library size. The rest was grown at 37° C. in 2YT broth (Sambrook, J., et al. (1989)) with 50 μg/ml ampicillin and 1% glucose until the OD₆₀₀ reached 1.0, when VCSM13 helper phage were infected. The cells were further grown overnight in 2YT broth with 50 μg/ml ampicillin and 75 μg/ml of kanamycin. Phage particles were purified and concentrated from culture supernatant by two rounds of precipitations with polyethylene glycol (PEG) (Sambrook, J., et al., (1989)), and resuspended in 10 ml of 25 mM HEPES-NaOH (pH 7), 150 mM NaCl (HBS). The phagemid titer, measured as ampicillin-resistant colony-forming unit (cfu), was 10¹² per ml.

Selection of Anti-IL-12 scFv Antibodies

Phage (5×10¹² cfu) in HBS containing 0.1% BSA (HBS-BSA) were first loaded on an anti-human λ chain affinity column prepared by coupling goat polyclonal anti-human λ light chain antibodies (BioSource, Camarillo, Calif.) to CNBr-activated Sepharose 4B (Sigma, St. Louis, Mo.) to enrich phage particles displaying scFv-human Cλ fusion proteins on the surface. After washing the column with HBS, phage were eluted with 0.2 M glycine-HCl (pH 2.1), neutralized with 2 M Tris Base, and mixed with the equal volume of HBS-BSA. Eluted phages were loaded on a casein agarose column to eliminate non-specific binders. The flow-through fraction was used for subsequent binding to IL-12. For each cycle of selection, phages were treated with an anti-human λ chain column and a casein agarose column as described above.

Phages were then incubated in several wells of a 96-well ELISA plate (MaxiSorp, Nunc Nalge, Naperville, Ill.) coated with 1 μg/ml of human IL-12 at room temperature for 2 hrs. After washing wells with HBS, bound phage were eluted with 0.2 M glycine-HCl (pH 2.1) and neutralized with 2 M Tris Base for the first two rounds of selection. For the third and fourth rounds of selection, bound phage were competitively eluted by incubating with 15 μg/ml of recombinant soluble human IL-12 receptor β2 chain at room temperature for 1 hr. For the first three rounds, eluted phages were used to infect logarithmically growing TG1ΔrecA cells (Akamatsu, Y., et al., J. Immunol. 151: 4651-4659 (1993)). Rescue of phagemid by VCSM13 superinfection was carried out as described in the previous section. PEG-concentrated phage were used for the next round of selection.

Soluble scFv-Cλ fusion proteins were produced by growing ampicillin-resistant TG1ΔrecA transformants in 2YT broth with 1% glycerol and 1 mM IPTG at 30° C. overnight. Culture supernatants were used for ELISA to detect binding of scFv-Cλ fusion proteins to IL-12. MaxiSorp plates were coated with 0.2 μg/ml of human or mouse IL-12 (R&D) in 0.2 M sodium carbonate buffer (pH 9.4) and blocked with SuperBlock buffer (Pierce, Rockford, Ill.). Detection of bound scFv-Cλ was carried out by incubating with HRP-conjugated goat anti-human λ chain antibodies (Southern Biotechnology Associates, Birmingham, Ala.). Color development was performed with TMB substrate (Kirkegaard & Perry Laboratories, Gaithersburg, Md.) as described by supplier. Absorbance was read at 450 nm using an VERSAmax microplate reader (Molecular Devices, Menlo Park, Calif.).

Results

Isolation of Chicken Anti-IL-12 scFv Antibodies

A female white Leghorn chicken was intramuscularly immunized with 100 μg of human IL-12 on day 0, boosted by injecting 25 μg of mouse IL-12 on day 21, and further boosted by injecting 25 μg of human IL-12 on day 35. ELISA analysis of chicken sera collected right before the first injection (pre-bleed), on day 26 (after two injections) and on day 40 (after three injections) showed a strong immune response to IL-12 after two injections. The third injection only slightly increased the serum titers to human and mouse IL-12.

The spleen of the immunized chicken was harvested on day 40 and total RNA was immediately isolated. A phage display library for expression of chicken scFv antibody was constructed as described in Materials and Methods. The library sizes were 10⁷ for the VH library, 10⁷ for the Vλ library, and 3.5×10⁷ for the VH-Vλ combinatorial library. The phage particles of the combinatorial library, which was obtained by infecting DH5α/F′IQ transformants with VCSM13 helper phage, was used for isolation of chicken scFv antibodies that bind to human IL-12. For the first two rounds of selection, phages were selected by binding to human IL-12 coated in an ELISA plate and elution by 0.2 M glycine-HCl (pH 2.1). At the end of each round, the eluted phages were propagated by infecting DH5α/F′IQ followed by superinfection of VCSM13 helper phage. At the third and fourth rounds, phage bound to human IL-12 were competitively eluted by incubation with soluble human IL-12 receptor β2 chain fused to chicken Fcγ.

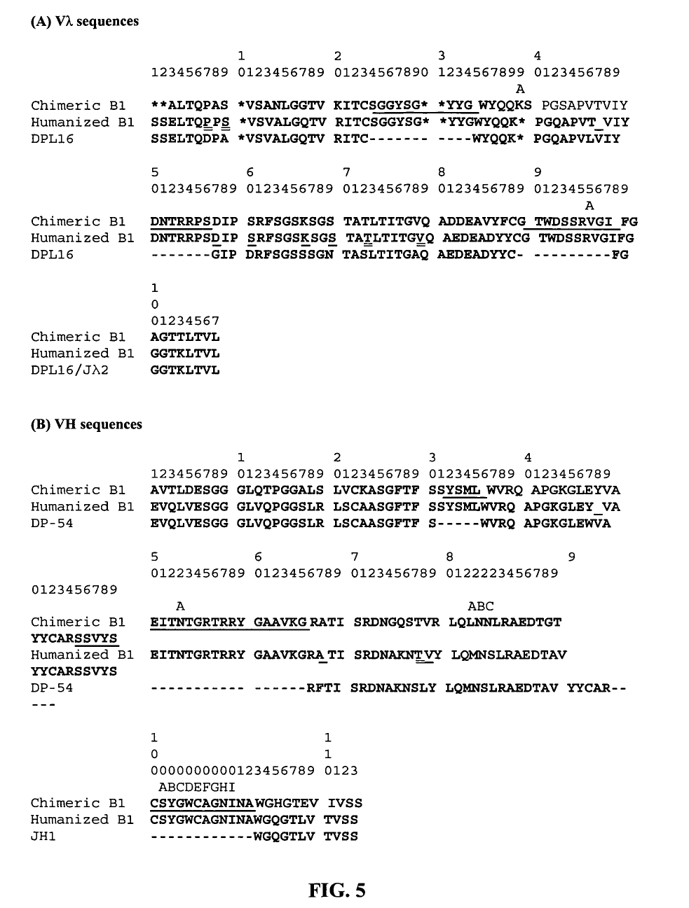

At the end of the fourth round of selection, selected phage were used to infect E. coli TG1ΔrecA. Ampicillin-resistant TG1ΔrecA transformants were individually cultured and expression of soluble scFv was induced by IPTG. The culture supernatants were used for ELISA to identify the clones which bind to both human and mouse IL-12. Among many clones which showed specific binding to human IL-12, several clones were found to also bind to mouse IL-12. Among them, the clone B1 showed strong binding to both human and mouse IL-12. The amino acid sequences of mature VH (SEQ ID NO: 2) and Vλ (SEQ ID NO: 4) of B1 are shown in FIG. 5.

### Example 2

This example describes the characterization of B1 in the chimeric IgG1/λ form.

Materials and Methods

Conversion of scFv to Whole Antibody

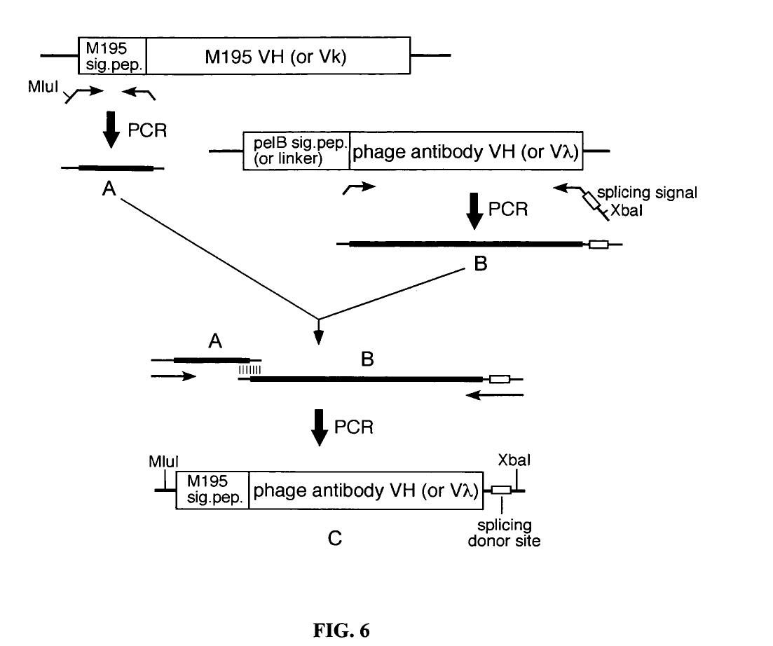

The VH and Vλ genes of chicken anti-IL-12 scFv antibodies obtained by phage display were converted to an exon for cloning into pVg1.d and pVλ2, respectively. The signal peptide-coding regions of mouse anti-CD33 M195 VH and Vk (Co, M.S., et al., J. Immunol. 148: 1149-1154 (1992)) were connected to the coding region of VH and Vλ of chicken anti-IL-12 scFv, respectively, by the recombinant PCR method (Higuchi, R., PCR Technology: Principles and Applications for DNA Amplification, Stockton Press, NY, pp. 61-70 (1989). A splicing donor site was attached to the 3′ end of the coding region of the VH and Vλ genes. In addition, MluI and XbaI sites were placed at the 5′ and 3′ ends of the V genes, respectively. The scheme for the construction of V exons is shown in FIG. 6.

Results

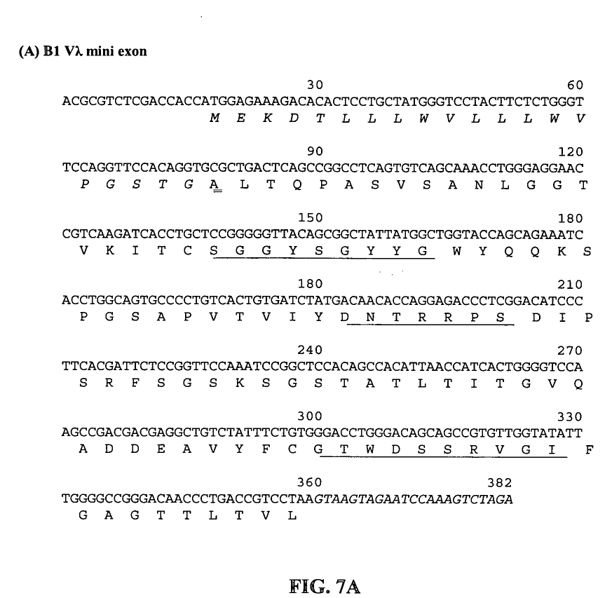

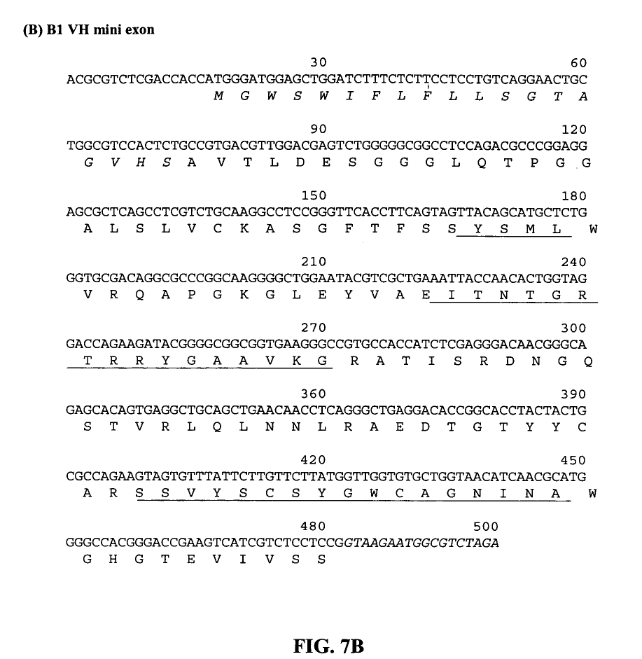

For further analysis, the chicken scFv anti-IL-12 antibody B1 was converted to whole antibody in the form of chicken-human chimeric IgG1/λ. Each of the B1 Vλ and VH genes was changed to a mini-exon, including a signal peptide-coding region and a splicing donor site as outlined in FIG. 6. The nucleotide sequences (SEQ ID Nos: 47 and 49) and deduced amino acid sequences (SEQ ID Nos: 48 and 50) of the B1 Vλ and VH mini-exons are shown in FIGS. 7A and 7B, respectively. The B1 Vλ and VH mini-exons were cloned into mammalian expression vectors pVλ2 and pVg1.d, respectively (FIG. 2). The resulting plasmids, pVλ2-B1 and pVg1-B1, were cotransfected into a mouse myeloma cell line Sp2/0 by electroporation. Sp2/0 stable transfectants were selected in the mycophenolic acid medium and screened for production of chimeric B1 IgG1/λ by ELISA as described in Materials and Methods. One of the high producing Sp2/0 transfectants, clone #32, was adapted to growth in serum-free medium and expanded for purification of chimeric B1 IgG1/λ antibodies with a protein A affinity column. Purified chimeric B1 showed specific binding to human and mouse IL-12 (data shown later), showing that chimeric B1 retains the binding specificity of the parental chicken anti-IL-12 scFv antibody.

### Example 3

This example describes the humanization of chicken anti-IL-12 antibodies. Humanization of the chicken monoclonal antibody B1 was carried out according to the present invention. First, a human V segment with a high homology to the B1 VH or Vλ amino acid sequence was identified. Next, the chicken CDR sequences together with chicken framework amino acids important for maintaining the CDR structure were grafted into the selected human framework sequence. In addition, human framework amino acids which were found to be rare in the corresponding V subgroup were substituted by the consensus amino acids to reduce potential immunogenicity. The resulting humanized B1 IgG1/λ monoclonal antibody was expressed in the mouse myeloma cell line Sp2/0. By the competitive binding assay using purified humanized and chimeric B1 antibodies, the affinities of humanized B1 to human and mouse IL-12 were shown to be approximately 1.4 fold and 2.0 fold better than that of chimeric B1.

Materials and Methods

Humanization

Humanization of the chicken antibody V regions was carried out as outlined in the present invention. The human V region framework used as an acceptor for the CDR's of the chicken anti-IL-12 monoclonal antibody B1 was chosen based on sequence homology. The computer programs ABMOD (Zilber, B., Scherf, T., Levitt, M., and Anglister, J. Biochemistry 29:10032-41 (1990)) and ENCAD (Levitt, M., J. Mol. Biol. 168: 595-620 (1983)) were used to construct a molecular model of the variable regions. Amino acids in the humanized V regions predicted to have contact with CDRs were substituted with the corresponding residues of chicken B1. The amino acids in the humanized V region that were found to be rare in the same V region subgroup were changed to the consensus amino acids to eliminate potential immunogenicity.

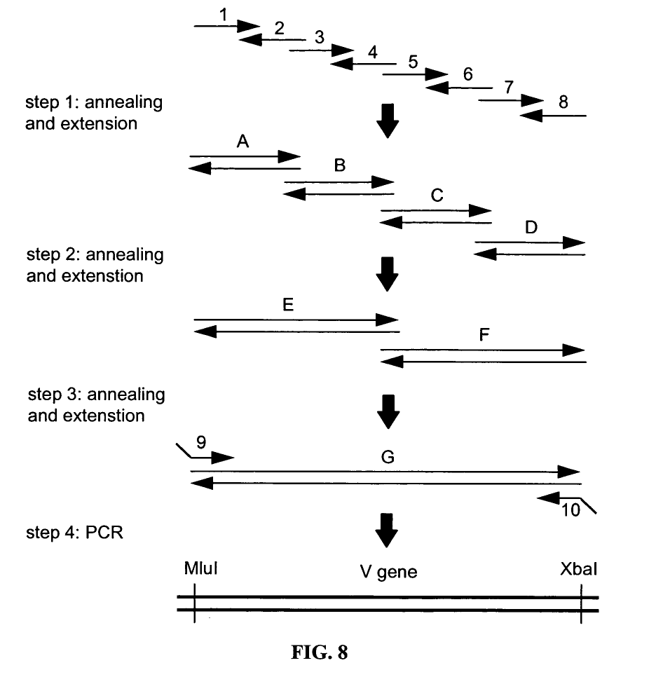

The light and heavy chain variable region genes were constructed and amplified using eight overlapping synthetic oligonucleotides ranging in length from approximately 65 to 80 bases as illustrated in FIG. 8 (He, X., et al., J. Immunol. 160: 1029-1035 (1998)). The oligonucleotides were annealed pairwise and extended with the Klenow fragment of DNA polymerase I, yielding four double-stranded fragments. The resulting fragments were denatured, annealed pairwise, and extended with Klenow, yielding two fragments. These fragments were denatured, annealed pairwise, and extended once again, yielding a full-length gene. The resulting product was amplified by PCR using the Expand High Fidelity PCR System (Roche Molecular Biochemicals, Indianapolis, Ind.). The PCR-amplified fragments were gel-purified and cloned into pCR4Blunt-TOPO vector. After sequence confirmation, the VL and VH genes were digested with MluI and XbaI, gel-purified, and subcloned respectively into pVλ2 and pVg1.d for expression of light and heavy chains to make pVλ2-HuB1 and pVg1-HuB1, respectively.

Stable Transfection

Mouse myeloma cell line Sp2/0-Ag14 (referred to as Sp2/0 in this text) was obtained from ATCC (Manassas, Va.) and maintained in DME medium containing 10% FBS (HyClone, Logan, Utah) at 37° C. in a 7.5% CO₂ incubator.

Stable transfection into Sp2/0 was carried out by electroporation as described in Co, M. S., et al., J. Immunol. 148: 1149-1154 (1992). Before transfection, the light and heavy chain expression vectors were linearized using FspI. The transfected cells were suspended in DME medium containing 10% FBS and plated into several 96-well plates. After 48 hr, selection media (DME medium containing 10% FBS, HT media supplement, 0.3 mg/ml xanthine and 1 μg/ml mycophenolic acid) was applied. Approximately 10 days after the initiation of selection, culture supernatants were assayed for antibody production by ELISA. High-yielding Sp2/0 transfectants were expanded in DME medium containing 10% FBS and then adapted to growth in serum-free medium using Hybridoma SFM (Life Technologies).

Expression of chimeric B1 (ChB1) and humanized B1 (HuB1) antibodies was measured by sandwich ELISA. MaxiSorp plates (Nunc Nalge) were coated with 1 μg/ml goat anti-human y chain polyclonal antibodies (Jackson) and blocked with Superblock Blocking Buffer (Pierce). Samples containing ChB1 or HuB1 were appropriately diluted in ELISA buffer (PBS containing 1% BSA and 0.1% Tween 20) and applied to ELISA plates. As a standard, human IgG1/λ2 antibody OST-577 (anti-HBV; Ehrlich, P. H., et al., Hum. Antibodies Hybridomas 3: 2-7 (1992)) was used. Bound antibodies were detected by HRP-conjugated goat anti-human λ chain polyclonal antibodies (Southern Biotechnology). Color development was performed with ABTS substrate. Absorbance was read at 415 nm using a VERSAmax microplate reader (Molecular Devices, Menlo Park, Calif.).

Purification of Anti-IL-12 Antibodies

Sp2/0 stable transfectants were grown to exhaustion in Hybridoma SFM. After centrifugation and filtration, culture supernatant was loaded onto a protein-A Sepharose column. The column was washed with PBS before the antibody was eluted with 0.1 M glycine-HCl (pH 2.8), 0.1 M NaCl. After neutralization with 1 M TrisHCl (pH 8), the eluted protein was dialyzed against PBS and stored at 4° C. Antibody concentration was determined by measuring absorbance at 280 nm (1 mg/ml =1.4 A₂₈₀). SDS-PAGE in Tris-glycine buffer was performed according to standard procedures.

ELISA

For titration experiments, ELISA plates were coated with 0.1 μg/ml of human or mouse IL-12 (R&D) in 0.2 M sodium carbonate buffer (pH 9.4) and blocked with SuperBlock buffer (Pierce). Humanized or chimeric B1 antibody was added to wells in triplicate (starting at 1.11 μg/ml and serial 3-fold dilutions). After incubating at room temperature for 2 hr, bound chimeric or humanized B1 antibodies were detected by incubating with HRP-conjugated goat anti-human Fcγ antibodies (Jackson ImmunoResearch). Color development was performed with TMB substrate (Kirkegaard & Perry Laboratories).

For binding to various proteins, ELISA plates were coated with chicken lysozyme (Sigma), human globin (Sigma), bovine albumin (Sigma), and concanavalin A (Pharmacia) as well as human and mouse IL-12 (R&D) at 0.1 μg/ml in 0.2 M sodium carbonate buffer (pH 9.4) and blocked with SuperBlock buffer (Pierce). Humanized or chimeric B1 antibody was added at 0.01 μg/ml in ELISA buffer. After incubating at room temperature for 2 hr, bound chimeric or humanized B1 antibodies were detected by incubating with HRP-conjugated goat anti-human Fcλ antibodies followed by incubation with TMB substrate.

Competition ELISA

MaxiSorp plates were coated with 100 μl of 0.1 μg/ml human or mouse IL-12 (R&D) and blocked with Superblock blocking buffer. A mixture of biotinylated humanized B1 (0.5 μg/ml final concentration) and competitor antibody (chimeric or humanized B1 starting at 200 μg/ml final concentration and serial 3-fold dilutions) in 100 μl ELISA buffer were added in triplicate. As a background control, 100 μl of ELISA Buffer was used. ELISA plates were incubated at room temperature for 2 hr. After washing the wells with Washing Buffer (PBS containing 0.1 % Tween 20), 100 μl of 1 μg/ml HRP-conjugated streptavidin (Pierce) in ELISA buffer was added to each well. ELISA plates were incubated at room temperature for 30 min and washed with Washing Buffer. For color development, 100 μI/well of ABTS substrate was added. Color development was stopped by adding 100 μl/well of 2% oxalic acid. Absorbance was read at 415 nm.

Results

Humanization of Chicken Antibodies

For humanization of the chicken B1 variable regions, the general approach provided in the present invention was followed. First, a molecular model of the B1 variable regions was constructed with the aid of the computer programs ABMOD (Zilber, B., Scherf, T., Levitt, M., and Anglister, J. Biochemistry 29:10032-41 (1990)) and ENCAD (Levitt, M., J. Mol. Biol. 168: 595-620 (1983)). Next, based on a homology search against human germline V and J segment sequences, the Vλ segment DPL16 (Williams, S. C. and Winter, G., Eur. J. Immunol. 23: 1456-1461 (1993)) and the J segment Jλ2 (Udey, J. A. and Blomberg, B., Immunogenetics 25: 63-70 (1987)) were selected to provide the frameworks for the B1 light chain variable region. For the B1 heavy chain variable region, the VH segment DP-54 (Tomlinson, I. M., et al., J. Mol. Biol. 227: 776-789 (1992)) and the J segment JH1 (Ravetch, J. V., et al., Cell 27: 583-591 (1981)) were used. The identify of the framework amino acids between chicken B1 Vλ and the acceptor human DPL16 and Jλ2 segments was 70%, while the identity between chicken B1 VH and the human DP-54 and JH1 segments was 72%.

At framework positions in which the computer model suggested significant contact with the CDRs, the amino acids from the chicken V regions were substituted for the original human framework amino acids. This was performed at residues 46, 57, 60, 66 and 69 of the light chain (FIG. 5A). For the heavy chain, replacements were made at residues 47, 67, and 78 (FIG. 5B). In addition, human framework residues that were found to be rare in the same V region subgroup were changed to the corresponding consensus amino acids to eliminate potential immunogenicity. For humanized B1, this was performed at residues 7, 9, 72 and 78 in the light chain (FIG. 5A) and at residue 77 in the heavy chain (FIG. 5B). The alignment of the amino acid sequences of the chicken B1, humanized B1, and human germline V and J segments for light and heavy chain variable regions are shown in FIGS. 5A and 5B, respectively.

It should be noted that chicken Vλ sequences contain two amino acid deletions and one amino acid insertion compared to human Vλ sequences. The N-terminal amino acid of chicken Vλ corresponds to the third amino acid from the N-terminus of human Vλ (FIG. 5A). The framework 2 of chicken Vλ contains an extra amino acid (residue at 39A in FIG. 5A). The molecular model of the B1 variable regions suggested that the addition of two amino acids at the N-terminus of B1 Vλ would not change the CDR structure. The model also predicted that a serine residue at position 39A exists in the loop located opposite and away from the antigen binding site and is unlikely to interact with the CDR nor introduce a significant structural change in the framework when it is deleted. Therefore, in the humanized B1 Vλ sequence, the two N-terminal amino acids of the acceptor human DPL16 segment were added and a serine residue at position 39A in the chicken B1 Vλ sequence was eliminated.

Expression of Humanized B1 IgG1/λ Antibody

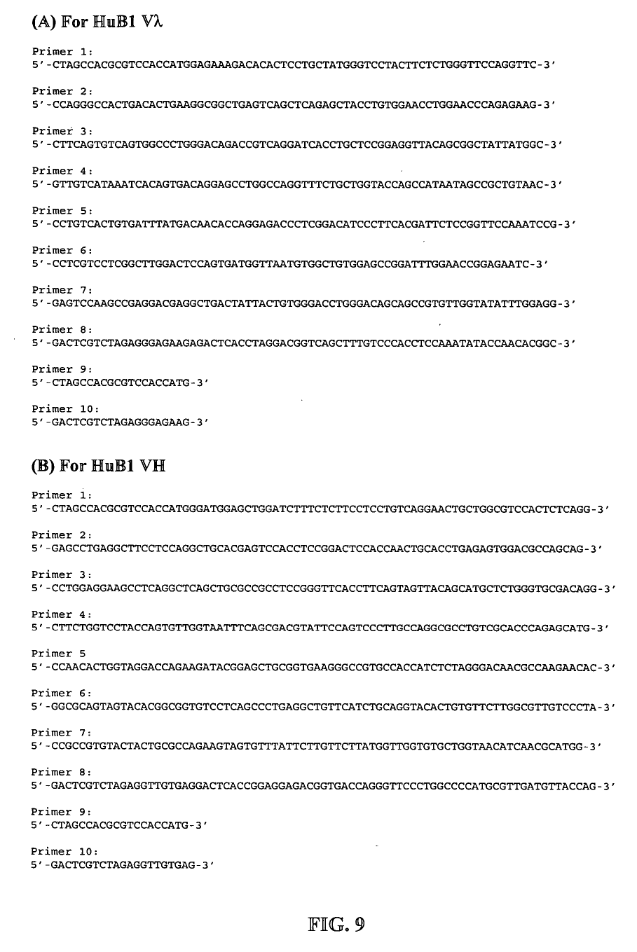

A gene encoding humanized Vλ or VH was designed as a mini-exon including a signal peptide, a splice donor signal, and appropriate restriction enzyme sites for subsequent cloning into a mammalian expression vector. The splicing donor signal and the signal peptide sequence in each of the humanized Vλ and VH mini-exons were derived from the corresponding chimeric B1 mini-exon. The humanized B1 Vλ and VH genes were constructed by extension of eight overlapping synthetic oligonucleotides and PCR amplification as illustrated in FIG. 8. A series of 8 overlapping oligonucleotides (1˜8) were used. Oligonucleotides land 2, 3 and 4, 5 and 6, and 7 and 8 were separately annealed and extended with the Klenow fragment of DNA polymerase I. The resulting double-stranded DNA fragments, A and B, and C and D, were separately mixed, denatured, annealed and extended to yield the DNA fragments E and F, respectively, which were then mixed to generate the entire mini-exon (G) in the third annealing-and-extension step. The mini-exon was amplified by PCR with primers 9 and 10. The resulting fragments carry the flanking MluI and XbaI sites. Primers 1-10 for the synthesis of humanized heavy chain variable region are shown in FIG. 9A (SEQ ID NO: 27-36, respectively). Primers 1-10 for the synthesis of humanized light chain variable region are shown in FIG. 9B (SEQ ID NO: 37-46, respectively).

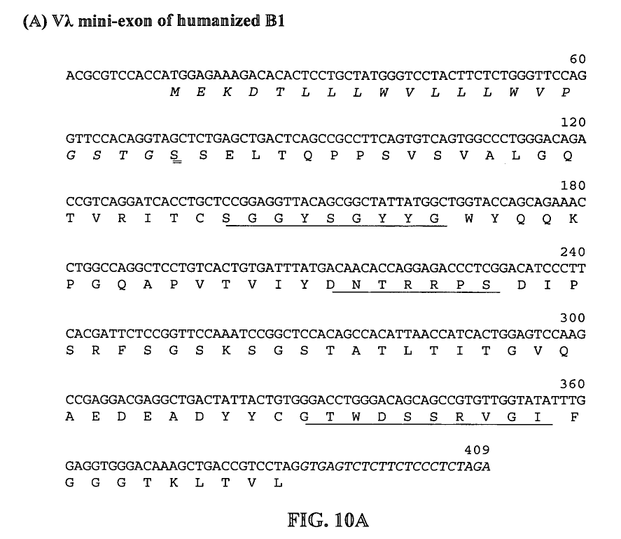

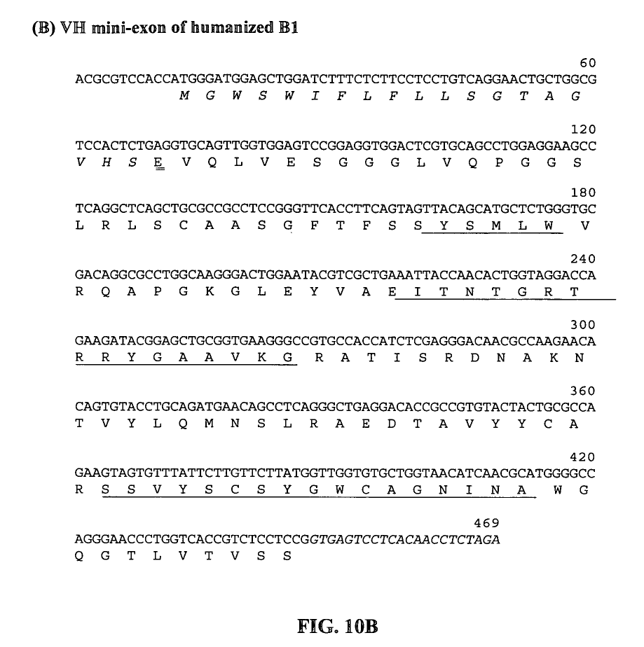

The resulting V gene fragments were cloned into pCR4Blunt-TOPO vector. After sequence confirmation, humanized B1 Vλ and VH genes were digested with MluI and XbaI, and subcloned into mammalian expression vectors, pVλ2 and pVg1.d (FIG. 2), for expression of light and heavy chains, respectively. The DNA sequences (SEQ ID Nos: 51 and 53) and deduced amino acid sequences (SEQ ID Nos: 52 and 54) of the humanized Vλ and VH mini-exons are shown in FIG. 10A and 10B, respectively.

To obtain cell lines stably producing HuB1, pVλ2-HuB1 and pVg1-HuB1 were introduced into the chromosome of mouse myeloma cell line Sp2/0 by electroporation. Stable transfectants were selected for gpt expression as described in Materials and Methods. Culture supernatants of Sp2/0 stable transfectants were analyzed by ELISA for production of HuB1. One of the high producing cell lines, clone H7, was adapted to and expanded in Hybridoma SFM. Humanized B1 IgG1/λ monoclonal antibody was purified from spent culture supernatant with a protein-A Sepharose column as described in Materials and Methods. SDS-PAGE analysis under non-reducing conditions indicated that both humanized chimeric B1 have a molecular weight of about 150-160 kD. Analysis under reducing conditions indicated that humanized and chimeric B1 are comprised of a heavy chain with a molecular weight of about 50 kD and a light chain with a molecular weight of about 25 kD. The purity of the antibodies appeared to be more than 95%.

Binding Property of Humanized B1

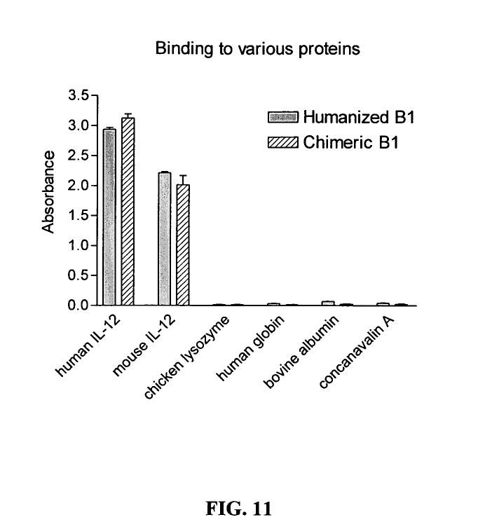

The specificity of the binding of humanized and chimeric B1 was analyzed by ELISA. As shown in FIG. 11, both humanized and chimeric B1 showed good binding to human and mouse IL-12, but not to other proteins (chicken lysozyme, human globin, bovine albumin, and concanavalin A) tested here. This result indicates that the binding specificity of chimeric B1 was not altered during the process of humanization.

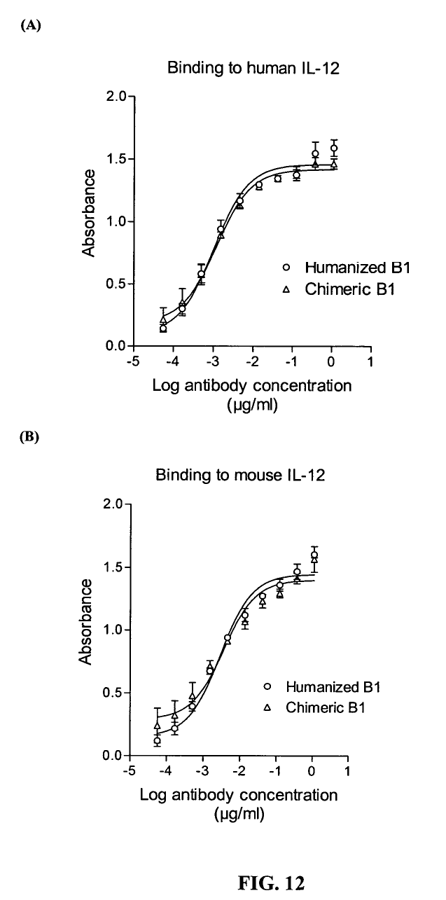

The binding of humanized and chimeric B1 to human and mouse IL-12 was further characterized by ELISA. FIG. 12A shows the titration curves of humanized and chimeric B1 in binding to human IL-12. The titration curves for humanized and chimeric antibodies were almost overlapping to each other. Similarly, the titration curves for humanized and chimeric antibodies in binding to mouse IL-12 were also almost overlapping to each other (FIG. 12B). These results imply that the affinities of chimeric B1 to human and mouse IL-12 are retained in humanized B1.

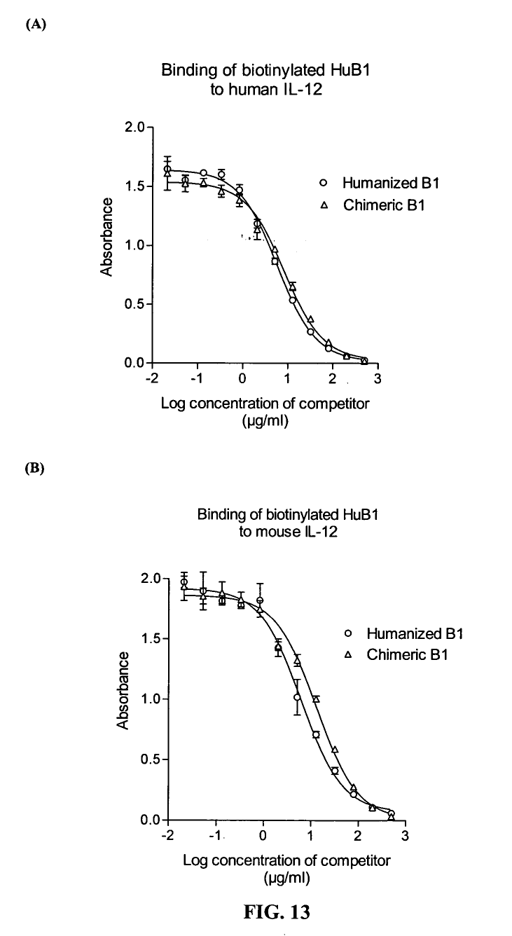

The affinities of humanized and chimeric B1 antibodies to human IL-12 were analyzed by competition ELISA as described in Materials and Methods. A representative result is shown in FIG. 13A. Both humanized and chimeric B1 competed with biotinylated humanized B1 in a concentration-dependent manner. The IC₅₀ values of humanized and chimeric B1, obtained using the computer software Prism (GraphPad Software Inc., San Diego, Calif.), were 5.8 μg/ml and 8.3 μg/ml, respectively. The binding of humanized B1 to human IL-12 was approximately 1.4 fold better than that of chimeric B1. Similarly, the affinities of humanized and chimeric B1 to mouse IL-12 were analyzed by competition ELISA (FIG. 13B). The IC₅₀ value of humanized and chimeric B1 in binding to mouse IL-12 were 6.2 μg/ml and 13.0 μg/ml, respectively. The relative binding of humanized B1 to mouse IL-12 was approximately 2.1 fold less than that of chimeric B1. These results clearly indicate that humanization of chicken anti-IL-12 monoclonal antibody was successful; humanized B1 retained the affinities to human and mouse IL-12.

### Example 4

This example describes the humanization of chicken anti-IL-12 antibodies.

Isolation of Chicken Anti-IL12 Monoclonal Antibodies

Immunization of a female White Leghorn chicken with human and mouse IL-12 was carried out as described in Example 1. The spleen of the immunized chicken was harvested at day 40. Construction of a chicken scFv library with the phage display vector pNT3206 was carried out as described in Example 1. Isolation of phage antibodies that bind to human IL-12 was carried out by panning as described Akamatsu, Y., et al. (J. Immunol. 151: 4651-4659 (1993)). After three cycles of binding to and elution from human IL-12 coated microtiter plates, infection of TG1ΔrecA, and rescue by superinfection of VCSM13 helper phage, several phage clones were found to produce scFv antibodies that bind specifically to human IL-12. One of the clones, DD2, bound to human and mouse IL-12.

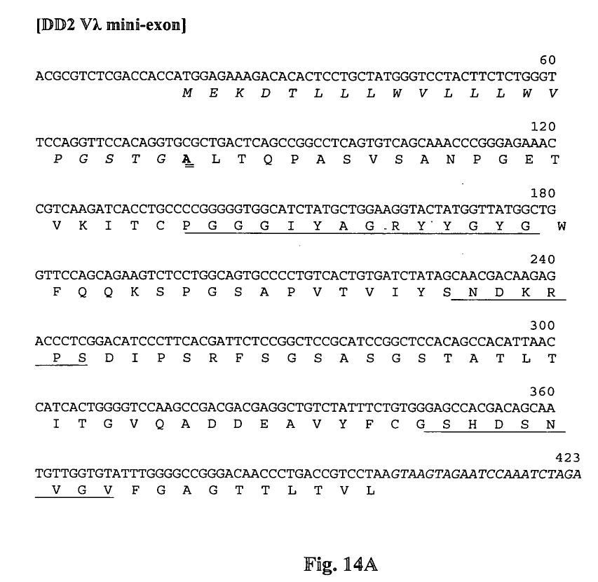

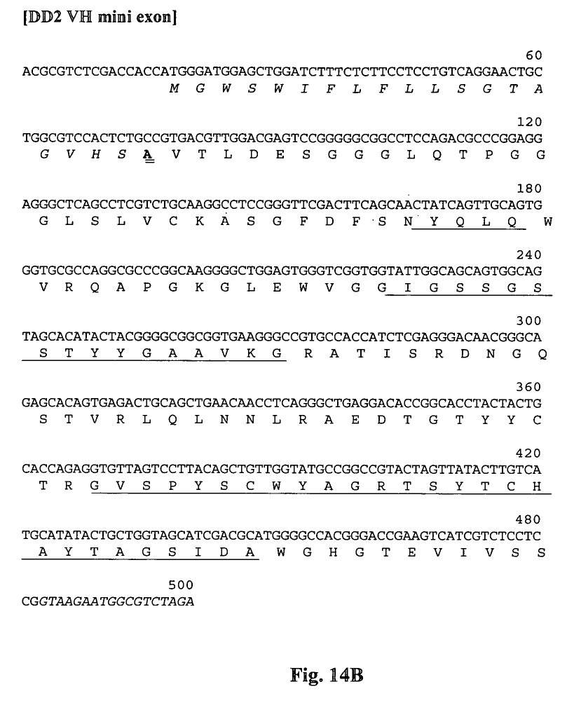

To express DD2 in the form of whole antibody, each of the Vλ and VH genes in the phage display vector was converted to an exon structure containing a signal peptide and a splicing donor site as described in Example 2 (also outlined in FIG. 6). After disgestion with MluI and XbaI, the Vλ and VH genes were cloned into the corresponding sites of the mammalian expression vectors pVλ2 and pVg1.d (FIG. 2), respectively. The nucleotide and deduced amino acid sequences of the DD2 Vλ and VH mini exons are shown in FIG. 14. The resultant vectors were named pVλ2-DD2 for light chain expression and pVg1.d-DD2 for heavy chain expression.

Design of Humanized DD2 Variable Regions

For humanization of the chicken DD2 variable regions, the general approach provided in the present invention was followed. First, a molecular model of the DD2 variable regions was constructed with the aid of the computer programs ABMOD (Zilber, B., Scherf, T., Levitt, M., and Anglister, J. Biochemistry 29:10032-41 (1990)) and ENCAD (Levitt, M., J. Mol. Biol. 168: 595-620 (1983)). Next, based on a homology search against human germline V and J segment sequences, the Vλ segment DPL16 (Williams, S. C. and Winter, G., Eur. J. Immunol. 23: 1456-1461 (1993)) and the J segment Jλ2 (Udey, J. A. and Blomberg, B., Immunogenetics 25: 63-70 (1987)) were selected to provide the frameworks for the DD2 light chain variable region. For the DD2 heavy chain variable region, the VH segment DP-54 (Tomlinson, I. M., et al., J. Mol. Biol. 227: 776-789 (1992)) and the J segment JH1 (Ravetch, J. V., et al., Cell 27: 583-591 (1981)) were used. The identify of the framework amino acids between chicken DD2 Vλ region and the acceptor human DPL16 and Jλ2 segments was 68%, while the identity between chicken DD2 VH and the human DP-54 and JH1 segments was 71%.

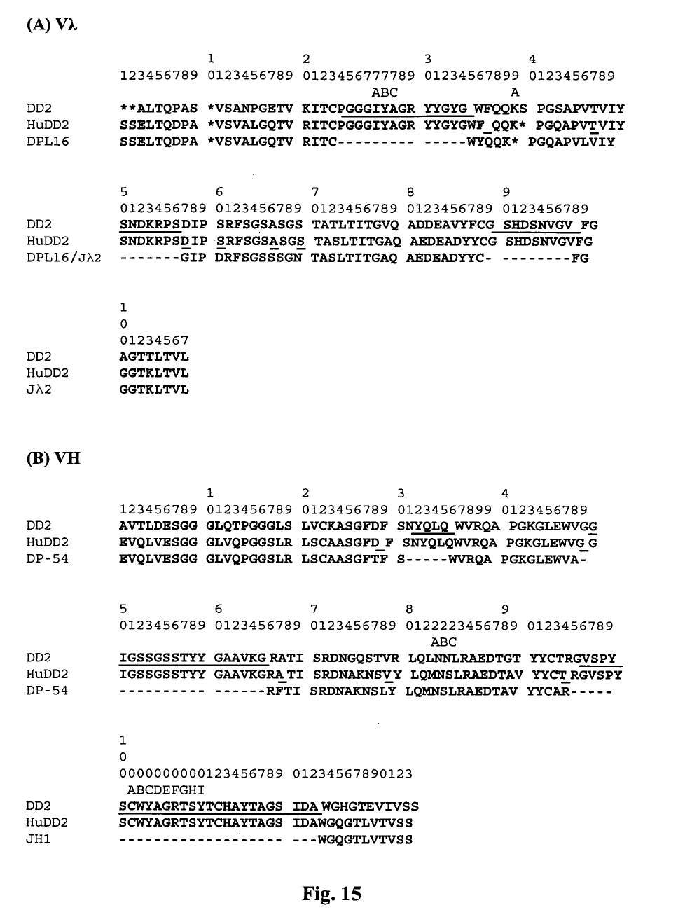

Chicken Vλ regions contain two amino acid deletions and one amino acid insertion compared to human Vλ sequences (FIG. 15). The N-terminal amino acid of chicken mature Vλ corresponds to the third amino acid from the N-terminus of human Vλ. The framework 2 of chicken Vλ contains an extra amino acid at position 39A. Although the first two amino acids at the N-terminus of human mature Vλ exists in the close proximity of the CDR, detailed examination of the model suggests that the transfer of the two serine residues from the DPL 16 Vλ segment to the N-terminus of DD2 Vλ would not drastically change the CDR structure. A serine residue at position 39A in the chicken DD2 Vλ is located in the loop opposite and away from the antigen binding site. The model predicted that the removal of a serine at position 39A during humanization would not introduce a significant change in the CDR or framework structure. Therefore, in the humanized DD2 Vλ sequence, the two N-terminal amino acids of the acceptor human DPL16 segment were added and a serine residue at position 39A in the chicken DD2 Vλ was eliminated.

At framework positions in which the computer model suggested significant contact with the CDRs, the amino acids from the chicken V regions were substituted for the original human framework amino acids. This was performed at residues 36, 46, 57, 60, 66 and 69 of the light chain (FIG. 15). For the heavy chain, replacements were made at residues 28, 49, 67, 78 and 93 (FIG. 15). The alignment of the amino acid sequences of the chicken DD2, humanized DD2 and human acceptor germline V and J are shown segments for both light and heavy variable regions in FIG. 15.

Expression of Humanized DD2 IgG1/λ Antibody

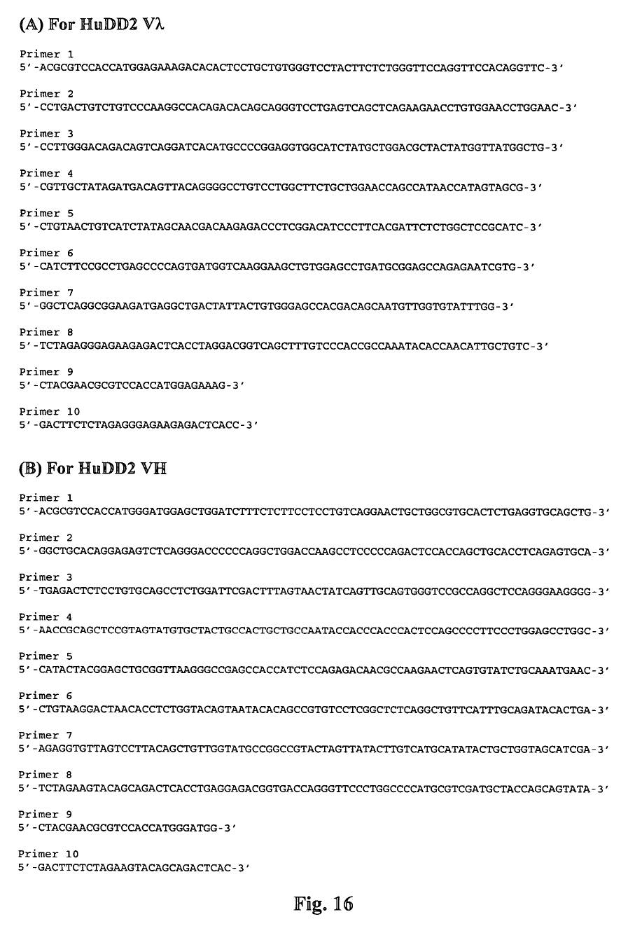

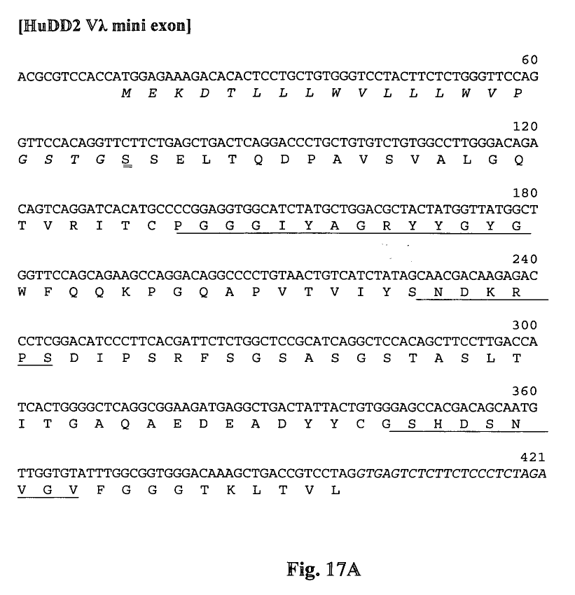

The humanized DD2 Vλ and VH genes, each designed as a mini-exon including a signal peptide, a splice donor signal, and appropriate restriction enzyme sites, were constructed by extension of eight overlapping synthetic oligonucleotides and PCR amplification as described in Example 1 (also outlined in FIG. 6). Primers 1-10 for the synthesis of humanized heavy chain variable region are shown in FIG. 16A (SEQ ID NO: 51-60, respectively). Primers 1-10 for the synthesis of humanized light chain variable region are shown in FIG. 16B (SEQ ID NO: 61-70, respectively).

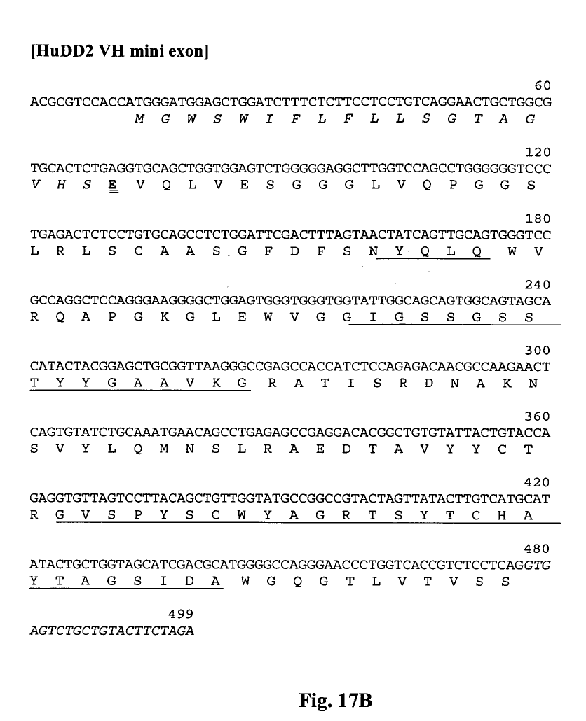

The resulting V gene fragments were digested with MluI and XbaI, and subcloned into mammalian expression vectors, pVλ2 and pVg1.d (FIG. 2), respectively. The resultant plasmids were designated pVλ2-HuDD2 and pVg1.d-HuDD2. The nucleotide sequence and deduced amino acid sequence of the light and heavy chain variable regions of humanized anti-IL-12 antibody DD2 are shown in FIG. 17.

To obtain cell lines stably producing humanized DD2 IgG1/λ monoclonal antibodies (HuDD2), mouse myeloma cell line Sp2/0 was cotransfected with pVλ2-HuDD2 and pVg1.d-HuDD2 after linearization with FspI as described in Example 3. Similarly, Sp2/0 stable transfectants producing ChDD2 were obtained using pVλ2-ChDD2 and pVg1.d-ChDD2. One of the high producing cell lines for each of HuDD2 and ChDD2 was adapted to growth and expanded in serum-free medium (Hybridoma SFM, Invitrogen). HuDD2 and ChDD2 were purified from spent culture supernatant with a protein-A Sepharose column. SDS-PAGE analysis indicated that the purity of each of HuDD2 and ChDD2 was more than 95%.

Binding Properties of Humanized DD2

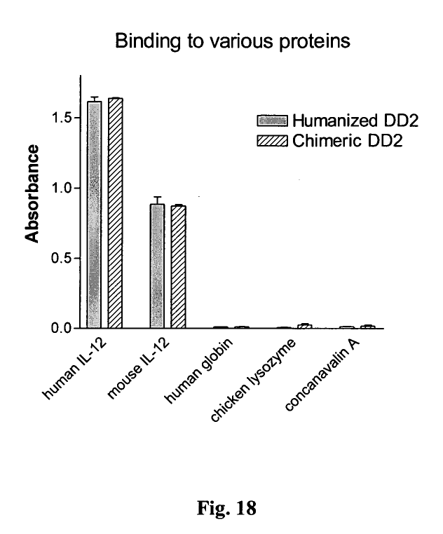

Binding of ChDD2 and HuDD2 to human and mouse IL-12 was examined by ELISA. FIG. 18 shows that HuDD2 at 1 μg/ml bound to human IL-12 as well as ChDD2 did. In addition, HuDD2 bound to mouse IL-12 at the same level as ChDD2. Similar results were obtained at different antibody concentrations in binding to human and mouse IL-12. As shown in FIG. 18, both HuDD2 and ChDD2 at 1 μg/ml exhibited little binding to three control antigens (human globin, hen egg lysozyme, and concanavalin A), indicating that the binding specificity of ChDD2 was retained in HuDD2.

The affinities of ChDD2 and HuDD2 to human and mouse IL-12 were compared by competition ELISA. MaxiSorp plates (Nalge Nunc, Rochester N.Y.) were coated with 0.1 μg/ml human or mouse IL-12. A mixture of biotinylated ChDD2 (0.5 μg/ml final concentration) and competitor antibody (ChDD2 or HuDD2; starting at 0.5 or 1 mg/ml final concentration and serial 3-fold dilutions) in 100 μl ELISA buffer was added to an IL-12 coated well in triplicate. ELISA plates were incubated at room temperature for 2 hr. After washing the wells with Washing Buffer, 0.5 μg/ml HRP-conjugated streptavidin (Pierce) was added to each well. Color development was performed with TMB substrate (Kirkegaard & Perry Laboratories).

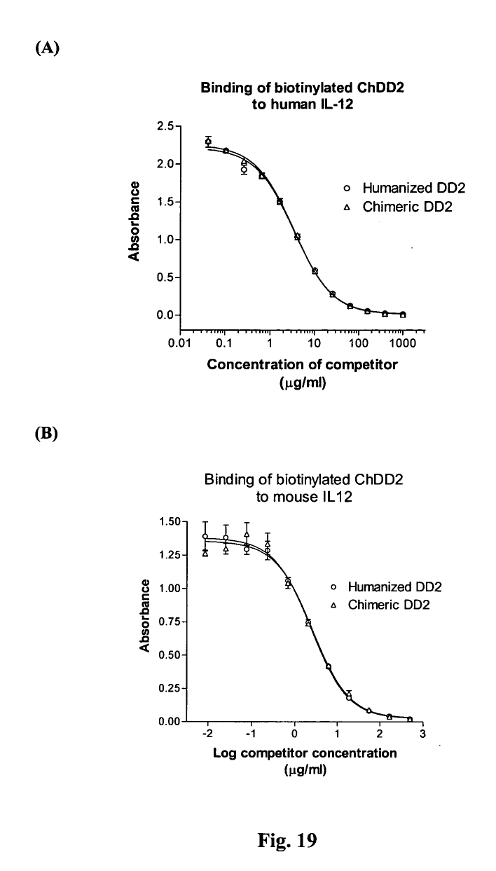

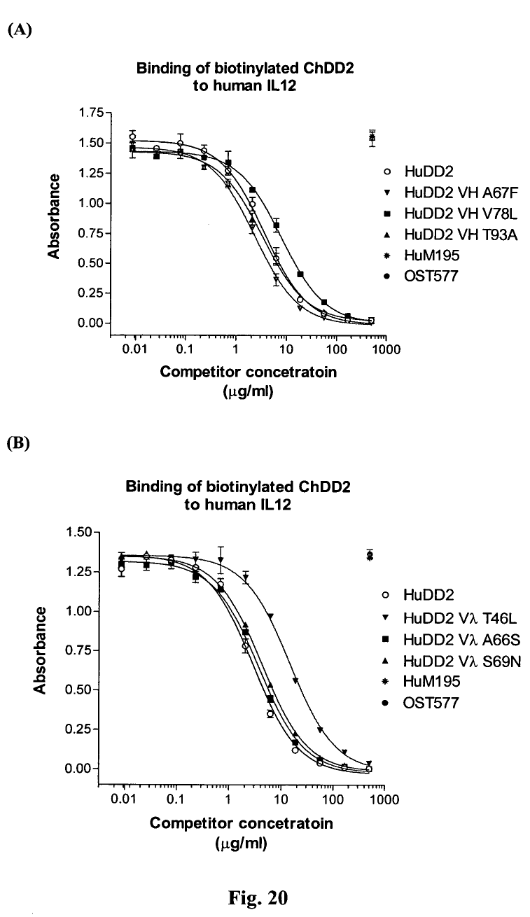

Representative results of the competition ELISA experiments are shown in FIG. 19. Both ChDD2 and HuDD2 competed with biotinylated ChDD2 for binding to human and mouse IL-12 in a concentration-dependent manner. The IC₅₀ values of ChDD2 and HuDD2 in binding to human IL-12, obtained using the computer software Prism (GraphPad Software Inc., San Diego, Calif.), were 4.0 μg/ml and 4.2 μg/ml, respectively. The IC₅₀ values for binding to mouse IL-12 were 2.4 μg/ml for both HuDD2 and ChDD2. These results clearly indicate that the affinity to human and mouse IL-12 was retained during the process of humanization of the chicken monoclonal antibody DD2.

The comparison of the amino acid sequences of chicken and human immunoglobulins revealed several important heavy and light chain variable region framework positions for the humanization design. While these framework amino acids are predicted to interact with the CDRs, they are often different between a chicken and a human. These positions include H67, H78, H93, L46, L66, and L69 (Kabat numbering). To assess the importance of these positions for humanization of chicken monoclonal antibodies, each of the chicken-derived amino acids at positions H67, H78, H93, L46, L66, and L69 in HuDD2 was replaced with the corresponding amino acid of the human acceptor framework. The HuDD2 VH mutants were designated A67F (replacement of Ala with Phe at position 67; see FIG. 15), V79L (replacement of Val with Leu at position 79), and T93A (replacement of Thr with Ala at position 93). The VH mutants were cloned into pVgl.d and cotransfected into Sp2/0 with pVλ2-HuDD2. The HuDD2λ mutants made were designated T46L (replacement of Thr with Leu at position 46), A66S (replacement of Ala with Ser at position 66), and S69N (replacement of Ser with Asn at position 69). These Vλ mutants were subcloned in to pVλ2 and cotransfected into Sp2/0 with pVg1-HuDD2.

The Sp2/0 stable transfectants expressing mutant HuDD2 were isolated as described in Example 3. Mutant HuDD2 antibodies were purified with protein A column chromatography as described in Example 3. The affinity of mutant HuDD2 to human IL-12 was analyzed by competition ELISA. A mixture of biotinylated ChDD2 (0.5 μg/ml final concentration) and competitor antibody (HuDD2 wild type or one of the mutants; starting at 0.5 mg/ml final concentration and serial 3-fold dilutions) in 100 μl ELISA buffer was added to ELISA plates coated with human IL-12 in triplicate. ELISA plates were incubated at room temperature for 2 hr. After washing the wells with Washing Buffer, 0.5 μg/ml HRP-conjugated streptavidin (Pierce) was added to each well. Color development was performed with TMB substrate (Kirkegaard & Perry Laboratories).

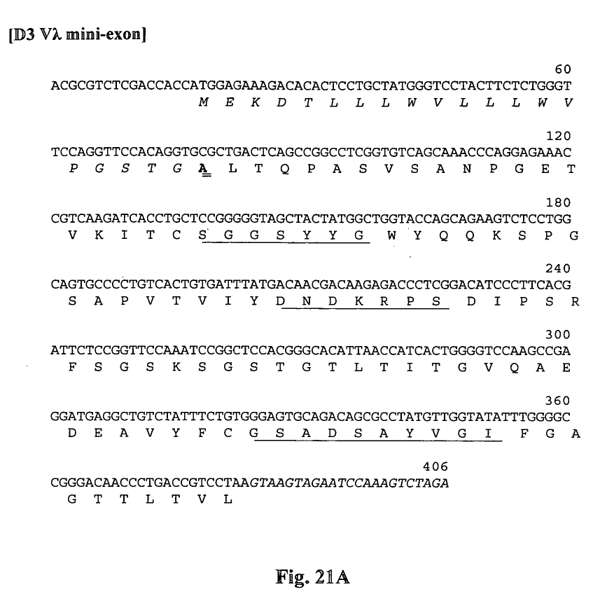

Representative results of the competition ELISA experiments are shown in FIG. 20. The IC50 values were 3.5 μg/ml for wild-type HuDD2, 2.1 μg/ml for HuDD2 VH A67F mutant, 7.0 μg/ml for HuDD2 VH V78L mutant, 3.3 μg/ml for HuDD2 VH T93A mutant, 16.1 μg/ml for HuDD2 Vλ T46L mutant, 3.4 μg/ml for HuDD2 Vλ A66S mutant, and 4.3 μg/ml for HuDD2 Vλ S69N mutant. The replacement of a chicken framework amino acid (Thr) with a human framework amino acid (Leu) at position 46 in the Vλ reduced the binding affinity to human IL-12 by 4.6 fold. In addition, the replacement of a chicken framework amino acid (Val) with a human framework amino acid (Leu) at position 78 in the VH reduced the binding affinity to human IL-12 by 2.0 fold. Therefore, these two locations (position 46 in Vλ and position 78 in VH) were found to be particularly important to retain the affinity of the chicken anti-IL12 monoclonal antibody DD2 in the humanized form.

### Example 5

This example describes the humanization of chicken anti-L-selectin antibodies.

Recombinant Soluble Human L-selectin, E-selectin, and P-selectin

The cDNA fragment encoding the extracellular region of human L-selectin (amino acids 1 through 279 of the mature protein) was obtained by PCR as described in Berg et al. (Blood 85:31-37 (1995)). The fragment was cloned downstream of the CMV promoter in a mammalian expression vector derived from pOKT3.Vk.rg (Cole, M. S., et al., J. Immunol. 159:3613-3621 (1997)) to make pDL117. After linearization, pDL117 plasmid DNA was introduced into the chromosome of the mouse myeloma cell line NS0 (European Collection of Animal Cell Cultures, Salisbury, Wiltshire, UK) by electroporation and NS0 stable transfectants were selected for gpt expression in mycophenolic acid media as described by Bebbington et al. (Biotechnology 10:169-175 (1992)). An NS0 stable transfectant producing a high level of soluble human L-selectin was adapted to and expanded in a serum-free medium using Hybridoma-SFM (Invitrogen). Soluble human L-selectin was purified by affinity column chromatography using agarose coupled with humanized anti-L-selectin monoclonal antibody HuDREG200 (Co et al., Immunotechnol. 4:253-266 (1999)).

The cDNA fragment encoding the extracellular region of human E-selectin (amino acids 1 through 282 of the mature protein) was obtained by PCR as described in Berg et al. (Blood 85:31-37 (1995)). The fragment was cloned downstream of the CMV promoter in a mammalian expression vector derived from pOKT3.Vk.rg (Cole, M. S., et al., J. Immunol. 159:3613-3621 (1997)) to make pDL173. After linearization, pDL173 plasmid DNA was introduced into the chromosome of the mouse myeloma cell line NS0 (European Collection of Animal Cell Cultures, Salisbury, Wiltshire, UK) by electroporation and NS0 stable transfectants were selected for gpt expression in mycophenolic acid media as described by Bebbington et al. (Biotechnology 10:169-175 (1992)). An NS0 stable transfectant producing a high level of truncated soluble human E-selectin was adapted to and expanded in a serum-free medium using Hybridoma-SFM (Invitrogen). Soluble human E-selectin was purified by affinity column chromatography using agarose coupled with humanized anti-E-/P-selectin monoclonal antibody HuEP5C7 (He et al., J. Immunol. 160:1693-1701 (1998)).

The cDNA fragment encoding the extracellular region of human P-selectin (amino acids 1 through 282 of the mature protein) was obtained by PCR as described in Berg et al. (Blood 85:31-37 (1995)). The fragment was cloned downstream of the CMV promoter in a mammalian expression vector derived from pOKT3.Vk.rg (Cole, M. S., et al., J. Immunol. 159:3613-3621 (1997)) to make pDL174. After linearization, pDL174 plasmid DNA was introduced into the chromosome of the mouse myeloma cell line NS0 European Collection of Animal Cell Cultures, Salisbury, Wiltshire, UK) by electroporation and NS0 stable transfectants were selected for gpt expression in mycophenolic acid media as described by Bebbington et al. (Biotechnology 10: 169-175 (1992)). An NS0 stable transfectant producing a high level of truncated soluble human P-selectin was adapted to and expanded in a serum-free medium using Hybridoma-SFM (Invitrogen). Soluble human P-selectin was purified by affinity column chromatography using agarose coupled with humanized anti-E-/P-selectin monoclonal antibody HuEP5C7 (He et al., J. Immunol. 160:1693-1701 (1998)).

Chicken Immunization

A female White Leghorn chicken was immunized with 200 μg of soluble human L-selectin in complete Freund adjuvant. The chicken was further immunized with 125 μg of soluble human P-selectin at day 7 in incomplete Freund adjuvant (IFA), 125 μg of soluble human E-selectin at day 14 in IFA, 50 μg of soluble L-selectin at day 21 in IFA, 50 μg of soluble P-selectin at day 28 in IFA, 50 pg of soluble E-selectin at day 35 in IFA, 50 μg of soluble L-selectin at day 42 in IFA, 50 μg of soluble P-selectin at day 49 in IFA, and 50 μg of soluble E-selectin at day 56 in IFA. The spleen was harvested on day 63. The chicken immunization was performed by BAbCO (Richmond, Calif.).

Isolation of Chicken Anti-L-selectin Monoclonal Antibodies

Construction of a chicken scFv library with the phage display vector pNT3206 was carried out as described in Example 1. Isolation of phage antibodies that bind to human L-selectin was carried out by panning as described (Akamatsu, Y., et al., J. Immunol. 151: 4651-4659 (1993)). After three cycles of binding to and elution from soluble human L-selectin coated in an ELISA plate, infection of TG1 ΔrecA, and rescue by superinfection of VCSM13 helper phage, several phage clones were found to produce scFv antibodies that bind specifically to human L-selectin. One of the clones, D3, bound strongly to human L-selectin, but not to human E-selectin or P-selectin.

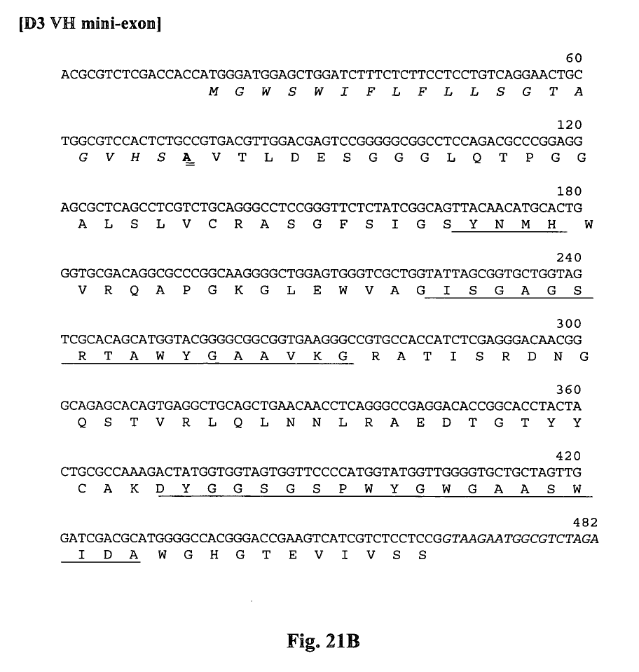

To express D3 in the form of whole antibody, each of the Vλ and VH genes in the phage display vector was converted to an exon structure containing a signal peptide and a splicing donor site as described in Example 2 (also outlined in FIG. 6). After digestion with MluI and XbaI, the Vλ and VH genes were cloned into the corresponding sites of the mammalian expression vectors pVλ1 (described in Example 1) and pVg1.d (Co, M. S., et al., J. Immunol. 148: 1149-1154 (1992)), respectively. The nucleotide and deduced amino acid sequences of chicken D3 Vλ and VH mini exons are shown in FIG. 21. The resultant vectors were named pVλ1-D3 for light chain expression and pVg1.d-D3 for heavy chain expression.

Design of Humanized D3 Variable Regions

For humanization of chicken D3 variable regions, the general approach provided in the present invention was followed. First, a molecular model of the D3 variable regions was constructed with the aid of the computer programs ABMOD (Zilber, B., Scherf, T., Levitt, M., and Anglister, J. Biochemistry 29:10032-41 (1990)) and ENCAD (Levitt, M., J. Mol. Biol. 168: 595-620 (1983)). Next, based on a homology search against human V region sequences, the rearranged Vλ gene 3-23OIIIB237 (Ignatovich, et al., Genbank accession no. Z85114) and the J segment Jλ2 (Udey, J. A. and Blomberg, B., Immunogenetics 25: 63-70 (1987)) were selected to provide the frameworks for the D3 light chain variable region. For the D3 heavy chain variable region, the rearranged VH gene ha316 (Lai, et al., J. Autoimmunity 11:39-51 (1998)) was used. The identity of the framework amino acids between chicken D3 Vλ region and the acceptor human Vλ gene 3-23OIIIB237 -Jλ2 segment was 69%, while the identity between chicken D3 VH and the human VH gene ha316 was 69%.

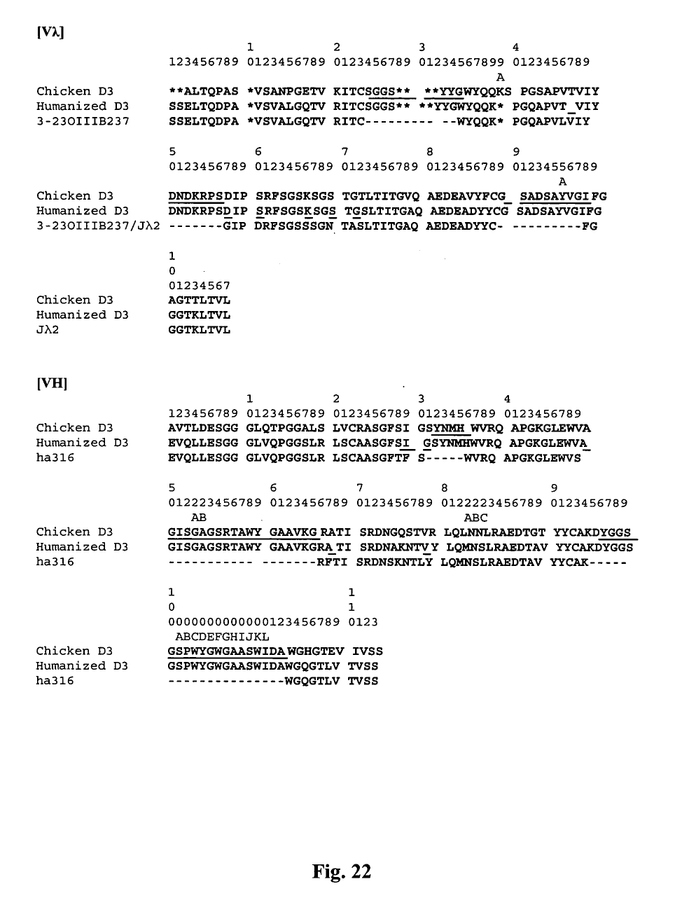

Chicken Vλ regions contain two amino acid deletions and one amino acid insertion compared to human Vλ sequences (FIG. 22). The N-terminal amino acid of chicken mature Vλ corresponds to the third amino acid from the N-terminus of human Vλ. The framework 2 of chicken Vλ contains an extra amino acid at position 39A. Although the first two amino acids at the N-terminus of human mature Vλ exists in the close proximity of the CDR, detailed examination of the model suggests that the transfer of the two serine residues from the human Vλ gene 3-23OIIIB237 to the N-terminus of D3 Vλ would not drastically change the CDR structure. A serine residue at position 39A in the chicken D3 Vλ is located in the loop opposite and away from the antigen binding site. The model predicted that the removal of a serine at position 39A during humanization would not introduce a significant change in the CDR or framework structure. Therefore, in the humanized D3 Vλ sequence, the two N-terminal amino acids of the human Vλ gene 3-23OIIIB237 were added and a serine residue at position 39A in the chicken D3 Vλ was eliminated.

At framework positions in which the computer model suggested significant contact with the CDRs, the amino acids from the chicken V regions were substituted for the original human framework amino acids. This was performed at residues 46, 57, 60, 66, 69 and 71 of the light chain (FIG. 22). For the heavy chain, replacements were made at residues 28, 29, 30, 49, 67 and 78 (FIG. 22). In addition, a serine residue at position 74 in the humanized D3 VH was substituted with an alanine residue, in order to achieve better antibody expression in mammalian cells (Co, et al., Cancer Res. 56:1118-25 (1996)). The alignment of the amino acid sequences of the chicken D3, humanized D3 and human acceptor framework sequences are shown segments for both light and heavy variable regions in FIG. 22.

Expression of Humanized D3 IgG1/λ Antibody

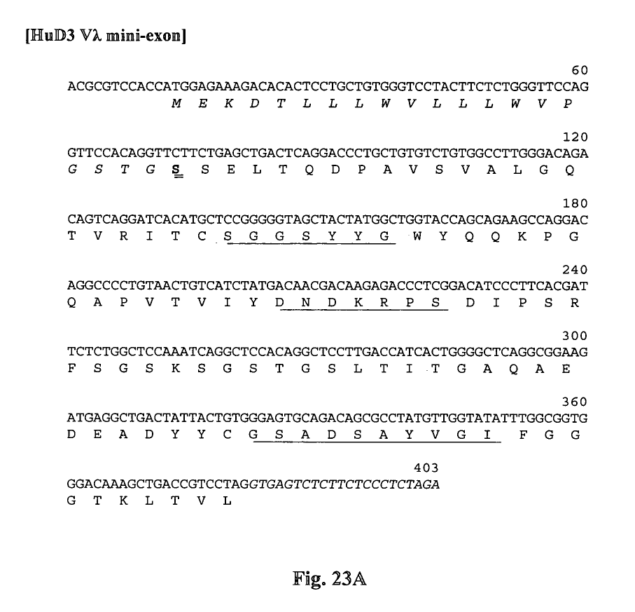

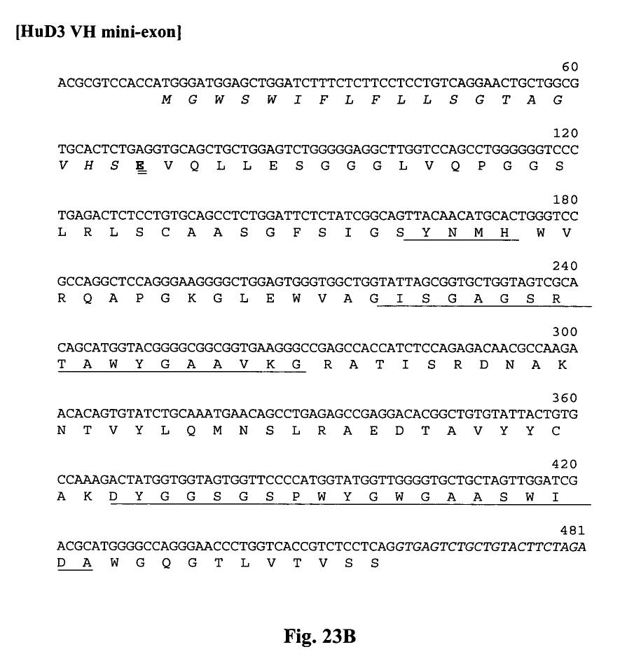

The humanized D3 Vλ and VH genes, designed as a mini-exon including a signal peptide, a splice donor signal, and appropriate restriction enzyme sites, were synthesized at GenScript Corporation (Edison, N.J.). The V gene fragments were then digested with MluI and XbaI, and subcloned into mammalian expression vectors, pVλ1 and pVg1.d (described in Example 1), respectively. The resultant plasmids were designated pVλ1-HuD3 and pVg1.d-HuD3. The nucleotide sequence and deduced amino acid sequence of the light (A) or heavy (B) chain variable region of humanized anti-L-selectin antibody D3 are shown in FIG. 23.

Humanized anti-L-selectin IgGI/k antibody D3 (HuD3) was transiently expressed by cotransfection of pVλ1-HuD3 and pVg1.d-HuD3 into 293-H cells (Invitrogen) using the Lipofectamine 2000 reagent (Invitrogen). Similarly, chicken-human chimeric anti-L-selectin IgG1/k antibody D3 (ChD3) was transiently expressed in 293-H cells by cotransfection of pVλ1-D3 and pVg1.d-D3 using Lipofectamin 2000 (Invitrogen). Transiently transfected 293-H cells were grown in DME medium containing 10% FBS. The expression levels of ChD3 and HuD3 in the culture supernatants of transiently transfected 293-H cells were measured by sandwich ELISA as described in Example 3.

Binding Properties of Humanized D3

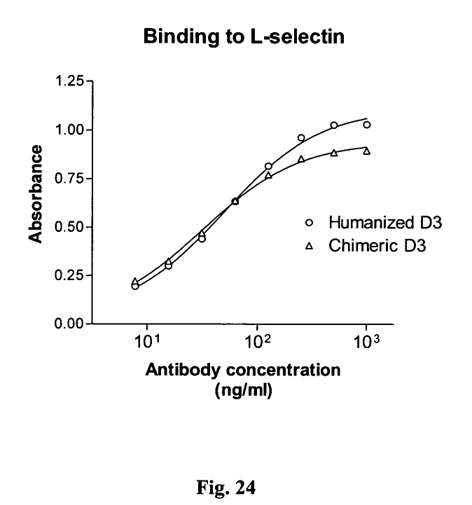

Binding of ChD3 and HuD3 in culture supernatants of transiently transfected 293-H cells to human L-selectin was examined by ELISA. MaxiSorp plates (Nalge Nunc, Rochester N.Y.) were coated with 0.25 μg/ml of soluble human L-selectin in 0.2 M sodium carbonate buffer (pH 9.4) and blocked with SuperBlock buffer (Pierce). Various concentrations of ChD3 or HuD3 in ELISA buffer (PBS containing 1% BSA and 0.1% Tween 20), starting at 0.5 mg/ml final concentration and serial 2.5-fold dilutions, were added to L-selectin-coated ELISA plates in triplicate. ChD3 and HuD3 bound to L-selectin were detected by incubation with HRP-conjugated goat anti-human λ chain antibodies (SouthemBiotech, Birmingham, Ala.). Color development was performed with TMB substrate. As shown in FIG. 24, both ChD3 and HuD3 bound well to human L-selectin, indicating that the affinity of chicken-human chimeric antibody D3 to human L-selectin was retained in the humanized form.

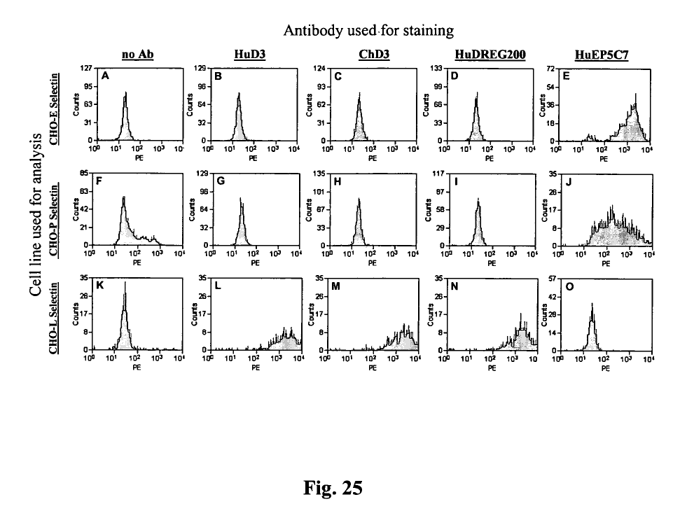

The specificity of ChD3 and HuD3 in binding to human L-selectin was examined by flow cytometry using CHO-K1 transfectant cell lines expressing human E-selectin (CHO-E Selectin), P-selectin (CHO-P selectin), or L-selectin (CHO-L selectin) on the surface (Berg et al., Blood 85:31-37 (1995)). 2×10⁵ cells were incubated on ice for 30 minutes in FACS buffer (PBS containing 1% bovine serum albumin and 0.2% sodium azide) with 1 μg of test antibody or buffer alone. After incubation, cells were washed three times with FACS buffer and subsequently incubated for another 30 minutes with PE-conjugated donkey anti-human IgG F(ab′)2 fragment (Jackson ImmunoResearch). After washing three times with FACS buffer, relative cell fluorescence was analyzed by flow cytometry using a Cyan (DAKOCytomation). As shown in FIG. 25, a control antibody HuDREG200, a humanized anti-human L-selectin IgG1/κ monoclonal antibody (Co et al., Immunotechol. 4:253-266 (1999)), showed binding to CHO-L selectin (panel N), but not to CHO-E selectin (panel D) or CHO-P selectin (panel I). Another control antibody HuEP5C7, a humanized anti-E-/P-selectin IgG1/κ monoclonal antibody (ref), showed binding to CHO-E selectin (panel E) and CHO-P selectin (panel J), but not to CHO-L selectin. ChD3 bound to CHO-L-selectin (panel M), but not to CHO-E selectin (panel C) or CHO-E selectin (panel H). HuD3 also bound to CHO-L-selectin (panel L), but not to CHO-E selectin (panel B) or CHO-P selectin (panel G). This result indicates that the antigen specificity of ChD3 was not lost after conversion to the humanized form.

The affinity of ChD3 and HuD3 to human L-selectin was further characterized by competition ELISA. 293-H cells were transiently transfected with the expression vectors for ChD3 or HuD3 as described above and grown in DME medium containing 5% low Ig FBS (HyClone). ChD3 and HuD3 in exhausted culture supemantats were purified by protein A column chromatography as described in Example 3. ELISA plates were coated with 0.25 μg/ml soluble human L-selectin in 0.2 M sodium carbonate buffer (pH 9.4) and blocked with SuperBlock blocking buffer (Pierce). A mixture of biotinylated ChD3 (0.5 μg/ml final concentration) and competitor antibody (ChD3 or HuD3 starting at 1 mg/ml final concentration and serial 2.5-fold dilutions) in 100 μl ELISA buffer were added in triplicate. As controls, humanized anti-human L-selectin monoclonal antibody HuDREG55 (Co et al., Immunotechol. 4:253-266 (1999)) and humanized anti-human HLA-DR monoclonal antibody Hu1D10 (Kostelny et al., Int. J. Cancer 93:556-565 (2001)) were used. ELISA plates were incubated at room temperature for 2 hr. After washing the wells with Washing Buffer (PBS containing 0.1% Tween 20), 100 μl of 1 μg/ml HRP-conjugated streptavidin (Pierce) in ELISA buffer was added to each well. ELISA plates were incubated at room temperature for 60 min and washed with Washing Buffer. For color development, 100 μl/well of ABTS substrate was added. Color development was stopped by adding 100 μl/well of 2% oxalic acid. Absorbance was read at 415 nm.

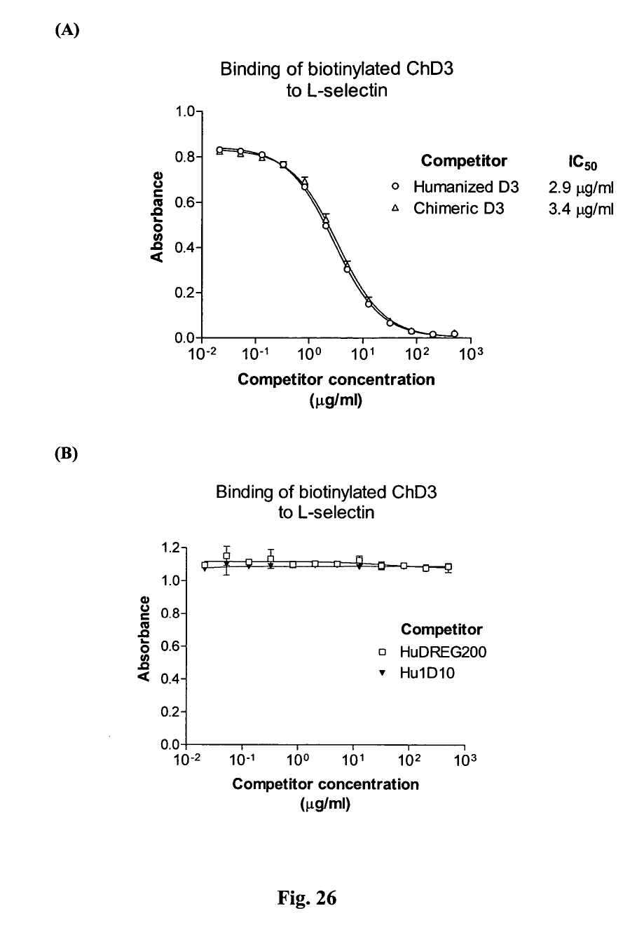

Two control antibodies, HuDREG55 and Hu1D10, did not compete with ChD3 in binding to human L-selectin (FIG. 26B). On the other hand, both ChD3 and HuD3 competed with biotinylated ChD3 in a concentration-dependent manner (FIG. 26A). The IC₅₀ values of HuD3 and ChD3, obtained using the computer software Prism (GraphPad Software Inc., San Diego, Calif.), were 2.9 μg/ml and 3.4 μg/ml, respectively. These results indicate that humanization of ChD3 was successful; the antigen affinity and specificity of the chicken anti-L-selectin monoclonal antibody D3 was retained in the humanized form.

### Example 6

This example describes humanization of chicken monoclonal antibodies that bind to and/or neutralize more than one member of the selectin family proteins.

Immunization of a chicken with human L-selectin, E-selectin, and P-selectin is performed as described in Example 5. A phage display library for chicken anti-selectin scFv antibodies is constructed as described in Examples 1 and 5. Isolation of phage antibodies that bind to more than one member of the selectin family proteins is performed by panning as described in Examples 1 and 5. For example, to isolate the clones that bind to L-selectin and P-selectin, L-selectin is used as antigen at the first and third cycles of panning and P-selectin as antigen at the second and fourth cycles of panning. To isolate the clones that bind to E-selectin and L-selectin, E-selectin is used as antigen at the first and third cycles of panning and L-selectin at the second and fourth cycles of panning. To isolate the clones that bind to E-selectin, P-selectin and L-selectin, E-selectin is used as antigen at the first and fourth rounds of panning, P-selectin at the second and fifth rounds, and L-selectin at the third and sixth rounds.

Isolated phage antibodies that bind to more than one member of the selectin family proteins are converted to the whole antibody form as described in Example 2. Chicken-human chimeric IgG/λ antibodies are expressed transiently in 293-H cells or stable in Sp2/0 cells as described in Examples 3 and 5. Chicken-human chimeric antibodies are characterized in binding affinity, antigen specificity, and neutralization activity, for example, as described in Co et al. (Immunotechol. 4:253-266 (1999)), He et al. (J. Immunol., 160:1029-1035 (1998)), and Berg et al. (Blood, 85, 31-37 (1995)).

Humanization of chicken antibodies that bind to and/or neutralize more than one member of the selectin family proteins is carried out according to the general guideline described in this work. Humanized chicken antibodies are tested in binding affinity by competition ELISA as described in Example 5.

Although the invention has been described with reference to the presently preferred embodiments, it is understood that various modifications may be made without departing from the spirit of the invention.

All publications, patents, patent applications, and web sites are herein incorporated by reference in their entirety to the same extent as if each individual patent, patent application, or web site was specifically and individually indicated to be incorporated by reference in its entirety.

1. A humanized immunoglobulin having complementarity determining regions (CDRs) from a donor immunuglobulin, heavy chain variable region frameworks from a human acceptor immunoglobulin, and light chain variable region frameworks from a human acceptor immunoglobulin, wherein said humanized immunoglobulin specifically binds to an antigen of the donor immunoglobulin, wherein said donor immunoglobulin is a chicken immunoglobulin.

2. The humanized immunoglobulin according to claim 1, wherein said humanized immunoglobulin comprises amino acids from the donor immunoglobulin framework outside the CDRs of the humanized immunoglobulin that replace the corresponding amino acids in the acceptor immunoglobulin heavy or light chain frameworks, and each of these said donor amino acids is capable of interacting with the CDRs.

3. The humanized immunoglobulin according to claim 1, wherein an amino acid of the human acceptor immunuglobulin framework is replaced by a human immunoglobulin consensus amino acid at its position, wherein the replaced amino acid is rare for human immunoglobulin sequences at its position.

4. The humanized immunoglobulin according to claim 1, wherein a human acceptor immunuglobulin framework residue in at least one position selected from the group consisting of H67, H78, H93, L46, L66, and L69 is replaced.

5. The humanized immunoglobulin according to claim 4, wherein said position or positions is occupied by an amino acid in an equivalent position of the chicken donor immunoglobulin.

6. The humanized immunoglobulin according to claim 1, wherein amino acid sequence of the acceptor immunoglobulin heavy chain variable framework is at least 60% identical to that of the donor immunoglobulin.

7. The humanized immunoglobulin according to claim 1, wherein said humanized immunoglobulin is capable of binding to a first antigen derived from human as well as a second antigen derived from a non-human mammal, wherein the second antigen is substantially identical to the first antigen.

8. The humanized immunoglobulin according to claim 7, wherein said non-human mammal is a mouse.

9. The humanized immunoglobulin according to claim 1, wherein said humanized immunoglobulin specifically binds to the antigen of the donor immunoglobulin with an affinity constant of at least 10⁸ M⁻¹.

10. The humanized immunoglobulin according to claim 1, wherein said humanized immunoglobulin binds to or neutralizes both human and mouse IL-12 or L-selectin.

11. The humanized immunoglobulin according to claim 10, wherein at least one position selected from the group consisting of H47, H68, H79, L44, L55, L58, L64, and L67 of the humanized immunoglobulin is occupied by an amino acid in an equivalent position of the chicken donor immunoglobulin.

12. The humanized immunoglobulin according to claim 10, wherein at least one position selected from the group consisting of H78, L7, L9, L70 and L76 of the humanized immunoglobulin is occupied by a consensus amino acid in the human acceptor immunoglobulin.

13. The humanized immunoglobulin according to claim 12, wherein H78 is occupied by threonine, L7 is occupied by proline, L9 is occupied by serine, L70 is occupied by threonine, and L76 is occupied by valine.

14. The humanized immunoglobulin according to claim 10,
wherein positions H1-H30 of the heavy chain framework of the humanized immunoglobulin have an amino acid sequence comprising at least 85% sequence identity to SEQ ID NO: 5; positions H36-H49 of the heavy chain framework have an amino acid sequence comprising at least 85% sequence identity to SEQ ID NO: 6; positions H66-H94 of the heavy chain framework have an amino acid sequence comprising at least 85% sequence identity to SEQ ID NO: 7; and positions H103-H113 of the heavy chain framework have an amino acid sequence comprising at least 85% sequence identity to SEQ ID NO: 8; and
wherein positions L1-L22 of the light chain framework have an amino acid sequence comprising at least 85% sequence identity to SEQ ID NO: 9; position L35-L49 of the light chain framework have an amino acid sequence comprising at least 85% sequence identity to SEQ ID NO: 10; positions L57-L88 of the light chain framework have an amino acid sequence comprising at least 85% sequence identity to SEQ ID NO: 11; and positions L98-L107 of the light chain framework have an amino acid sequence comprising at least 85% sequence identity to SEQ ID NO: 12.

15. The humanized immunoglobulin according to claim 14, wherein said humanized immunoglobulin binds to or neutralizes human and mouse IL-12, wherein said donor immunoglobulin is a chicken donor immunoglobulin having a heavy chain variable region as presented in SEQ ID NO: 2 or 48 and a light chain variable region as presented in SEQ ID NO: 4 or 47.

16. The humanized immunoglobulin according to claim 14, wherein said humanized immunoglobulin binds to or neutralizes human and mouse L-selectin, wherein said donor immunoglobulin is a chicken donor immunoglobulin having a heavy chain variable region as presented in SEQ ID NO: 82 and a light chain variable region as presented in SEQ ID NO: 80.

17. A chicken antibody that binds to or neutralizes human and mouse IL-12 or L-selectin.

18. The chicken antibody according to claim 17, comprising a heavy chain variable region as presented in SEQ ID NO: 2 or 48 and a light chain variable region as presented in SEQ ID NO: 4 or 47.

19. The chicken antibody according to claim 17, comprising a heavy chain variable region as presented in SEQ ID NO: 82 and a light chain variable region as presented in SEQ ID NO: 80.

20. A chimeric antibody capable of binding to human and mouse IL-12, wherein said chimeric antibody comprises a variable region derived from a chicken antibody and a constant region derived from a human antibody.

21. The chimeric chicken antibody according to claim 20, comprising:
(a) a heavy chain variable region as presented in SEQ ID NO:2 or 48;
(b) a light chain variable region as presented in SEQ ID NO:4 or 47; and
(c) a heavy chain and a light chain constant region of a human IgG1.

22. A chimeric antibody capable of binding to human and mouse L-selectin, wherein said chimeric antibody comprises a variable region is derived from a chicken antibody and a constant region derived from a human antibody.

23. The chimeric chicken antibody according to claim 22, comprising:
(a) a heavy chain variable region as presented in SEQ ID NO: 82;
(b) a light chain variable region as presented in SEQ ID NO: 80; and
(c) a heavy chain and a light chain constant region of a human IgG1.

24. A polypeptide comprising SEQ ID NO: 2, SEQ ID NO: 4, SEQ ID NO: 14, SEQ ID NO: 16, SEQ ID NO: 47, SEQ ID NO: 48, SEQ ID NO: 49, SEQ ID NO: 50, SEQ ID NO: 80, SEQ ID NO:82.

25. A polynucleotide molecule comprising SEQ ID NO: 1, SEQ ID NO: 3, SEQ ID NO: 13, SEQ ID NO: 15, SEQ ID NO: 71, SEQ ID NO: 73, SEQ ID NO: 83, or SEQ ID NO: 85.

26. An expression vector comprising the polynucleotide molecule according to claim 25.

27. A host cell comprising the expression vector according to claim 26.

28. A pharmaceutical composition comprising the humanized immunoglobulin according to claim 9 and a pharmaceutical carrier.

29. A method of treating an autoimmune disease in a subject in need of such a treatment comprising administering to said subject a therapeutically effective amount of the pharmaceutical composition according to claim 28.

30. A method of producing a humanized chicken immunoglobulin comprising:
(a) preparing expression vectors comprising DNA segments encoding a heavy chain variable region of the humanized chicken immunoglobulin having complementarity determining regions (CDRs) from a donor chicken immunoglobulin and heavy chain variable region frameworks from a human acceptor immunoglobulin, and/or DNA segments encoding a light chain variable region of the humanized chicken immunoglobulin having complementarity determining regions (CDRs) from the donor chicken immunoglobulin and light chain variable region and frameworks from the human acceptor immunoglobulin;
(b) transforming host cells with said vector(s); and
(c) culturing said transformed host cells to produce said humanized chicken immunoglobulin.

31. The method according to claim 30, wherein said humanized chicken immunoglobulin comprises amino acids from the donor chicken immunoglobulin framework outside the CDRs of the humanized immunoglobulin that replace the corresponding amino acids in the acceptor immunoglobulin heavy or light chain frameworks, and each of these said donor amino acids is capable of interacting with the CDRs.

32. The method according to claim 30, wherein the amino acid of the human acceptor immunuglobulin framework is replaced by a human immunoglobulin consensus amino acid at its position, wherein the replaced amino acid is rare for human immunoglobulin sequences at its position.

33. The method according to claim 30, wherein a residue in at least one position selected from the group consisting of H67, H78, H93, L46, L66, and L69 of the human acceptor immunuglobulin framework is replaced.


SEQUENCE LISTING (103 sequences)

SEQ ID NO: 1 - DNA, 378 bp, Chicken
tgccgtgacgttggacgagtctgggggcggcctccagacgcccggaggagcgctcagcctcgtctgcaaggcctccgggttcaccttcagtagttacagcatgctctgggtgcgacaggcgcccggcaaggggctggaatacgtcgctgaaattaccaacactggtaggaccagaagatacggggcggcggtgaagggccgtgccaccatctcgagggacaacgggcagagcacagtgaggctgcagctgaacaacctcagggctgaggacaccggcacctactactgcgccagaagtagtgtttattcttgttcttatggttggtgtgctggtaacatcaacgcatggggccacgggaccgaagtcatcgtctcctc

SEQ ID NO: 2 - protein, 126 aa, Chicken
AVTLDESGGGLQTPGGALSLVCKASGFTFSSYSMLWVRQAPGKGLEYVAEITNTGRTRRYGAAVKGRATISRDNGQSTVRLQLNNLRAEDTGTYYCARSSVYSCSYGWCAGNINAWGHGTEVIVSS

SEQ ID NO: 3 - DNA, 312 bp, chicken
tgcgctgactcagccggcctcagtgtcagcaaacctgggaggaaccgtcaagatcacctgctccgggggttacagcggctattatggctggtaccagcagaaatcacctggcagtgcccctgtcactgtgatctatgacaacaccaggagaccctcggacatcccttcacgattctccggttccaaatccggctccacagccacattaaccatcactggggtccaagccgacgacgaggctgtctatttctgtgggacctgggacagcagccgtgttggtatatttggggccgggacaaccctgaccgtcct

SEQ ID NO: 4 - protein, 104 aa, Chicken
ALTQPASVSANLGGTVKITCSGGYSGYYGWYQQKSPGSAPVTVIYDNTRRPSDIPSRFSGSKSGSTATLTITGVQADDEAVYFCGTWDSSRVGIFGAGTTLTVL

SEQ ID NO: 5 - protein, 30 aa, Homo sapiens
EVQLVESGGGLVQPGGSLRLSCAASGFTFS

SEQ ID NO: 6 - protein, 14 aa, Homo sapiens
WVRQAPGKGLEWVA

SEQ ID NO: 7 - protein, 32 aa, Homo sapiens
RFTISRDNAKNSLYLQMNSLRAEDTAVYYCAR

SEQ ID NO: 8 - protein, 9 aa, Homo sapiens
WGQGTLVTV

SEQ ID NO: 9 - protein, 22 aa, Homo sapiens
SSELTQDPAVSVALGQTVRITC

SEQ ID NO: 10 - protein, 15 aa, Homo sapiens
WYQQKPGQAPVLVIY

SEQ ID NO: 11 - protein, 32 aa, Homo sapiens
GIPDRFSGSSSGNTASLTITGAQAEDEADYYC

SEQ ID NO: 12 - protein, 11 aa, Homo sapiens
FGGGTKLTVLG

SEQ ID NO: 13 - DNA, 378 bp, Homo sapiens
tgaggtgcagttggtggagtccggaggtggactcgtgcagcctggaggaagcctcaggctcagctgcgccgcctccgggttcaccttcagtagttacagcatgctctgggtgcgacaggcgcctggcaagggactggaatacgtcgctgaaattaccaacactggtaggaccagaagatacggagctgcggtgaagggccgtgccaccatctcgagggacaacgccaagaacacagtgtacctgcagatgaacagcctcagggctgaggacaccgccgtgtactactgcgccagaagtagtgtttattcttgttcttatggttggtgtgctggtaacatcaacgcatggggccagggaaccctggtcaccgtctcctc

SEQ ID NO: 14 - protein, 126 aa, Homo sapiens
EVQLVESGGGLVQPGGSLRLSCAASGFTFSSYSMLWVRQAPGKGLEYVAEITNTGRTRRYGAAVKGRATISRDNAKNTVYLQMNSLRAEDTAVYYCARSSVYSCSYGWCAGNINAWGQGTLVTVSS

SEQ ID NO: 15 - DNA, 315 bp, Homo sapiens
tagctctgagctgactcagccgccttcagtgtcagtggccctgggacagaccgtcaggatcacctgctccggaggttacagcggctattatggctggtaccagcagaaacctggccaggctcctgtcactgtgatttatgacaacaccaggagaccctcggacatcccttcacgattctccggttccaaatccggctccacagccacattaaccatcactggagtccaagccgaggacgaggctgactattactgtgggacctgggacagcagccgtgttggtatatttggaggtgggacaaagctgaccgtcct

SEQ ID NO: 16 - protein, 105 aa, Homo sapiens
SSELTQPPSVSVALGQTVRITCSGGYSGYYGWYQQKPGQAPVTVIYDNTRRPSDIPSRFSGSKSGSTATLTITGVQAEDEADYYCGTWDSSRVGIFGGGTKLTVL

SEQ ID NO: 17 - DNA, 27 bp, Artificial
  Synthetic Primer
gggggcgccagcggctcgccctgtgtc

SEQ ID NO: 18 - DNA, 31 bp, Artificial
  Synthetic Primer
cccggcgccgacaacggtttggagggacctc

SEQ ID NO: 19 - DNA, 30 bp, Artificial
  Synthetic Primer
gggtctagagccattggactctccgtcctg

SEQ ID NO: 20 - DNA, 33 bp, Artificial
  Synthetic Primer
cccgctcagccctccaaattttcatcctggatc

SEQ ID NO: 21 - DNA, 45 bp, Artificial
  Synthetic Primer
cttcgtgctagcgtccactccaatatagatgtgtgcaagcttggc

SEQ ID NO: 22 - DNA, 33 bp, Artificial
  Synthetic Primer
ctgagccacaccggtgttggctttgccctgtgg

SEQ ID NO: 23 - DNA, 37 bp, Artificial
  Synthetic Primer
ccagcacccatggccgccgtgacgttggacgagtccg

SEQ ID NO: 24 - DNA, 36 bp, Artificial
  Synthetic Primer
cgtcaagctagcggaggagacgatgacttcggtccc

SEQ ID NO: 25 - DNA, 37 bp, Artificial
  Synthetic Primer
cacgcagagctcgcgctgactcagccgtgcctcgagt

SEQ ID NO: 26 - DNA, 34 bp, Artificial
  Synthetic Primer
agccacagatcttaggacggtcagggttgtcccg

SEQ ID NO: 27 - DNA, 70 bp, Artificial
  Synthetic Primer
ctagccacgcgtccaccatggagaaagacacactcctgctatgggtcctacttctctgggttccaggttc

SEQ ID NO: 28 - DNA, 70 bp, Artificial
  Synthetic Primer
ccagggccactgacactgaaggcggctgagtcagctcagagctacctgtggaacctggaacccagagaag

SEQ ID NO: 29 - DNA, 71 bp, Artificial
  Synthetic Primer
cttcagtgtcagtggccctgggacagaccgtcaggatcacctgctccggaggttacagcggctattatggc

SEQ ID NO: 30 - DNA, 71 bp, Artificial
  Synthetic Primer
gttgtcataaatcacagtgacaggagcctggccaggtttctgctggtaccagccataatagccgctgtaac

SEQ ID NO: 31 - DNA, 73 bp, Artificial
  Synthetic Primer
cctgtcactgtgatttatgacaacaccaggagaccctcggacatcccttcacgattctccggttccaaatccg

SEQ ID NO: 32 - DNA, 69 bp, Artificial
  Synthetic Primer
cctcgtcctcggcttggactccagtgatggttaatgtggctgtggagccggatttggaaccggagaatc

SEQ ID NO: 33 - DNA, 73 bp, Artificial
  Synthetic Primer
gagtccaagccgaggacgaggctgactattactgtgggacctgggacagcagccgtgttggtatatttggagg

SEQ ID NO: 34 - DNA, 73 bp, Artificial
  Synthetic Primer
gactcgtctagagggagaagagactcacctaggacggtcagctttgtcccacctccaaatataccaacacggc

SEQ ID NO: 35 - DNA, 20 bp, Artificial
  Synthetic Primer
ctagccacgcgtccaccatg

SEQ ID NO: 36 - DNA, 20 bp, Artificial
  Synthetic Primer
gactcgtctagagggagaag

SEQ ID NO: 37 - DNA, 78 bp, Artificial
  Synthetic Primer
ctagccacgcgtccaccatgggatggagctggatctttctcttcctcctgtcaggaactgctggcgtccactctcagg

SEQ ID NO: 38 - DNA, 77 bp, Artificial
  Synthetic Primer
gagcctgaggcttcctccaggctgcacgagtccacctccggactccaccaactgcacctgagagtggacgccagcag

SEQ ID NO: 39 - DNA, 79 bp, Artificial
  Synthetic Primer
cctggaggaagcctcaggctcagctgcgccgcctccgggttcaccttcagtagttacagcatgctctgggtgcgacagg

SEQ ID NO: 40 - DNA, 78 bp, Artificial
  Synthetic Primer
cttctggtcctaccagtgttggtaatttcagcgacgtattccagtcccttgccaggcgcctgtcgcacccagagcatg

SEQ ID NO: 41 - DNA, 79 bp, Artificial
  Synthetic Primer
ccaacactggtaggaccagaagatacggagctgcggtgaagggccgtgccaccatctctagggacaacgccaagaacac

SEQ ID NO: 42 - DNA, 79 bp, Artificial
  Synthetic Primer
ggcgcagtagtacacggcggtgtcctcagccctgaggctgttcatctgcaggtacactgtgttcttggcgttgtcccta

SEQ ID NO: 43 - DNA, 77 bp, Artificial
  Synthetic Primer
ccgccgtgtactactgcgccagaagtagtgtttattcttgttcttatggttggtgtgctggtaacatcaacgcatgg

SEQ ID NO: 44 - DNA, 79 bp, Artificial
  Synthetic Primer
gactcgtctagaggttgtgaggactcaccggaggagacggtgaccagggttccctggccccatgcgttgatgttaccag

SEQ ID NO: 45 - DNA, 20 bp, Artificial
  Synthetic Primer
ctagccacgcgtccaccatg

SEQ ID NO: 46 - DNA, 21 bp, Artificial
  Synthetic Primer
gactcgtctagaggttgtgag

SEQ ID NO: 47 - protein, 108 aa, Chicken
ALTQPASVSANPGETVKITCPGGGIYAGRYYGYGWFQQKSPGSAPVTVIYSNDKRPSDIPSRFSGSASGSTATLTITGVQADDEAVYFCGSHDSNVGVFGAGTTLTVL

SEQ ID NO: 48 - protein, 136 aa, Chicken
AVTLDESGGGLQTPGGGLSLVCKASGFDFSNYQLQWVRQAPGKGLEWVGGIGSSGSSTYYGAAVKGRATISRDNGQSTVRLQLNNLRAEDTGTYYCTRGVSPYSCWYAGRTSYTCHAYTAGSIDAWGHGTEVIVSS

SEQ ID NO: 49 - protein, 109 aa, Chicken
SSELTQDPAVSVALGQTVRITCPGGGIYAGRYYGYGWFQQKPGQAPVTVIYSNDKRPSDIPSRFSGSASGSTASLTITGAQAEDEADYYCGSHDSNVGVFGGGTKLTVL

SEQ ID NO: 50 - protein, 136 aa, Chicken
EVQLVESGGGLVQPGGSLRLSCAASGFDFSNYQLQWVRQAPGKGLEWVGGIGSSGSSTYYGAAVKGRATISRDNAKNSVYLQMNSLRAEDTAVYYCTRGVSPYSCWYAGRTSYTCHAYTAGSIDAWGQGTLVTVSS

SEQ ID NO: 51 - DNA, 73 bp, Artificial
  Synthetic Primer
acgcgtccaccatggagaaagacacactcctgctgtgggtcctacttctctgggttccaggttccacaggttc

SEQ ID NO: 52 - DNA, 75 bp, Artificial
  Synthetic Primer
cctgactgtctgtcccaaggccacagacacagcagggtcctgagtcagctcagaagaacctgtggaacctggaac

SEQ ID NO: 53 - DNA, 73 bp, Artificial
  Synthetic Primer
ccttgggacagacagtcaggatcacatgccccggaggtggcatctatgctggacgctactatggttatggctg

SEQ ID NO: 54 - DNA, 70 bp, Artificial
  Synthetic Primer
cgttgctatagatgacagttacaggggcctgtcctggcttctgctggaaccagccataaccatagtagcg

SEQ ID NO: 55 - DNA, 70 bp, Artificial
  Synthetic Primer
ctgtaactgtcatctatagcaacgacaagagaccctcggacatcccttcacgattctctggctccgcatc

SEQ ID NO: 56 - DNA, 69 bp, Artificial
  Synthetic Primer
catcttccgcctgagccccagtgatggtcaaggaagctgtggagcctgatgcggagccagagaatcgtg

SEQ ID NO: 57 - DNA, 66 bp, Artificial
  Synthetic Primer
ggctcaggcggaagatgaggctgactattactgtgggagccacgacagcaatgttggtgtatttgg

SEQ ID NO: 58 - DNA, 71 bp, Artificial
  Synthetic Primer
tctagagggagaagagactcacctaggacggtcagctttgtcccaccgccaaatacaccaacattgctgtc

SEQ ID NO: 59 - DNA, 27 bp, Artificial
  Synthetic Primer
ctacgaacgcgtccaccatggagaaag

SEQ ID NO: 60 - DNA, 29 bp, Artificial
  Synthetic Primer
gacttctctagagggagaagagactcacc

SEQ ID NO: 61 - DNA, 80 bp, Artificial
  Synthetic Primer
acgcgtccaccatgggatggagctggatctttctcttcctcctgtcaggaactgctggcgtgcactctgaggtgcagctg

SEQ ID NO: 62 - DNA, 80 bp, Artificial
  Synthetic Primer
ggctgcacaggagagtctcagggaccccccaggctggaccaagcctcccccagactccaccagctgcacctcagagtgca

SEQ ID NO: 63 - DNA, 80 bp, Artificial
  Synthetic Primer
tgagactctcctgtgcagcctctggattcgactttagtaactatcagttgcagtgggtccgccaggctccagggaagggg

SEQ ID NO: 64 - DNA, 80 bp, Artificial
  Synthetic Primer
aaccgcagctccgtagtatgtgctactgccactgctgccaataccacccacccactccagccccttccctggagcctggc

SEQ ID NO: 65 - DNA, 80 bp, Artificial
  Synthetic Primer
catactacggagctgcggttaagggccgagccaccatctccagagacaacgccaagaactcagtgtatctgcaaatgaac

SEQ ID NO: 66 - DNA, 80 bp, Artificial
  Synthetic Primer
ctgtaaggactaacacctctggtacagtaatacacagccgtgtcctcggctctcaggctgttcatttgcagatacactga

SEQ ID NO: 67 - DNA, 80 bp, Artificial
  Synthetic Primer
agaggtgttagtccttacagctgttggtatgccggccgtactagttatacttgtcatgcatatactgctggtagcatcga

SEQ ID NO: 68 - DNA, 80 bp, Artificial
  Synthetic Primer
tctagaagtacagcagactcacctgaggagacggtgaccagggttccctggccccatgcgtcgatgctaccagcagtata

SEQ ID NO: 69 - DNA, 26 bp, Artificial
  Synthetic Primer
ctacgaacgcgtccaccatgggatgg

SEQ ID NO: 70 - DNA, 28 bp, Artificial
  Synthetic Primer
gacttctctagaagtacagcagactcac

SEQ ID NO: 71 - DNA, 421 bp, Chicken
acgcgtccaccatggagaaagacacactcctgctgtgggtcctacttctctgggttccaggttccacaggttcttctgagctgactcaggaccctgctgtgtctgtggccttgggacagacagtcaggatcacatgccccggaggtggcatctatgctggacgctactatggttatggctggttccagcagaagccaggacaggcccctgtaactgtcatctatagcaacgacaagagaccctcggacatcccttcacgattctctggctccgcatcaggctccacagcttccttgaccatcactggggctcaggcggaagatgaggctgactattactgtgggagccacgacagcaatgttggtgtatttggcggtgggacaaagctgaccgtcctaggtgagtctcttctccctctaga

SEQ ID NO: 72 - protein, 129 aa, Chicken
MEKDTLLLWVLLLWVPGSTGSSELTQDPAVSVALGQTVRITCPGGGIYAGRYYGYGWFQQKPGQAPVTVIYSNDKRPSDIPSRFSGSASGSTASLTITGAQAEDEADYYCGSHDSNVGVFGGGTKLTVL

SEQ ID NO: 73 - DNA, 499 bp, Chicken
acgcgtccaccatgggatggagctggatctttctcttcctcctgtcaggaactgctggcgtgcactctgaggtgcagctggtggagtctgggggaggcttggtccagcctggggggtccctgagactctcctgtgcagcctctggattcgactttagtaactatcagttgcagtgggtccgccaggctccagggaaggggctggagtgggtgggtggtattggcagcagtggcagtagcacatactacggagctgcggttaagggccgagccaccatctccagagacaacgccaagaactcagtgtatctgcaaatgaacagcctgagagccgaggacacggctgtgtattactgtaccagaggtgttagtccttacagctgttggtatgccggccgtactagttatacttgtcatgcatatactgctggtagcatcgacgcatggggccagggaaccctggtcaccgtctcctcaggtgagtctgctgtacttctaga

SEQ ID NO: 74 - protein, 155 aa, Chicken
MGWSWIFLFLLSGTAGVHSEVQLVESGGGLVQPGGSLRLSCAASGFDFSNYQLQWVRQAPGKGLEWVGGIGSSGSSTYYGAAVKGRATISRDNAKNSVYLQMNSLRAEDTAVYYCTRGVSPYSCWYAGRTSYTCHAYTAGSIDAWGQGTLVTVSS

SEQ ID NO: 75 - DNA, 406 bp, Chicken
acgcgtctcgaccaccatggagaaagacacactcctgctatgggtcctacttctctgggttccaggttccacaggtgcgctgactcagccggcctcggtgtcagcaaacccaggagaaaccgtcaagatcacctgctccgggggtagctactatggctggtaccagcagaagtctcctggcagtgcccctgtcactgtgatttatgacaacgacaagagaccctcggacatcccttcacgattctccggttccaaatccggctccacgggcacattaaccatcactggggtccaagccgaggatgaggctgtctatttctgtgggagtgcagacagcgcctatgttggtatatttggggccgggacaaccctgaccgtcctaagtaagtagaatccaaagtctaga

SEQ ID NO: 76 - protein, 122 aa, Chicken
MEKDTLLLWVLLLWVPGSTGALTQPASVSANPGETVKITCSGGSYYGWYQQKSPGSAPVTVIYDNDKRPSDIPSRFSGSKSGSTGTLTITGVQAEDEAVYFCGSADSAYVGIFGAGTTLTVL

SEQ ID NO: 77 - DNA, 482 bp, Chicken
acgcgtctcgaccaccatgggatggagctggatctttctcttcctcctgtcaggaactgctggcgtccactctgccgtgacgttggacgagtccgggggcggcctccagacgcccggaggagcgctcagcctcgtctgcagggcctccgggttctctatcggcagttacaacatgcactgggtgcgacaggcgcccggcaaggggctggagtgggtcgctggtattagcggtgctggtagtcgcacagcatggtacggggcggcggtgaagggccgtgccaccatctcgagggacaacgggcagagcacagtgaggctgcagctgaacaacctcagggccgaggacaccggcacctactactgcgccaaagactatggtggtagtggttccccatggtatggttggggtgctgctagttggatcgacgcatggggccacgggaccgaagtcatcgtctcctccggtaagaatggcgtctaga

SEQ ID NO: 78 - protein, 149 aa, Chicken
MGWSWIFLFLLSGTAGVHSAVTLDESGGGLQTPGGALSLVCRASGFSIGSYNMHWVRQAPGKGLEWVAGISGAGSRTAWYGAAVKGRATISRDNGQSTVRLQLNNLRAEDTGTYYCAKDYGGSGSPWYGWGAASWIDAWGHGTEVIVSS

SEQ ID NO: 79 - protein, 102 aa, Chicken
ALTQPASVSANPGETVKITCSGGSYYGWYQQKSPGSAPVTVIYDNDKRPSDIPSRFSGSKSGSTGTLTITGVQAEDEAVYFCGSADSAYVGIFGAGTTLTVL

SEQ ID NO: 80 - protein, 103 aa, Homo sapiens
SSELTQDPAVSVALGQTVRITCSGGSYYGWYQQKPGQAPVTVIYDNDKRPSDIPSRFSGSKSGSTGSLTITGAQAEDEADYYCGSADSAYVGIFGGGTKLTVL

SEQ ID NO: 81 - protein, 130 aa, Chicken
AVTLDESGGGLQTPGGALSLVCRASGFSIGSYNMHWVRQAPGKGLEWVAGISGAGSRTAWYGAAVKGRATISRDNGQSTVRLQLNNLRAEDTGTYYCAKDYGGSGSPWYGWGAASWIDAWGHGTEVIVSS

SEQ ID NO: 82 - protein, 130 aa, Homo sapiens
EVQLLESGGGLVQPGGSLRLSCAASGFSIGSYNMHWVRQAPGKGLEWVAGISGAGSRTAWYGAAVKGRATISRDNAKNTVYLQMNSLRAEDTAVYYCAKDYGGSGSPWYGWGAASWIDAWGQGTLVTVSS

SEQ ID NO: 83 - DNA, 403 bp, Homo sapiens
acgcgtccaccatggagaaagacacactcctgctgtgggtcctacttctctgggttccaggttccacaggttcttctgagctgactcaggaccctgctgtgtctgtggccttgggacagacagtcaggatcacatgctccgggggtagctactatggctggtaccagcagaagccaggacaggcccctgtaactgtcatctatgacaacgacaagagaccctcggacatcccttcacgattctctggctccaaatcaggctccacaggctccttgaccatcactggggctcaggcggaagatgaggctgactattactgtgggagtgcagacagcgcctatgttggtatatttggcggtgggacaaagctgaccgtcctaggtgagtctcttctccctctaga

SEQ ID NO: 84 - protein, 123 aa, Homo sapiens
MEKDTLLLWVLLLWVPGSTGSSELTQDPAVSVALGQTVRITCSGGSYYGWYQQKPGQAPVTVIYDNDKRPSDIPSRFSGSKSGSTGSLTITGAQAEDEADYYCGSADSAYVGIFGGGTKLTVL

SEQ ID NO: 85 - DNA, 481 bp, Homo sapiens
acgcgtccaccatgggatggagctggatctttctcttcctcctgtcaggaactgctggcgtgcactctgaggtgcagctgctggagtctgggggaggcttggtccagcctggggggtccctgagactctcctgtgcagcctctggattctctatcggcagttacaacatgcactgggtccgccaggctccagggaaggggctggagtgggtggctggtattagcggtgctggtagtcgcacagcatggtacggggcggcggtgaagggccgagccaccatctccagagacaacgccaagaacacagtgtatctgcaaatgaacagcctgagagccgaggacacggctgtgtattactgtgccaaagactatggtggtagtggttccccatggtatggttggggtgctgctagttggatcgacgcatggggccagggaaccctggtcaccgtctcctcaggtgagtctgctgtacttctaga

SEQ ID NO: 86 - protein, 149 aa, Homo sapiens
MGWSWIFLFLLSGTAGVHSEVQLLESGGGLVQPGGSLRLSCAASGFSIGSYNMHWVRQAPGKGLEWVAGISGAGSRTAWYGAAVKGRATISRDNAKNTVYLQMNSLRAEDTAVYYCAKDYGGSGSPWYGWGAASWIDAWGQGTLVTVSS

SEQ ID NO: 87 - DNA, 6 bp, Artificial
  Restriction site
acgcgt

SEQ ID NO: 88 - DNA, 6 bp, Artificial
  Restriction site
tctaga

SEQ ID NO: 89 - protein, 79 aa, Homo sapiens
SSELTQDPAVSVALGQTVRITCWYQQKPGQAPVLVIYGIPDRFSGSSSGNTASLTITGAQAEDEADYYCFGGGTKLTVL

SEQ ID NO: 90 - protein, 87 aa, Homo sapiens
EVQLVESGGGLVQPGGSLRLSCAASGFTFSWVRQAPGKGLEWVARFTISRDNAKNSLYLQMNSLRAEDTAVYYCARWGQGTLVTVSS

SEQ ID NO: 91 - DNA, 412 bp, Chicken
acgcgtctcgaccaccatggagaaagacacactcctgctatgggtcctacttctctgggttccaggttccacaggtgcgctgactcagccggcctcagtgtcagcaaacctgggaggaaccgtcaagatcacctgctccgggggttacagcggctattatggctggtaccagcagaaatcacctggcagtgcccctgtcactgtgatctatgacaacaccaggagaccctcggacatcccttcacgattctccggttccaaatccggctccacagccacattaaccatcactggggtccaagccgacgacgaggctgtctatttctgtgggacctgggacagcagccgtgttggtatatttggggccgggacaaccctgaccgtcctaagtaagtagaatccaaagtctaga

SEQ ID NO: 92 - protein, 124 aa, Chicken
MEKDTLLLWVLLLWVPGSTGALTQPASVSANLGGTVKITCSGGYSGYYGWYQQKSPGSAPVTVIYDNTRRPSDIPSRFSGSKSGSTATLTITGVQADDEAVYFCGTWDSSRVGIFGAGTTLTVL

SEQ ID NO: 93 - DNA, 470 bp, Chicken
acgcgtctcgaccaccatgggatggagctggatctttctcttcctcctgtcaggaactgctggcgtccactctgccgtgacgttggacgagtctgggggcggcctccagacgcccggaggagcgctcagcctcgtctgcaaggcctccgggttcaccttcagtagttacagcatgctctgggtgcgacaggcgcccggcaaggggctggaatacgtcgctgaaattaccaacactggtaggaccagaagatacggggcggcggtgaagggccgtgccaccatctcgagggacaacgggcagagcacagtgaggctgcagctgaacaacctcagggctgaggacaccggcacctactactgcgccagaagtagtgtttattcttgttcttatggttggtgtgctggtaacatcaacgcatggggccacgggaccgaagtcatcgtctcctccggtaagaatggcgtctaga

SEQ ID NO: 94 - protein, 145 aa, Chicken
MGWSWIFLFLLSGTAGVHSAVTLDESGGGLQTPGGALSLVCKASGFTFSSYSMLWVRQAPGKGLEYVAEITNTGRTRRYGAAVKGRATISRDNGQSTVRLQLNNLRAEDTGTYYCARSSVYSCSYGWCAGNINAWGHGTEVIVSS

SEQ ID NO: 95 - DNA, 409 bp, Homo sapiens
acgcgtccaccatggagaaagacacactcctgctatgggtcctacttctctgggttccaggttccacaggtagctctgagctgactcagccgccttcagtgtcagtggccctgggacagaccgtcaggatcacctgctccggaggttacagcggctattatggctggtaccagcagaaacctggccaggctcctgtcactgtgatttatgacaacaccaggagaccctcggacatcccttcacgattctccggttccaaatccggctccacagccacattaaccatcactggagtccaagccgaggacgaggctgactattactgtgggacctgggacagcagccgtgttggtatatttggaggtgggacaaagctgaccgtcctaggtgagtctcttctccctctaga

SEQ ID NO: 96 - protein, 125 aa, Homo sapiens
MEKDTLLLWVLLLWVPGSTGSSELTQPPSVSVALGQTVRITCSGGYSGYYGWYQQKPGQAPVTVIYDNTRRPSDIPSRFSGSKSGSTATLTITGVQAEDEADYYCGTWDSSRVGIFGGGTKLTVL

SEQ ID NO: 97 - DNA, 469 bp, Homo sapiens
acgcgtccaccatgggatggagctggatctttctcttcctcctgtcaggaactgctggcgtccactctgaggtgcagttggtggagtccggaggtggactcgtgcagcctggaggaagcctcaggctcagctgcgccgcctccgggttcaccttcagtagttacagcatgctctgggtgcgacaggcgcctggcaagggactggaatacgtcgctgaaattaccaacactggtaggaccagaagatacggagctgcggtgaagggccgtgccaccatctcgagggacaacgccaagaacacagtgtacctgcagatgaacagcctcagggctgaggacaccgccgtgtactactgcgccagaagtagtgtttattcttgttcttatggttggtgtgctggtaacatcaacgcatggggccagggaaccctggtcaccgtctcctccggtgagtcctcacaacctctaga

SEQ ID NO: 98 - protein, 145 aa, Homo sapiens
MGWSWIFLFLLSGTAGVHSEVQLVESGGGLVQPGGSLRLSCAASGFTFSSYSMLWVRQAPGKGLEYVAEITNTGRTRRYGAAVKGRATISRDNAKNTVYLQMNSLRAEDTAVYYCARSSVYSCSYGWCAGNINAWGQGTLVTVSS

SEQ ID NO: 99 - DNA, 423 bp, Chicken
acgcgtctcgaccaccatggagaaagacacactcctgctatgggtcctacttctctgggttccaggttccacaggtgcgctgactcagccggcctcagtgtcagcaaacccgggagaaaccgtcaagatcacctgccccgggggtggcatctatgctggaaggtactatggttatggctggttccagcagaagtctcctggcagtgcccctgtcactgtgatctatagcaacgacaagagaccctcggacatcccttcacgattctccggctccgcatccggctccacagccacattaaccatcactggggtccaagccgacgacgaggctgtctatttctgtgggagccacgacagcaatgttggtgtatttggggccgggacaaccctgaccgtcctaagtaagtagaatccaaatctaga

SEQ ID NO: 100 - protein, 128 aa, Chicken
MEKDTLLLWVLLLWVPGSTGALTQPASVSANPGETVKITCPGGGIYAGRYYGYGWFQQKSPGSAPVTVIYSNDKRPSDIPSRFSGSASGSTATLTITGVQADDEAVYFCGSHDSNVGVFGAGTTLTVL

SEQ ID NO: 101 - DNA, 500 bp, Chicken
acgcgtctcgaccaccatgggatggagctggatctttctcttcctcctgtcaggaactgctggcgtccactctgccgtgacgttggacgagtccgggggcggcctccagacgcccggaggagggctcagcctcgtctgcaaggcctccgggttcgacttcagcaactatcagttgcagtgggtgcgccaggcgcccggcaaggggctggagtgggtcggtggtattggcagcagtggcagtagcacatactacggggcggcggtgaagggccgtgccaccatctcgagggacaacgggcagagcacagtgagactgcagctgaacaacctcagggctgaggacaccggcacctactactgcaccagaggtgttagtccttacagctgttggtatgccggccgtactagttatacttgtcatgcatatactgctggtagcatcgacgcatggggccacgggaccgaagtcatcgtctcctccggtaagaatggcgtctaga

SEQ ID NO: 102 - protein, 155 aa, Chicken
MGWSWIFLFLLSGTAGVHSAVTLDESGGGLQTPGGGLSLVCKASGFDFSNYQLQWVRQAPGKGLEWVGGIGSSGSSTYYGAAVKGRATISRDNGQSTVRLQLNNLRAEDTGTYYCTRGVSPYSCWYAGRTSYTCHAYTAGSIDAWGHGTEVIVSS

SEQ ID NO: 103 - protein, 87 aa, Homo sapiens
EVQLLESGGGLVQPGGSLRLSCAASGFTFSWVRQAPGKGLEWVSRFTISRDNSKNTLYLQMNSLRAEDTAVYYCAKWGQGTLVTVSS